SEQUENCE LISTING (3 sequences)

SEQ ID NO: 1 - DNA, 13 bp, Artificial Sequence
  Synthetic Construct
gcaagcatcctgt

SEQ ID NO: 2 - DNA, 16 bp, Artificial Sequence
  Synthetic Construct
gagttacttgccaact

SEQ ID NO: 3 - DNA, 13 bp, Artificial Sequence
  Synthetic Construct
gcattggtattca

(19) United States
(12) Patent Application Publication
(10) Pub. No.: US 2022/0073916 A1
(43) Pub. Date: Mar. 10, 2022

(54) NOVEL PHOSPHORAMIDITES

(71) Applicant: Roche Innovation Center Copenhagen A/S, Horsholm (DK)

(72) Inventors: Konrad BLEICHER, Freiburg (DE); Jessica Marine Aurore BASTIEN, Basel (CH)

(21) Appl. No.: 17/405,708

(22) Filed: Aug. 18, 2021

Related U.S. Application Data
(63) Continuation of application No. PCT/EP2020/054410, filed on Feb. 20, 2020.

(30) Foreign Application Priority Data
Feb. 20, 2019 (EP) 19158303.8

(51) Int. Cl. C12N 15/113; C07H 21/02; C07H 19/067

(52) U.S. Cl. CPC C12N 15/113; C07H 21/02; C12N 2320/30; C12N 2310/11; C12N 2310/341; C07H 19/067

(57) ABSTRACT

The invention relates to a compound of formula (II)

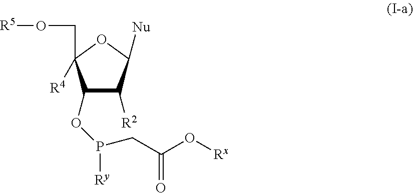

wherein X, Y, R⁵, R^{x}, R^{y} and Nu are as defined in the description and in the claims. The compound of formula (II) can be used in the manufacture of medicaments.

### SEQUENCE LISTING

The instant application contains a Sequence Listing which has been submitted electronically in ASCII format and is hereby incorporated by reference in its entirety. Said ASCII copy, created on Aug. 3, 2021, is named 51551-013001_Sequence_Listing_8_3_21_ST25 and is 832 bytes in size.

The invention relates in particular to a single-stranded antisense gapmer oligonucleotide comprising at least one dinucleoside of formula (I)

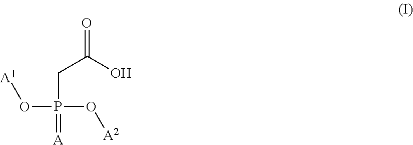

wherein one of (A¹) and (A²) is a sugar-modified nucleoside and the other one is a sugar-modified nucleoside or a DNA nucleoside and A is oxygen or sulfur, or a pharmaceutically acceptable salt thereof.

The invention relates also in particular to novel phosphoramidites useful in preparing the antisense gapmer oligonucleotide according to the invention.

Synthetic oligonucleotides as therapeutic agents have witnessed remarkable progress over recent years leading to a broad portfolio of clinically validated molecules acting by diverse mechanisms including RNase H activating gapmers, splice switching oligonucleotides, microRNA inhibitors, siRNA or aptamers (S. T. Crooke, Antisense drug technology: principles, strategies, and applications, 2nd ed. ed., Boca Raton, Fla.: CRC Press, 2008). Natural oligonucleotides are inherently unstable towards nucleolytic degradation in biological systems. Furthermore, they show a highly unfavorable pharmacokinetic behavior. In order to improve on these drawbacks a wide variety of chemical modifications have been investigated in recent decades. Arguably one of the most successful modifications is the introduction of phosphorothioate linkages, where one of the non-bridging phosphate oxygen atoms is replaced with a sulfur atom (F. Eckstein, Antisense and Nucleic Acid Drug Development 2009, 10, 117-121). Such phosphorothioate oligodeoxynucleotides show an increased protein binding as well as a distinctly higher stability to nucleolytic degradation and thus a substantially higher half-life in plasma, tissues and cells than their unmodified phosphodiester analogues. These crucial features have allowed for the development of the first generation of oligonucleotide therapeutics as well as paved the way for their further improvement through later generation modifications such as Locked Nucleic Acids (LNAs).

It was surprisingly found that the single-stranded antisense oligonucleotide according to the invention was well-tolerated. They were at least as potent in vitro as the reference oligonucleotide comprising phosphorothioate internucleoside linkages only and more potent in vivo than the reference oligonucleotide comprising phosphorothioate internucleoside linkages only. Surprisingly also, the single-stranded antisense oligonucleotide according to the invention was particularly potent in heart cell lines (in vitro) and hear tissue (in vivo).

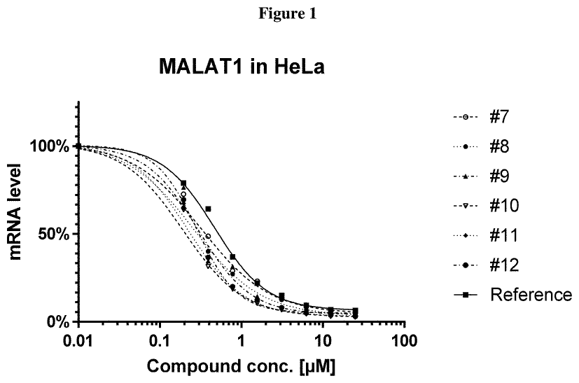

FIG. 1 shows a dose-response curve of oligonucleotides according to the invention targeting MALAT1 mRNA in human HeLa cell lines

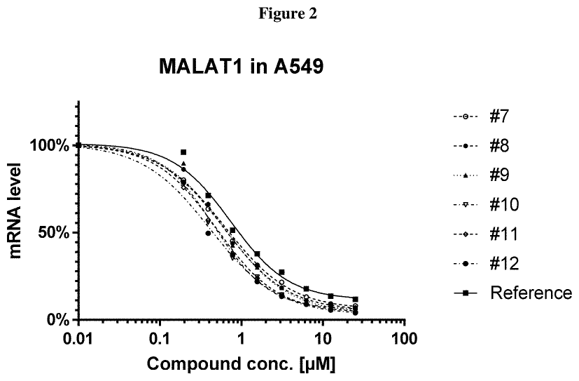

FIG. 2 shows a dose-response curve of oligonucleotides according to the invention targeting MALAT1 mRNA in human A549 cell lines.

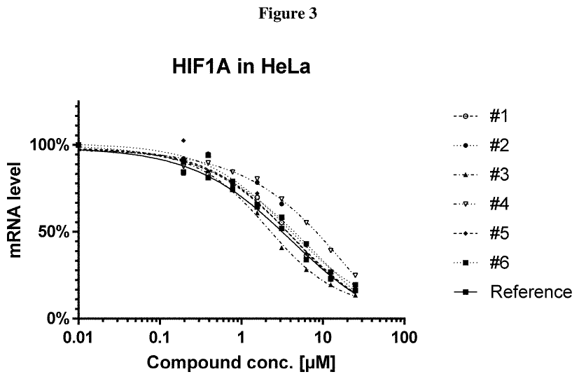

FIG. 3 shows a dose-response curve of oligonucleotides according to the invention targeting HIF1A mRNA in human HeLa cell lines.

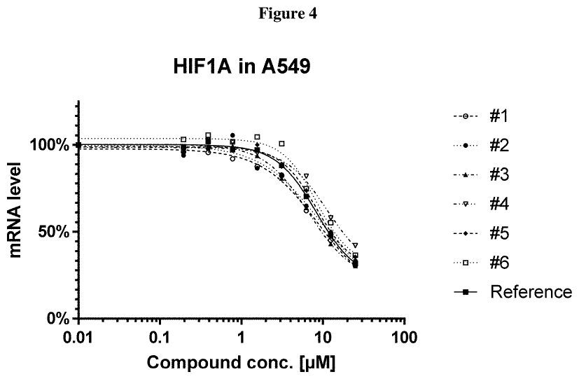

FIG. 4 shows a dose-response curve of oligonucleotides according to the invention targeting HIF1A mRNA in human A549 cell lines.

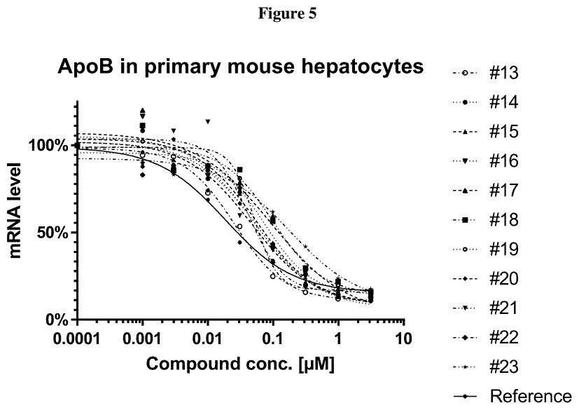

FIG. 5 shows a dose-response curve of oligonucleotides according to the invention targeting ApoB mRNA in mouse primary hepatocytes.

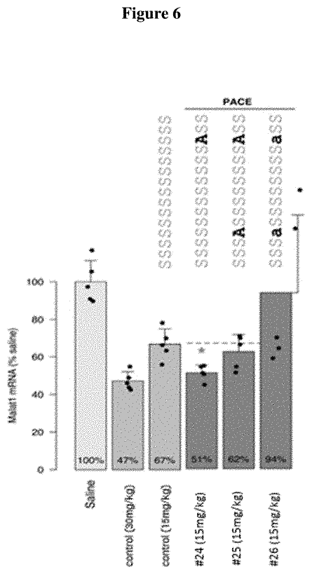

FIG. 6 shows the amount of Malat1 mRNA levels in heart of animals treated with an oligonucleotide according to the invention.

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C₁-C₈ alkyl groups are methyl, ethyl, propyl, isopropyl, butyl, isobutyl, tert-butyl, the isomeric pentyls, the isomeric hexyls, the isomeric heptyls and the isomeric octyls, particularly methyl, ethyl, propyl, butyl and pentyl. Particular examples of alkyl are methyl, ethyl and propyl.

The term “cycloalkyl”, alone or in combination, signifies a cycloalkyl ring with 3 to 8 carbon atoms and particularly a cycloalkyl ring with 3 to 6 carbon atoms. Examples of cycloalkyl are cyclopropyl, cyclobutyl, cyclopentyl, cyclohexyl, cycloheptyl and cyclooctyl, more particularly cyclopropyl and cyclobutyl. A particular example of “cycloalkyl” is cyclopropyl.

The term “alkoxy”, alone or in combination, signifies a group of the formula alkyl-O— in which the term “alkyl” has the previously given significance, such as methoxy, ethoxy, n-propoxy, isopropoxy, n-butoxy, isobutoxy, sec.butoxy and tert.butoxy. Particular “alkoxy” are methoxy and ethoxy. Methoxyethoxy is a particular example of “alkoxy alkoxy”.

The term “oxy”, alone or in combination, signifies the —O— group.

The term “alkenyl”, alone or in combination, signifies a straight-chain or branched hydrocarbon residue comprising an olefinic bond and up to 8, preferably up to 6, particularly preferred up to 4 carbon atoms. Examples of alkenyl groups are ethenyl, 1-propenyl, 2-propenyl, isopropenyl, 1-butenyl, 2-butenyl, 3-butenyl and isobutenyl.

The term “alkynyl”, alone or in combination, signifies a straight-chain or branched hydrocarbon residue comprising a triple bond and up to 8, particularly 2 carbon atoms.

The terms “halogen” or “halo”, alone or in combination, signifies fluorine, chlorine, bromine or iodine and particularly fluorine, chlorine or bromine, more particularly fluorine. The term “halo”, in combination with another group, denotes the substitution of said group with at least one halogen, particularly substituted with one to five halogens, particularly one to four halogens, i.e. one, two, three or four halogens.

The term “haloalkyl”, alone or in combination, denotes an alkyl group substituted with at least one halogen, particularly substituted with one to five halogens, particularly one to three halogens. Examples of haloalkyl include monofluoro-, difluoro- or trifluoromethyl, -ethyl or -propyl, for example 3,3,3-trifluoropropyl, 2-fluoroethyl, 2,2,2-trifluoroethyl, fluoromethyl or trifluoromethyl. Fluoromethyl, difluoromethyl and trifluoromethyl are particular “haloalkyl”.

The term “halocycloalkyl”, alone or in combination, denotes a cycloalkyl group as defined above substituted with at least one halogen, particularly substituted with one to five halogens, particularly one to three halogens. Particular example of “halocycloalkyl” are halocyclopropyl, in particular fluorocyclopropyl, difluorocyclopropyl and trifluorocyclopropyl.

The terms “hydroxyl” and “hydroxy”, alone or in combination, signify the —OH group.

The terms “thiohydroxyl” and “thiohydroxy”, alone or in combination, signify the —SH group.

The term “carbonyl”, alone or in combination, signifies the —C(O)— group.

The term “carboxy” or “carboxyl”, alone or in combination, signifies the —COOH group.

The term “amino”, alone or in combination, signifies the primary amino group (—NH₂), the secondary amino group (—NH—), or the tertiary amino group (—N—).

The term “alkylamino”, alone or in combination, signifies an amino group as defined above substituted with one or two alkyl groups as defined above.

The term “sulfonyl”, alone or in combination, means the —SO₂ group.

The term “sulfinyl”, alone or in combination, signifies the —SO— group.

The term “sulfanyl”, alone or in combination, signifies the —S— group.

The term “cyano”, alone or in combination, signifies the —CN group.

The term “azido”, alone or in combination, signifies the —N₃ group.

The term “nitro”, alone or in combination, signifies the NO₂ group.

The term “formyl”, alone or in combination, signifies the —C(O)H group.

The term “carbamoyl”, alone or in combination, signifies the —C(O)NH₂ group.

The term “cabamido”, alone or in combination, signifies the —NH—C(O)—NH₂ group.

The term “aryl”, alone or in combination, denotes a monovalent aromatic carbocyclic mono- or bicyclic ring system comprising 6 to 10 carbon ring atoms, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples of aryl include phenyl and naphthyl, in particular phenyl.

The term “heteroaryl”, alone or in combination, denotes a monovalent aromatic heterocyclic mono- or bicyclic ring system of 5 to 12 ring atoms, comprising 1, 2, 3 or 4 heteroatoms selected from N, O and S, the remaining ring atoms being carbon, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples of heteroaryl include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, azepinyl, diazepinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, quinoxalinyl, carbazolyl or acridinyl.

The term “heterocyclyl”, alone or in combination, signifies a monovalent saturated or partly unsaturated mono- or bicyclic ring system of 4 to 12, in particular 4 to 9 ring atoms, comprising 1, 2, 3 or 4 ring heteroatoms selected from N, O and S, the remaining ring atoms being carbon, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples for monocyclic saturated heterocyclyl are azetidinyl, pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Examples for bicyclic saturated heterocycloalkyl are 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, or 3-thia-9-aza-bicyclo[3.3.1]nonyl. Examples for partly unsaturated heterocycloalkyl are dihydrofuryl, imidazolinyl, dihydro-oxazolyl, tetrahydro-pyridinyl or dihydropyranyl.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, poly amine resins. The oligonucleotide of the invention can also be present in the form of zwitterions. Particularly preferred pharmaceutically acceptable salts of the invention are the sodium, lithium, potassium and trialkylammonium salts.

The term “protecting group”, alone or in combination, signifies a group which selectively blocks a reactive site in a multifunctional compound such that a chemical reaction can be carried out selectively at another unprotected reactive site. Protecting groups can be removed. Exemplary protecting groups are amino-protecting groups, carboxy-protecting groups or hydroxy-protecting groups.

“Phosphate protecting group” is a protecting group of the phosphate group. Examples of phosphate protecting group are 2-cyanoethyl and methyl. A particular example of phosphate protecting group is 2-cyanoethyl.

“Hydroxyl protecting group” is a protecting group of the hydroxyl group and is also used to protect thiol groups. Examples of hydroxyl protecting groups are acetyl (Ac), benzoyl (Bz), benzyl (Bn), β-methoxyethoxymethyl ether (MEM), dimethoxytrityl (or bis-(4-methoxyphenyl)phenylmethyl) (DMT), trimethoxytrityl (or tris-(4-methoxyphenyl)phenylmethyl) (TMT), methoxymethyl ether (MOM), methoxytrityl [(4-methoxyphenyl)diphenylmethyl (MMT), p-methoxybenzyl ether (PMB), methylthiomethyl ether, pivaloyl (Piv), tetrahydropyranyl (THP), tetrahydrofuran (THF), trityl or triphenylmethyl (Tr), silyl ether (for example trimethylsilyl (TMS), tert-butyldimethylsilyl (TBDMS), tri-iso-propylsilyloxymethyl (TOM) and triisopropylsilyl (TIPS) ethers), methyl ethers and ethoxyethyl ethers (EE). Particular examples of hydroxyl protecting group are DMT and TMT, in particular DMT.

“Thiohydroxyl protecting group” is a protecting group of the thiohydroxyl group. Examples of thiohydroxyl protecting groups are those of the “hydroxyl protecting group”.

If one of the starting materials or compounds of the invention contain one or more functional groups which are not stable or are reactive under the reaction conditions of one or more reaction steps, appropriate protecting groups (as described e.g., in “Protective Groups in Organic Chemistry” by T. W. Greene and P. G. M. Wuts, 3^{rd} Ed., 1999, Wiley, New York) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

The compounds described herein can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides.

Antisense Oligonucleotides

The term “antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides of the present invention are single-stranded. It is understood that single-stranded oligonucleotides of the present invention can form hairpins or intermolecular duplex structures (duplex between two molecules of the same oligonucleotide), as long as the degree of intra or inter self complementarity is less than 50% across of the full length of the oligonucleotide.

Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence, such as a F-G-F′ gapmer region, and may optionally comprise further nucleotide(s), for example a nucleotide linker region which may be used to attach a functional group to the contiguous nucleotide sequence. The nucleotide linker region may or may not be complementary to the target nucleic acid.

Nucleotides

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

Modified Internucleoside Linkage

The term “modified internucleoside linkage” is defined as generally understood by the skilled person as linkages other than phosphodiester (PO) linkages, that covalently couples two nucleosides together. The oligonucleotides of the invention may therefore comprise modified internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides, such as region F and F′.

In an embodiment, the oligonucleotide comprises one or more internucleoside linkages modified from the natural phosphodiester, such one or more modified internucleoside linkages that is for example more resistant to nuclease attack. Nuclease resistance may be determined by incubating the oligonucleotide in blood serum or by using a nuclease resistance assay (e.g., snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 80 or such as at least 90% of the internucleoside linkages in the oligonucleotide, or contiguous nucleotide sequence thereof, are nuclease resistant internucleoside linkages. In some embodiments all of the internucleoside linkages of the oligonucleotide, or contiguous nucleotide sequence thereof, are nuclease resistant internucleoside linkages. It will be recognized that, in some embodiments the nucleosides which link the oligonucleotide of the invention to a non-nucleotide functional group, such as a conjugate, may be phosphodiester.

A preferred modified internucleoside linkage for use in the oligonucleotide of the invention is phosphorothioate.

Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 80% or such as at least 90% of the internucleoside linkages in the oligonucleotide, or contiguous nucleotide sequence thereof, are phosphorothioate. In some embodiments, other than the phosphorotrithioate internucleoside linkages, all of the internucleoside linkages of the oligonucleotide, or contiguous nucleotide sequence thereof, are phosphorothioate. In some embodiments, the oligonucleotide of the invention comprises both phosphorothioate internucleoside linkages and at least one phosphodiester linkage, such as 2, 3 or 4 phosphodiester linkages, in addition to the phosphorotrithioate linkage(s). In a gapmer oligonucleotide, phosphodiester linkages, when present, are suitably not located between contiguous DNA nucleosides in the gap region G.

Nuclease resistant linkages, such as phosphorothioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F′, which the internucleoside linkage in region G may be fully phosphorothioate.

Advantageously, all the internucleoside linkages in the contiguous nucleotide sequence of the oligonucleotide, or all the internucleoside linkages of the oligonucleotide, are phosphorothioate linkages.

It is recognized that, as disclosed in EP2742135, antisense oligonucleotides may comprise other internucleoside linkages (other than phosphodiester and phosphorothioate), for example alkyl phosphonate/methyl phosphonate internucleosides, which according to EP2742135 may for example be tolerated in an otherwise DNA phosphorothioate the gap region.

Stereorandom Phosphorothioate Linkages

Phosphorothioate linkages are internucleoside phosphate linkages where one of the non-bridging oxygens has been substituted with a sulfur. The substitution of one of the non-bridging oxygens with a sulfur introduces a chiral center, and as such within a single phosphorothioate oligonucleotide, each phosphorothioate internucleoside linkage will be either in the S (Sp) or R (Rp) stereoisoforms. Such internucleoside linkages are referred to as “chiral internucleoside linkages”. By comparison, phosphodiester internucleoside linkages are non-chiral as they have two non-terminal oxygen atoms.

The designation of the chirality of a stereocenter is determined by standard Cahn-Ingold-Prelog rules (CIP priority rules) first published in Cahn, R. S.; Ingold, C. K.; Prelog, V. (1966) “Specification of Molecular Chirality” Angewandte Chemie International Edition 5 (4): 385-415. doi: 10.1002/anie. 196603851.

During standard oligonucleotide synthesis the stereoselectivity of the coupling and the following sulfurization is not controlled. For this reason the stereochemistry of each phosphorothioate internucleoside linkages is randomly Sp or Rp, and as such a phosphorothioate oligonucleotide produced by traditional oligonucleotide synthesis actually can exist in as many as 2^{X} different phosphorothioate diastereoisomers, where X is the number of phosphorothioate internucleoside linkages. Such oligonucleotides are referred to as stereorandom phosphorothioate oligonucleotides herein, and do not contain any stereodefined internucleoside linkages. Stereorandom phosphorothioate oligonucleotides are therefore mixtures of individual diastereoisomers originating from the non-stereodefined synthesis. In this context the mixture is defined as up to 2^{X} different phosphorothioate diastereoisomers.

Stereodefined Internucleoside Linkages

A stereodefined internucleoside linkage is a chiral internucleoside linkage having a diastereoisomeric excess for one of its two diastereomeric forms, Rp or Sp.

It should be recognized that stereoselective oligonucleotide synthesis methods used in the art typically provide at least about 90% or at least about 95% diastereoselectivity at each chiral internucleoside linkage, and as such up to about 10%, such as about 5% of oligonucleotide molecules may have the alternative diastereoisomeric form.

In some embodiments the diastereoisomeric ratio of each stereodefined chiral internucleoside linkage is at least about 90:10. In some embodiments the diastereoisomeric ratio of each chiral internucleoside linkage is at least about 95:5.

The stereodefined phosphorothioate linkage is a particular example of stereodefined internucleoside linkage.

Stereodefined Phosphorothioate Linkage

A stereodefined phosphorothioate linkage is a phosphorothioate linkage having a diastereomeric excess for one of its two diastereoisomeric forms, Rp or Sp.

The Rp and Sp configurations of the phosphorothioate internucleoside linkages are presented below:

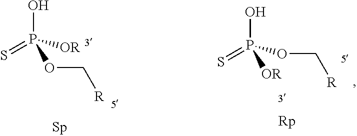

where the 3′ R group represents the 3′ position of the adjacent nucleoside (a 5′ nucleoside), and the 5′ R group represents the 5′ position of the adjacent nucleoside (a 3′ nucleoside).

Rp internucleoside linkages may also be represented as srP, and Sp internucleoside linkages may be represented as ssP herein.

In a particular embodiment, the diastereomeric ratio of each stereodefined phosphorothioate linkage is at least about 90:10 or at least 95:5.

In some embodiments the diastereomeric ratio of each stereodefined phosphorothioate linkage is at least about 97:3. In some embodiments the diastereomeric ratio of each stereodefined phosphorothioate linkage is at least about 98:2. In some embodiments the diastereomeric ratio of each stereodefined phosphorothioate linkage is at least about 99:1.

In some embodiments a stereodefined internucleoside linkage is in the same diastereomeric form (Rp or Sp) in at least 97%, such as at least 98%, such as at least 99%, or (essentially) all of the oligonucleotide molecules present in a population of the oligonucleotide molecule.

Diastereomeric purity can be measured in a model system only having an achiral backbone (i.e. phosphodiesters). It is possible to measure the diastereomeric purity of each monomer by e.g., coupling a monomer having a stereodefine internucleoside linkage to the following model-system “5′ t-po-t-po-t-po 3′”. The result of this will then give: 5′ DMTr-t-srp-t-po-t-po-t-po 3′ or 5′ DMTr-t-ssp-t-po-t-po-t-po 3′ which can be separated using HPLC. The diastereomeric purity is determined by integrating the UV signal from the two possible diastereoisomers and giving a ratio of these e.g., 98:2, 99:1 or >99:1.

It will be understood that the diastereomeric purity of a specific single diastereoisomer (a single stereodefined oligonucleotide molecule) will be a function of the coupling selectivity for the defined stereocenter at each internucleoside position, and the number of stereodefined internucleoside linkages to be introduced. By way of example, if the coupling selectivity at each position is 97%, the resulting purity of the stereodefined oligonucleotide with 15 stereodefined internucleoside linkages will be 0.97¹⁵, i.e. 63% of the desired diastereoisomer as compared to 37% of the other diastereoisomers. The purity of the defined diastereoisomer may after synthesis be improved by purification, for example by HPLC, such as ion exchange chromatography or reverse phase chromatography.

In some embodiments, a stereodefined oligonucleotide refers to a population of an oligonucleotide wherein at least about 40%, such as at least about 50% of the population is of the desired diastereoisomer.

Alternatively stated, in some embodiments, a stereodefined oligonucleotide refers to a population of oligonucleotides wherein at least about 40%, such as at least about 50%, of the population consists of the desired (specific) stereodefined internucleoside linkage motifs (also termed stereodefined motif).

For stereodefined oligonucleotides which comprise both stereorandom and stereodefined internucleoside chiral centers, the purity of the stereodefined oligonucleotide is determined with reference to the % of the population of the oligonucleotide which retains the desired stereodefined internucleoside linkage motif(s), the stereorandom linkages being disregarded in the calculation.

Nucleobase

The term nucleobase includes the purine (e.g., adenine and guanine) and pyrimidine (e.g., uracil, thymine and cytosine) moieties present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol. 45 page 2055 and Bergstrom (2009) Current Protocols in Nucleic Acid Chemistry Suppl. 37 1.4.1.

In some embodiments the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g., A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

Modified Oligonucleotide

The term modified oligonucleotide describes an oligonucleotide comprising one or more sugar-modified nucleosides and/or modified internucleoside linkages. The term chimeric” oligonucleotide is a term that has been used in the literature to describe oligonucleotides with modified nucleosides.

Stereodefined Oligonucleotide

A stereodefined oligonucleotide is an oligonucleotide wherein at least one of the internucleoside linkages is a stereodefined internucleoside linkage.

A stereodefined phosphorothioate oligonucleotide is an oligonucleotide wherein at least one of the internucleoside linkages is a stereodefined phosphorothioate internucleoside linkage.

Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides in a contiguous nucleotide sequence in a nucleic acid molecule (e.g., oligonucleotide) which, at a given position, are complementary to (i.e. form Watson Crick base pairs with) a contiguous nucleotide sequence, at a given position of a separate nucleic acid molecule (e.g., the target nucleic acid). The percentage is calculated by counting the number of aligned bases that form pairs between the two sequences (when aligned with the target sequence 5′-3′ and the oligonucleotide sequence from 3′-5′), dividing by the total number of nucleotides in the oligonucleotide and multiplying by 100. In such a comparison a nucleobase/nucleotide which does not align (form a base pair) is termed a mismatch. Preferably, insertions and deletions are not allowed in the calculation of % complementarity of a contiguous nucleotide sequence.

The term “fully complementary” refers to 100% complementarity.

Identity

The term “identity” as used herein, refers to the number of nucleotides in percent of a contiguous nucleotide sequence in a nucleic acid molecule (e.g., oligonucleotide) which, at a given position, are identical to (i.e. in their ability to form Watson Crick base pairs with the complementary nucleoside) a contiguous nucleotide sequence, at a given position of a separate nucleic acid molecule (e.g., the target nucleic acid). The percentage is calculated by counting the number of aligned bases that are identical between the two sequences dividing by the total number of nucleotides in the oligonucleotide and multiplying by 100. Percent Identity=(Matches×100)/Length of aligned region. Preferably, insertions and deletions are not allowed in the calculation of % complementarity of a contiguous nucleotide sequence.

Hybridization

The term “hybridizing” or “hybridizes” as used herein is to be understood as two nucleic acid strands (e.g., an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RT ln(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

Sugar Modifications

The oligomer of the invention may comprise one or more nucleosides which have a modified sugar moiety, i.e., a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g., by replacement with a hexose ring (HNA), or a bicyclic ring, which typically have a biradical bridge between the C2 and C4 carbons on the ribose ring (LNA), or an unlinked ribose ring which typically lacks a bond between the C2 and C3 carbons (e.g., UNA). Other sugar-modified nucleosides include, for example, bicyclohexose nucleic acids (WO 2011/017521) or tricyclic nucleic acids (WO 2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

2′ Sugar-Modified Nucleosides

A 2′ sugar-modified nucleoside is a nucleoside which has a substituent other than H or —OH at the 2′ position (2′-substituted nucleoside) or comprises a 2′ linked biradical capable of forming a bridge between the 2′ carbon and a second carbon in the ribose ring, such as LNA (2′-4′ biradical bridged) nucleosides.

Indeed, much focus has been spent on developing 2′-substituted nucleosides, and numerous 2′-substituted nucleosides have been found to have beneficial properties when incorporated into oligonucleotides. For example, the 2′-modified sugar may provide enhanced binding affinity and/or increased nuclease resistance to the oligonucleotide. Examples of 2′-substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-fluoro-RNA and 2′-F-ANA nucleoside. Further examples can be found in e.g., Freier & Altmann; Nucl. Acid Res., 1997, 25, 4429-4443 and Uhlmann; Curr. Opinion in Drug Development, 2000, 3(2), 293-213 and Deleavey and Damha, Chemistry and Biology 2012, 19, 937. Below are illustrations of some 2′-substituted modified nucleosides.

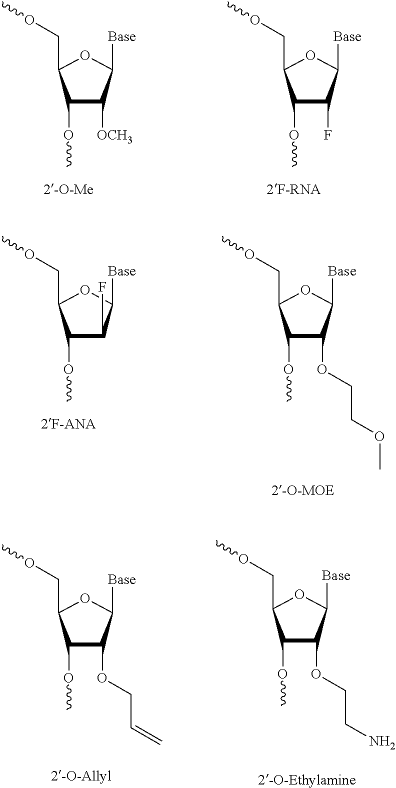

In relation to the present invention 2′-substituted does not include 2′ bridged molecules like LNA.

Locked Nucleic Acid Nucleosides (LNA Nucleosides)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81 and Mitsuoka et al., Nucleic Acids Research 2009, 37(4), 1225-1238.

The 2′-4′ bridge comprises 2 to 4 bridging atoms and is in particular of formula —X—Y—, X being linked to C4′ and Y linked to C2′,

wherein:
X is oxygen, sulfur, —CR^{a}R^{b}—, —C(R^{a})═C(R^{b})—, —C(═CR^{a}R^{b})—, —C(R^{a})═N—, —Si(R^{a})₂—, —SO₂—, —NR^{a}—; —O—NR^{a}—, —NR^{a}—O—, —C(=J)-, Se, —O—NR^{a}—, —NR^{a}—CR^{a}R^{b}—, —N(R^{a})—O— or —O—CR^{a}R^{b}—;
Y is oxygen, sulfur, —(CR^{a}R^{b})ₙ—, —CR^{a}R^{b}—O—CR^{a}R^{b}—, —C(R^{a})═C(R^{b})—, —C(R^{a})═N—, —Si(R^{a})₂—, —SO₂—, —NR^{a}—, —C(=J)-, Se, —O—NR^{a}—, —NR^{a}—CR^{a}R^{b}—, —N(R^{a})—O— or —O—CR^{a}R^{b}—;
with the proviso that —X—Y— is not —O—O—, Si(R^{a})₂—Si(R^{a})₂—, —SO₂—SO₂—, —C(R^{a})═C(R^{b})—C(R^{a})═C(R^{b}), —C(R^{a})═N—C(R^{a})═N—, —C(R^{a})═N—C(R^{a})═C(R^{b}), —C(R^{a})═C(R^{b})—C(R^{a})═N— or —Se—Se—;
J is oxygen, sulfur, ═CH₂ or ═N(R^{a});
R^{a} and R^{b} are independently selected from hydrogen, halogen, hydroxyl, cyano, thiohydroxyl, alkyl, substituted alkyl, alkenyl, substituted alkenyl, alkynyl, substituted alkynyl, alkoxy, substituted alkoxy, alkoxyalkyl, alkenyloxy, carboxyl, alkoxycarbonyl, alkylcarbonyl, formyl, aryl, heterocyclyl, amino, alkylamino, carbamoyl, alkylaminocarbonyl, aminoalkylaminocarbonyl, alkylaminoalkylaminocarbonyl, alkylcarbonylamino, carbamido, alkanoyloxy, sulfonyl, alkylsulfonyloxy, nitro, azido, thiohydroxylsulfidealkylsulfanyl, aryloxycarbonyl, aryloxy, arylcarbonyl, heteroaryl, heteroaryloxycarbonyl, heteroaryloxy, heteroarylcarbonyl, —OC(═X^{a})R^{c}, —OC(═X^{a})NR^{c}R^{d} and —NR^{e}C(═X^{a})NR^{c}R^{d};
or two geminal R^{a} and R^{b} together form optionally substituted methylene;
or two geminal R^{a} and R^{b}, together with the carbon atom to which they are attached, form cycloalkyl or halocycloalkyl, with only one carbon atom of —X—Y—;
wherein substituted alkyl, substituted alkenyl, substituted alkynyl, substituted alkoxy and substituted methylene are alkyl, alkenyl, alkynyl and methylene substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl, formyl, heterocylyl, aryl and heteroaryl; ×^{a} is oxygen, sulfur or —NR^{c};
R^{c}, R^{d} and R^{e} are independently selected from hydrogen and alkyl; and
n is 1, 2 or 3.

In a further particular embodiment of the invention, X is oxygen, sulfur, —NR^{a}—, —CR^{a}R^{b}— or —C(═CR^{a}R^{b})—, particularly oxygen, sulfur, —NH—, —CH₂— or —C(═CH₂)—, more particularly oxygen.

In another particular embodiment of the invention, Y is —CR^{a}R^{b}—, —CR^{a}R^{b}—CR^{a}R^{b}— or —CR^{a}R^{b-}CR^{a}R^{b-}CR^{a}R^{b}—, particularly —CH₂—CHCH₃—, —CHCH₃—CH₂—, —CH₂—CH₂— or —CH₂—CH₂—CH₂—.

In a particular embodiment of the invention, —X—Y— is —O—(CR^{a}R^{b})ₙ—, —S—CR^{a}R^{b}—, —N(R^{a})CR^{a}R^{b}—, —CR^{a}R^{b}—CR^{a}R^{b}—, —O—CR^{a}R^{b}—O—CR^{a}R^{b}—, —CR^{a}R^{b}—O—CR^{a}R^{b}—, —C(═CR^{a}R^{b})—CR^{a}R^{b}—, —N(R^{a})CR^{a}R^{b}—, —O—N(R^{a})—CR^{a}R^{b}— or —N(R^{a})—O—CR^{a}R^{b}—.

In a particular embodiment of the invention, R^{a} and R^{b} are independently selected from the group consisting of hydrogen, halogen, hydroxyl, alkyl and alkoxyalkyl, in particular hydrogen, halogen, alkyl and alkoxyalkyl.

In another embodiment of the invention, R^{a} and R^{b} are independently selected from the group consisting of hydrogen, fluoro, hydroxyl, methyl and —CH₂—O—CH₃, in particular hydrogen, fluoro, methyl and —CH₂—O—CH₃.

Advantageously, one of R^{a} and R^{b} of —X—Y— is as defined above and the other ones are all hydrogen at the same time.

In a further particular embodiment of the invention, R^{a} is hydrogen or alkyl, in particular hydrogen or methyl.

In another particular embodiment of the invention, R^{b} is hydrogen or alkyl, in particular hydrogen or methyl.

In a particular embodiment of the invention, one or both of R^{a} and R^{b} are hydrogen.

In a particular embodiment of the invention, only one of R^{a} and R^{b} is hydrogen.

In one particular embodiment of the invention, one of R^{a} and R^{b} is methyl and the other one is hydrogen.

In a particular embodiment of the invention, R^{a} and R^{b} are both methyl at the same time.

In a particular embodiment of the invention, —X—Y— is —O—CH₂—, —S—CH₂—, —S—CH(CH₃)—, —NH—CH₂—, —O—CH₂CH₂—, —O—CH(CH₂—O—CH₃)—, —O—CH(CH₂CH₃)—, —O—CH(CH₃)—, —O—CH₂₋O—CH₂—, —O—CH₂—O—CH₂—, —CH₂—O—CH₂—, —C(═CH₂)CH₂—, —C(═CH₂)CH(CH₃)—, —N(OCH₃)CH₂— or —N(CH₃)CH₂—;

In a particular embodiment of the invention, —X—Y— is —O—CR^{a}R^{b}— wherein R^{a} and R^{b} are independently selected from the group consisting of hydrogen, alkyl and alkoxyalkyl, in particular hydrogen, methyl and —CH₂—O—CH₃.

In a particular embodiment, —X—Y— is —O—CH₂— or —O—CH(CH₃)—, particularly —O—CH₂—.

The 2′-4′ bridge may be positioned either below the plane of the ribose ring (beta-D-configuration), or above the plane of the ring (alpha-L-configuration), as illustrated in formula (A) and formula (B) respectively.

The LNA nucleoside according to the invention is in particular of formula (B1) or (B2)

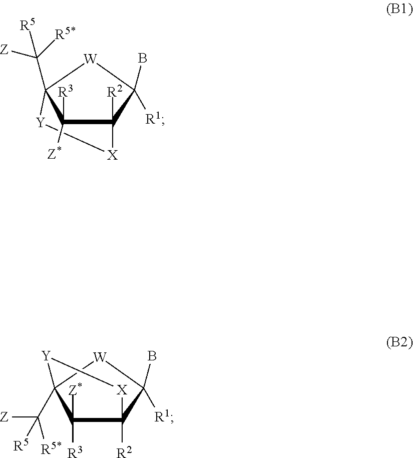

wherein:
W is oxygen, sulfur, —N(R^{a})— or —CR^{a}R^{b}—, in particular oxygen;
B is a nucleobase or a modified nucleobase;
Z is an internucleoside linkage to an adjacent nucleoside or a 5′-terminal group;
Z* is an internucleoside linkage to an adjacent nucleoside or a 3′-terminal group;
R¹, R², R³, R⁵ and R⁵* are independently selected from hydrogen, halogen, alkyl, haloalkyl, alkenyl, alkynyl, hydroxy, alkoxy, alkoxyalkyl, azido, alkenyloxy, carboxyl, alkoxycarbonyl, alkylcarbonyl, formyl and aryl; and
X, Y, R^{a} and R^{b} are as defined above.

In a particular embodiment, in the definition of —X—Y—, R^{a} is hydrogen or alkyl, in particular hydrogen or methyl. In another particular embodiment, in the definition of —X—Y—, R^{b} is hydrogen or alkyl, in particular hydrogen or methyl. In a further particular embodiment, in the definition of —X—Y—, one or both of R^{a} and R^{b} are hydrogen. In a particular embodiment, in the definition of —X—Y—, only one of R^{a} and R^{b} is hydrogen. In one particular embodiment, in the definition of —X—Y—, one of R^{a} and R^{b} is methyl and the other one is hydrogen. In a particular embodiment, in the definition of —X—Y—, R^{a} and R^{b} are both methyl at the same time.

In a further particular embodiment, in the definition of X, R^{a} is hydrogen or alkyl, in particular hydrogen or methyl. In another particular embodiment, in the definition of X, R^{b} is hydrogen or alkyl, in particular hydrogen or methyl. In a particular embodiment, in the definition of X, one or both of R^{a} and R^{b} are hydrogen. In a particular embodiment, in the definition of X, only one of R^{a} and R^{b} is hydrogen. In one particular embodiment, in the definition of X, one of R^{a} and R^{b} is methyl and the other one is hydrogen. In a particular embodiment, in the definition of X, R^{a} and R^{b} are both methyl at the same time.

In a further particular embodiment, in the definition of Y, R^{a} is hydrogen or alkyl, in particular hydrogen or methyl. In another particular embodiment, in the definition of Y, R^{b} is hydrogen or alkyl, in particular hydrogen or methyl. In a particular embodiment, in the definition of Y, one or both of R^{a} and R^{b} are hydrogen. In a particular embodiment, in the definition of Y, only one of R^{a} and R^{b} is hydrogen. In one particular embodiment, in the definition of Y, one of R^{a} and R^{b} is methyl and the other one is hydrogen. In a particular embodiment, in the definition of Y, R^{a} and R^{b} are both methyl at the same time.

In a particular embodiment of the invention R¹, R², R³, R⁵ and R⁵* are independently selected from hydrogen and alkyl, in particular hydrogen and methyl.

In a further particular advantageous embodiment of the invention, R¹, R², R³, R⁵ and R⁵* are all hydrogen at the same time.

In another particular embodiment of the invention, R¹, R², R³, are all hydrogen at the same time, one of R⁵ and R⁵* is hydrogen and the other one is as defined above, in particular alkyl, more particularly methyl.

In a particular embodiment of the invention, R⁵ and R⁵* are independently selected from hydrogen, halogen, alkyl, alkoxyalkyl and azido, in particular from hydrogen, fluoro, methyl, methoxyethyl and azido. In particular, advantageous embodiments of the invention, one of R⁵ and R⁵* is hydrogen and the other one is alkyl, in particular methyl, halogen, in particular fluoro, alkoxyalkyl, in particular methoxyethyl or azido; or R⁵ and R⁵* are both hydrogen or halogen at the same time, in particular both hydrogen of fluoro at the same time. In such particular embodiments, W can advantageously be oxygen, and —X—Y— advantageously —O—CH₂—.

In a particular embodiment of the invention, —X—Y— is —O—CH₂—, W is oxygen and R¹, R², R³, R⁵ and R⁵* are all hydrogen at the same time. Such LNA nucleosides are disclosed in WO 99/014226, WO 00/66604, WO 98/039352 and WO 2004/046160 which are all hereby incorporated by reference, and include what are commonly known in the art as beta-D-oxy LNA and alpha-L-oxy LNA nucleosides.

In another particular embodiment of the invention, —X—Y— is —S—CH₂—, W is oxygen and R¹, R², R³, R⁵ and R⁵* are all hydrogen at the same time. Such thio LNA nucleosides are disclosed in WO 99/014226 and WO 2004/046160 which are hereby incorporated by reference.

In another particular embodiment of the invention, —X—Y— is —NH—CH₂—, W is oxygen and R¹, R², R³, R⁵ and R⁵* are all hydrogen at the same time. Such amino LNA nucleosides are disclosed in WO 99/014226 and WO 2004/046160 which are hereby incorporated by reference.

In another particular embodiment of the invention, —X—Y— is —O—CH₂CH₂— or —OCH₂CH₂CH₂—, W is oxygen, and R¹, R², R³, R⁵ and R⁵* are all hydrogen at the same time. Such LNA nucleosides are disclosed in WO 00/047599 and Morita et al., Bioorganic & Med. Chem. Lett. 12, 73-76, which are hereby incorporated by reference, and include what are commonly known in the art as 2′-O-4′C-ethylene bridged nucleic acids (ENA).

In another particular embodiment of the invention, —X—Y— is —O—CH₂—, W is oxygen, R¹, R², R³ are all hydrogen at the same time, one of R⁵ and R⁵* is hydrogen and the other one is not hydrogen, such as alkyl, for example methyl. Such 5′ substituted LNA nucleosides are disclosed in WO 2007/134181 which is hereby incorporated by reference.

In another particular embodiment of the invention, —X—Y— is —O—CR^{a}R^{b}—, wherein one or both of R^{a} and R^{b} are not hydrogen, in particular alkyl such as methyl, W is oxygen, R¹, R², R³ are all hydrogen at the same time, one of R⁵ and R⁵* is hydrogen and the other one is not hydrogen, in particular alkyl, for example methyl. Such bis modified LNA nucleosides are disclosed in WO 2010/077578 which is hereby incorporated by reference.

In another particular embodiment of the invention, —X—Y— is —O—CHR^{a}—, W is oxygen and R¹, R², R³, R⁵ and R⁵* are all hydrogen at the same time. Such 6′-substituted LNA nucleosides are disclosed in WO 2010/036698 and WO 2007/090071 which are both hereby incorporated by reference. In such 6′-substituted LNA nucleosides, R^{a} is in particular C₁-C₆ alkyl, such as methyl.

In another particular embodiment of the invention, —X—Y— is —O—CH(CH₂—O—CH₃)— (“2′ O-methoxyethyl bicyclic nucleic acid”, Seth el al. J. Org. Chem. 2010, Vol 75(5) pp. 1569-81).

In another particular embodiment of the invention, —X—Y— is —O—CH(CH₂CH₃)—.

In another particular embodiment of the invention, —X—Y— is —O—CH(CH₂—O—CH₃)—, W is oxygen and R¹, R², R³, R⁵ and R⁵* are all hydrogen at the same time. Such LNA nucleosides are also known in the art as cyclic MOEs (cMOE) and are disclosed in WO 2007/090071.

In another particular embodiment of the invention, —X—Y— is —O—CH(CH₃)— (“2′O-ethyl bicyclic nucleic acid”, Seth at al., J. Org. Chem. 2010, Vol 75(5) pp. 1569-81).

In another particular embodiment of the invention, —X—Y— is —O—CH₂₋O—CH₂— (Seth el al., J. Org. Chem 2010 op. cit.)

In another particular embodiment of the invention, —X—Y— is —O—CH(CH₃)—, W is oxygen and R¹, R², R³, R⁵ and R⁵* are all hydrogen at the same time. Such 6′-methyl LNA nucleosides are also known in the art as cET nucleosides, and may be either (S)-cET or (R)-cET diastereoisomers, as disclosed in WO 2007/090071 (beta-D) and WO 2010/036698 (alpha-L) which are both hereby incorporated by reference.

In another particular embodiment of the invention, —X—Y— is —O—CR^{a}R^{b}—, wherein neither R^{a} nor R^{b} is hydrogen, W is oxygen and R¹, R², R³, R⁵ and R⁵* are all hydrogen at the same time. In a particular embodiment, R^{a} and R^{b} are both alkyl at the same time, in particular both methyl at the same time. Such 6′-di-substituted LNA nucleosides are disclosed in WO 2009/006478 which is hereby incorporated by reference.

In another particular embodiment of the invention, —X—Y— is —S—CHR^{a}—, W is oxygen and R¹, R², R³, R⁵ and R⁵* are all hydrogen at the same time. Such 6′-substituted thio LNA nucleosides are disclosed in WO 2011/156202 which is hereby incorporated by reference. In a particular embodiment of such 6′-substituted thio LNA, R^{a} is alkyl, in particular methyl.

In a particular embodiment of the invention, —X—Y— is —C(═CH₂)C(R^{a}R^{b})—, —C(═CHF)C(R^{a}R^{b})— or —C(═CF₂)C(R^{a}R^{b})—, W is oxygen and R¹, R², R³, R⁵ and R⁵ are all hydrogen at the same time. R^{a} and R^{b} are advantageously independently selected from hydrogen, halogen, alkyl and alkoxyalkyl, in particular hydrogen, methyl, fluoro and methoxymethyl. R^{a} and R^{b} are in particular both hydrogen or methyl at the same time or one of R^{a} and R^{b} is hydrogen and the other one is methyl. Such vinyl carbo LNA nucleosides are disclosed in WO 2008/154401 and WO 2009/067647 which are both hereby incorporated by reference.

In a particular embodiment of the invention, —X—Y— is —N(OR^{a})—CH₂—, W is oxygen and R¹, R², R³, R⁵ and R⁵* are all hydrogen at the same time. In a particular embodiment, R^{a} is alkyl such as methyl. Such LNA nucleosides are also known as N substituted LNAs and are disclosed in WO 2008/150729 which is hereby incorporated by reference.

In a particular embodiment of the invention, —X—Y— is —O—N(R^{a})—, —N(R^{a})—O—, —NR^{a}—CR^{a}R^{b}—CR^{a}R^{b}— or —NR^{a}—CR^{a}R^{b}—, W is oxygen and R¹, R², R³, R⁵ and R⁵* are all hydrogen at the same time. R^{a} and R^{b} are advantageously independently selected from hydrogen, halogen, alkyl and alkoxyalkyl, in particular hydrogen, methyl, fluoro and methoxy methyl.

In a particular embodiment, R^{a} is alkyl, such as methyl, R^{b} is hydrogen or methyl, in particular hydrogen (Seth et al., J. Org. Chem 2010 op. cit.).

In a particular embodiment of the invention, —X—Y— is —O—N(CH₃)— (Seth et al., J. Org. Chem 2010 op. cit.).

In a particular embodiment of the invention, R⁵ and R⁵* are both hydrogen at the same time. In another particular embodiment of the invention, one of R⁵ and R⁵* is hydrogen and the other one is alkyl, such as methyl. In such embodiments, R¹, R² and R³ can be in particular hydrogen and —X—Y— can be in particular —O—CH₂— or —O—CHC(R^{a})₃—, such as —O—CH(CH₃)—.

In a particular embodiment of the invention, —X—Y— is —CR^{a}R^{b}—O—CR^{a}R^{b}—, such as —CH₂—O—CH₂—, W is oxygen and R¹, R², R³, R⁵ and R⁵* are all hydrogen at the same time. In such particular embodiments, R^{a} can be in particular alkyl such as methyl, R^{b} hydrogen or methyl, in particular hydrogen. Such LNA nucleosides are also known as conformationally restricted nucleotides (CRNs) and are disclosed in WO 2013/036868 which is hereby incorporated by reference.

In a particular embodiment of the invention, —X—Y— is —O—CR^{a}R^{b}—O—CR^{a}R^{b}—, such as —O—CH₂—O—CH₂—, W is oxygen and R¹, R², R³, R⁵ and R⁵* are all hydrogen at the same time. R^{a} and R^{b} are advantageously independently selected from hydrogen, halogen, alkyl and alkoxyalkyl, in particular hydrogen, methyl, fluoro and methoxymethyl. In such a particular embodiment, R^{a} can be in particular alkyl such as methyl, R^{b} hydrogen or methyl, in particular hydrogen. Such LNA nucleosides are also known as COC nucleotides and are disclosed in Mitsuoka et al., Nucleic Acids Research 2009, 37(4), 1225-1238, which is hereby incorporated by reference.

It will be recognized than, unless specified, the LNA nucleosides may be in the beta-D or alpha-L stereoisoform.

Particular examples of LNA nucleosides of the invention are presented in Scheme 1 (wherein B is as defined above).

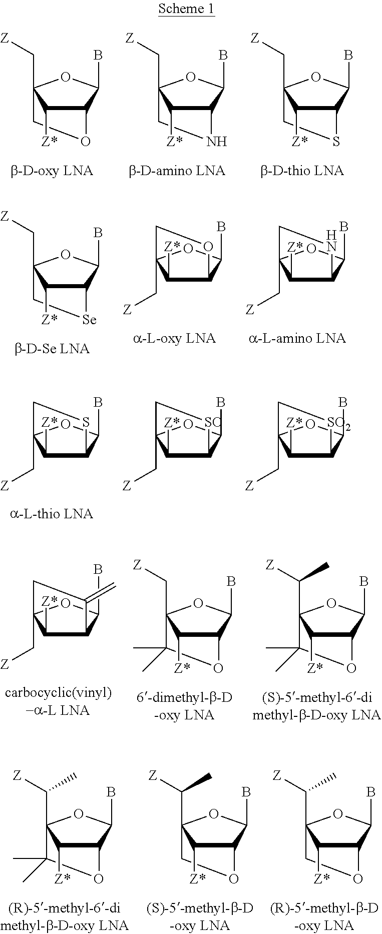

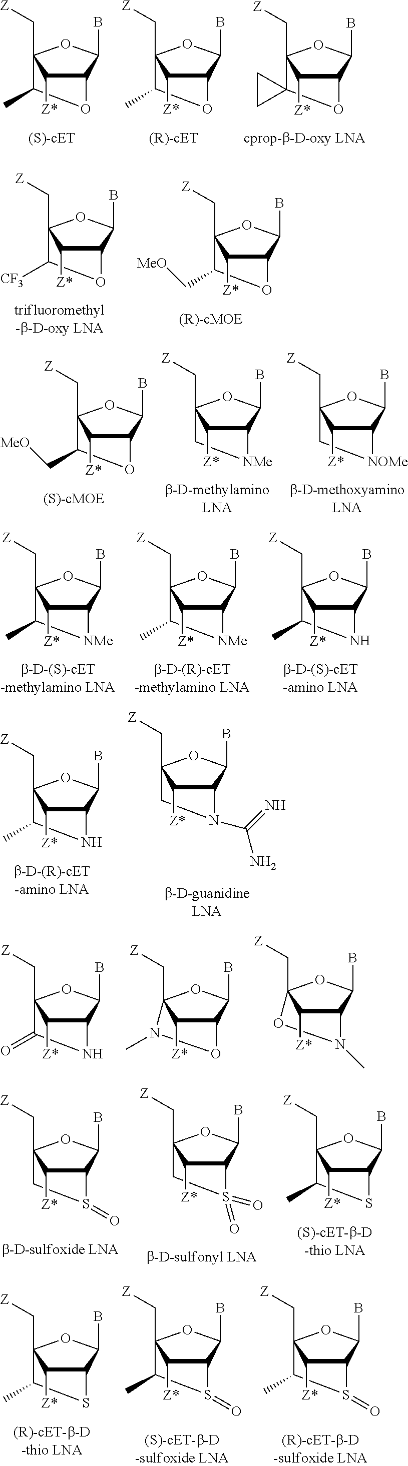

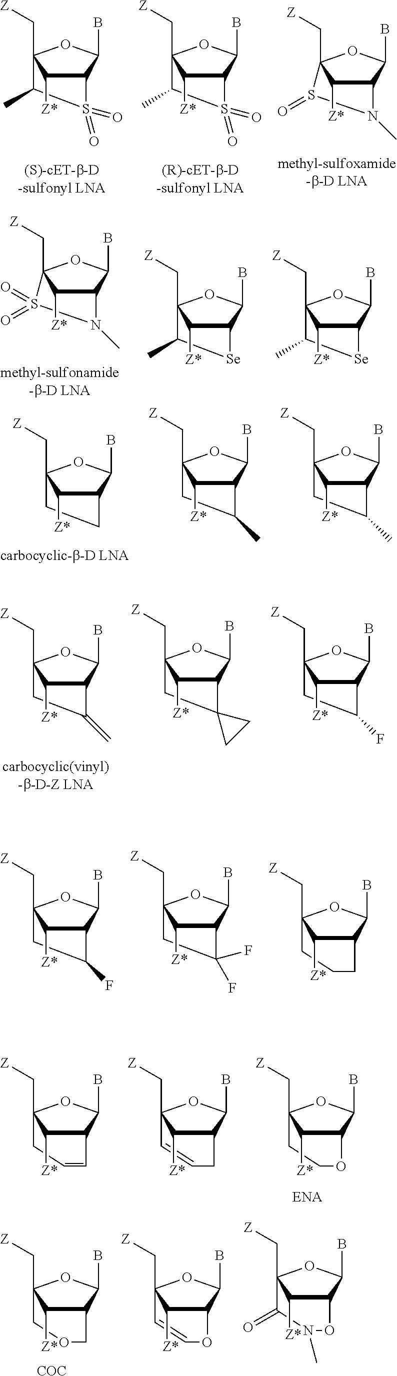

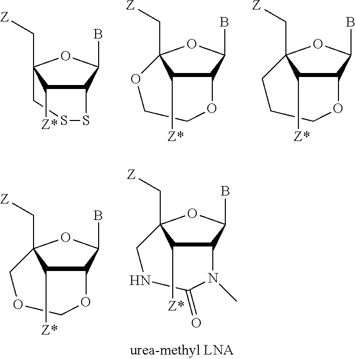

Particular LNA nucleosides are beta-D-oxy-LNA, 6′-methyl-beta-D-oxy LNA such as (S)-6′-methyl-beta-D-oxy-LNA ((S)-cET) and ENA.

RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference). For use in determining RHase H activity, recombinant human RNase H1 is available from Lubio Science GmbH, Lucerne, Switzerland.

Gapmer

The antisense oligonucleotide of the invention, or contiguous nucleotide sequence thereof may be a gapmer. The antisense gapmers are commonly used to inhibit a target nucleic acid via RNase H mediated degradation. A gapmer oligonucleotide comprises at least three distinct structural regions a 5′-flank, a gap and a 3′-flank, F-G-F′ in the ‘5->3’ orientation. The “gap” region (G) comprises a stretch of contiguous DNA nucleotides which enable the oligonucleotide to recruit RNase H. The gap region is flanked by a 5′ flanking region (F) comprising one or more sugar-modified nucleosides, advantageously high affinity sugar-modified nucleosides, and by a 3′ flanking region (F′) comprising one or more sugar-modified nucleosides, advantageously high affinity sugar-modified nucleosides. The one or more sugar-modified nucleosides in region F and F′ enhance the affinity of the oligonucleotide for the target nucleic acid (i.e., are affinity-enhancing sugar-modified nucleosides). In some embodiments, the one or more sugar-modified nucleosides in region F and F′ are 2′ sugar-modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar-modified nucleoside of the 5′ (F) or 3′ (F′) region respectively. The flanks may be further defined by having at least one sugar-modified nucleoside at the end most distant from the gap region, i.e. at the 5′ end of the 5′ flank and at the 3′ end of the 3′ flank.

Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 32 nucleosides, such as 13 to 24, such as 14 to 22 nucleosides, Such as from 14 to 17, such as 16 to 18 nucleosides.

By way of example, the gapmer oligonucleotide of the present invention can be represented by the following formulae:

F₁₋₈-G₅₋₁₆-F′₁₋₈, such as

F₁₋₈-G₇₋₁₆-F′₂₋₈,

with the proviso that the overall length of the gapmer regions F-G-F′ is at least 12, such as at least 14 nucleotides in length.

Regions F, G and F′ are further defined below and can be incorporated into the F-G-F′ formula.

Gapmer—Region G

Region G (gap region) of the gapmer is a region of nucleosides which enables the oligonucleotide to recruit RNaseH, such as human RNase H1, typically DNA nucleosides. RNaseH is a cellular enzyme which recognizes the duplex between DNA and RNA, and enzymatically cleaves the RNA molecule. Suitable gapmers may have a gap region (G) of at least 5 or 6 contiguous DNA nucleosides, such as 5-16 contiguous DNA nucleosides, such as 6-15 contiguous DNA nucleosides, such as 7-14 contiguous DNA nucleosides, such as 8-12 contiguous DNA nucleotides, such as 8-12 contiguous DNA nucleotides in length. The gap region G may, in some embodiments consist of 6, 7, 8, 9, 10, 11, 12, 13, 14, 15 or 16 contiguous DNA nucleosides. Cytosine (C) DNA in the gap region may in some instances be methylated, such residues are either annotated as 5-methyl-cytosine (^{me}C or with an e instead of a c). Methylation of Cytosine DNA in the gap is advantageous if eg dinucleotides are present in the gap to reduce potential toxicity, the modification does not have significant impact on efficacy of the oligonucleotides.

In some embodiments the gap region G may consist of 6, 7, 8, 9, 10, 11, 12, 13, 14, 15 or 16 contiguous phosphorothioate linked DNA nucleosides. In some embodiments, all internucleoside linkages in the gap are phosphorothioate linkages.

Whilst traditional gapmers have a DNA gap region, there are numerous examples of modified nucleosides which allow for RNaseH recruitment when they are used within the gap region. Modified nucleosides which have been reported as being capable of recruiting RNaseH when included within a gap region include, for example, alpha-L-LNA, C4′ alkylated DNA (as described in PCT/EP2009/050349 and Vester et al., Bioorg. Med. Chem. Lett. 18 (2008) 2296-2300, both incorporated herein by reference), arabinose derived nucleosides like ANA and 2′F-ANA (Mangos et al. 2003 J. AM. CHEM. SOC. 125, 654-661), UNA (unlocked nucleic acid) (as described in Fluiter et al., Mol. Biosyst., 2009, 10, 1039 incorporated herein by reference). UNA is unlocked nucleic acid, typically where the bond between C2 and C3 of the ribose has been removed, forming an unlocked “sugar” residue. The modified nucleosides used in such gapmers may be nucleosides which adopt a 2′ endo (DNA like) structure when introduced into the gap region, i.e. modifications which allow for RNaseH recruitment). In some embodiments the DNA Gap region (G) described herein may optionally contain 1 to 3 sugar-modified nucleosides which adopt a 2′ endo (DNA like) structure when introduced into the gap region.

Region G—“Gap-Breaker”

Alternatively, there are numerous reports of the insertion of a modified nucleoside which confers a 3′ endo conformation into the gap region of gapmers, whilst retaining some RNaseH activity. Such gapmers with a gap region comprising one or more 3′endo modified nucleosides are referred to as “gap-breaker” or “gap-disrupted” gapmers, see for example WO2013/022984. Gap-breaker oligonucleotides retain sufficient region of DNA nucleosides within the gap region to allow for RNaseH recruitment. The ability of gapbreaker oligonucleotide design to recruit RNaseH is typically sequence or even compound specific-see Rukov et al. 2015 Nucl. Acids Res. Vol. 43 pp. 8476-8487, which discloses “gapbreaker” oligonucleotides which recruit RNaseH which in some instances provide a more specific cleavage of the target RNA. Modified nucleosides used within the gap region of gap-breaker oligonucleotides may for example be modified nucleosides which confer a 3′endo confirmation, such 2′-O-methyl (OMe) or 2′-O-MOE (MOE) nucleosides, or beta-D LNA nucleosides (the bridge between C2′ and C4′ of the ribose sugar ring of a nucleoside is in the beta conformation), such as beta-D-oxy LNA or ScET nucleosides.

As with gapmers containing region G described above, the gap region of gap-breaker or gap-disrupted gapmers, have a DNA nucleoside at the 5′ end of the gap (adjacent to the 3′ nucleoside of region F), and a DNA nucleoside at the 3′ end of the gap (adjacent to the 5′ nucleoside of region F′). Gapmers which comprise a disrupted gap typically retain a region of at least 3 or 4 contiguous DNA nucleosides at either the 5′ end or 3′ end of the gap region.

Exemplary designs for gap-breaker oligonucleotides include

F₁₋₈-[D₃₋₄-E₁-D₃₋₄]-F′₁₋₈,

F₁₋₈-[D₁₋₄-E₁-D₃₋₄]-F′₁₋₈,

F₁₋₈-[D₃₋₄-E₁-D₁₋₄]-F′₁₋₈,

wherein region G is within the brackets [Dₙ-Eᵣ-Dₘ], D is a contiguous sequence of DNA nucleosides, E is a modified nucleoside (the gap-breaker or gap-disrupting nucleoside), and F and F′ are the flanking regions as defined herein, and with the proviso that the overall length of the gapmer regions F-G-F′ is at least 12, such as at least 14 nucleotides in length.

In some embodiments, region G of a gap disrupted gapmer comprises at least 6 DNA nucleosides, such as 6, 7, 8, 9, 10, 11, 12, 13, 14, 15 or 16 DNA nucleosides. As described above, the DNA nucleosides may be contiguous or may optionally be interspersed with one or more modified nucleosides, with the proviso that the gap region G is capable of mediating RNaseH recruitment.

Gapmer—Flanking Regions, F and F′

Region F is positioned immediately adjacent to the 5′ DNA nucleoside of region G. The 3′ most nucleoside of region F is a sugar-modified nucleoside, such as a high affinity sugar-modified nucleoside, for example a 2′-substituted nucleoside, such as a MOE nucleoside, or an FNA nucleoside.

Region F′ is positioned immediately adjacent to the 3′ DNA nucleoside of region G. The 5′ most nucleoside of region F′ is a sugar-modified nucleoside, such as a high affinity sugar-modified nucleoside, for example a 2′-substituted nucleoside, such as a MOE nucleoside, or an FNA nucleoside.

Region F is 1-8 contiguous nucleotides in length, such as 2-6, such as 3-4 contiguous nucleotides in length. Advantageously the 5′ most nucleoside of region F is a sugar-modified nucleoside. In some embodiments the two 5′ most nucleoside of region F are sugar-modified nucleoside. In some embodiments the 5′ most nucleoside of region F is an FNA nucleoside. In some embodiments the two 5′ most nucleoside of region F are FNA nucleosides. In some embodiments the two 5′ most nucleoside of region F are 2′-substituted nucleoside nucleosides, such as two 3′ MOE nucleosides. In some embodiments the 5′ most nucleoside of region F is a 2′-substituted nucleoside, such as a MOE nucleoside.

Region F′ is 2-8 contiguous nucleotides in length, such as 3-6, such as 4-5 contiguous nucleotides in length. Advantageously, embodiments the 3′ most nucleoside of region F′ is a sugar-modified nucleoside. In some embodiments the two 3′ most nucleoside of region F′ are sugar-modified nucleoside. In some embodiments the two 3′ most nucleoside of region F′ are LNA nucleosides. In some embodiments the 3′ most nucleoside of region F′ is an LNA nucleoside. In some embodiments the two 3′ most nucleoside of region F′ are 2′-substituted nucleoside nucleosides, such as two 3′ MOE nucleosides. In some embodiments the 3′ most nucleoside of region F′ is a 2′-substituted nucleoside, such as a MOE nucleoside.

It should be noted that when the length of region F or F′ is one, it is advantageously an LNA nucleoside.

In some embodiments, region F and F′ independently consists of or comprises a contiguous sequence of sugar-modified nucleosides. In some embodiments, the sugar-modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, region F and F′ independently comprises both LNA and a 2′-substituted modified nucleosides (mixed wing design).

In some embodiments, region F and F′ consists of only one type of sugar-modified nucleosides, such as only MOE or only beta-D-oxy LNA or only ScET. Such designs are also termed uniform flanks or uniform gapmer design.

In some embodiments, all the nucleosides of region F or F′, or F and F′ are LNA nucleosides, such as independently selected from beta-D-oxy LNA, ENA or ScET nucleosides. In some embodiments region F consists of 1-5, such as 2-4, such as 3-4 such as 1, 2, 3, 4 or 5 contiguous LNA nucleosides. In some embodiments, all the nucleosides of region F and F′ are beta-D-oxy LNA nucleosides.

In some embodiments, all the nucleosides of region F or F′, or F and F′ are 2′-substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′-substituted nucleosides, such as OMe or MOE nucleosides. In some embodiments it is the 5′ (F) flanking region that consists of 2′-substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists of 2′-substituted nucleosides, such as OMe or MOE nucleosides whereas the 5′ (F) flanking region comprises at least one LNA nucleoside, such as beta-D-oxy LNA nucleosides or cET nucleosides.

In some embodiments, all the modified nucleosides of region F and F′ are LNA nucleosides, such as independently selected from beta-D-oxy LNA, ENA or ScET nucleosides, wherein region F or F′, or F and F′ may optionally comprise DNA nucleosides (an alternating flank, see definition of these for more details). In some embodiments, all the modified nucleosides of region F and F′ are beta-D-oxy LNA nucleosides, wherein region F or F′, or F and F′ may optionally comprise DNA nucleosides (an alternating flank, see definition of these for more details).

In some embodiments the 5′ most and the 3′ most nucleosides of region F and F′ are LNA nucleosides, such as beta-D-oxy LNA nucleosides or ScET nucleosides.

In some embodiments, the internucleoside linkage between region F and region G is a phosphorothioate internucleoside linkage. In some embodiments, the internucleoside linkage between region F′ and region G is a phosphorothioate internucleoside linkage. In some embodiments, the internucleoside linkages between the nucleosides of region F or F′, F and F′ are phosphorothioate internucleoside linkages.

Further gapmer designs are disclosed in WO 2004/046160, WO 2007/146511 and WO 2008/113832, hereby incorporated by reference.

LNA Gapmer

An LNA gapmer is a gapmer wherein either one or both of region F and F′ 25 comprises or consists of LNA nucleosides. A beta-D-oxy gapmer is a gapmer wherein either one or both of region F and F′ comprises or consists of beta-D-oxy LNA nucleosides.

In some embodiments the LNA gapmer is of formula: [LNA]₁₋₅-[region G]-[LNA]₁₋₅, wherein region G is as defined in the Gapmer region G definition.

MOE Gapmers

A MOE gapmers is a gapmer wherein regions F and F′ consist of MOE nucleosides. In some embodiments the MOE gapmer is of design [MOE]₁₋₈-[Region G]-[MOE]₁₋₈, such as [MOE]₂₋₇-[Region G]₅₋₁₆-[MOE]₂₋₇, such as [MOE]₃₋₆-[Region G]-[MOE]₃₋₆, wherein region G is as defined in the Gapmer definition. MOE gapmers with a 5-10-5 design (MOE-DNA-MOE) have been widely used in the art.

Mixed Wing Gapmer

A mixed wing gapmer is an LNA gapmer wherein one or both of region F and F′ comprise a 2′-substituted nucleoside, such as a 2′-substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as a MOE nucleoside. In some embodiments wherein at least one of region F and F′, or both region F and F′ comprise at least one ENA nucleoside, the remaining nucleosides of region F and F′ are independently selected from the group consisting of MOE and ENA. In some embodiments wherein at least one of region F and F′, or both region F and F′ comprise at least two ENA nucleosides, the remaining nucleosides of region F and F′ are independently selected from the group consisting of MOE and ENA. In some mixed wing embodiments, one or both of region F and F′ may further comprise one or more DNA nucleosides.

Mixed wing gapmer designs are disclosed in WO 2008/049085 and WO 2012/109395, both of which are hereby incorporated by reference.

Alternating Flank Gapmers

Flanking regions may comprise both ENA and DNA nucleoside and are referred to as “alternating flanks” as they comprise an alternating motif of FNA-DNA-FNA nucleosides. Gapmers comprising such alternating flanks are referred to as “alternating flank gapmers”. “Alternative flank gapmers” are thus ENA gapmer oligonucleotides where at least one of the flanks (F or F′) comprises DNA in addition to the ENA nucleoside(s). In some embodiments at least one of region F or F′, or both region F and F′, comprise both ENA nucleosides and DNA nucleosides. In such embodiments, the flanking region F or F′, or both F and F′ comprise at least three nucleosides, wherein the 5′ and 3′ most nucleosides of the F and/or F′ region are ENA nucleosides.

Alternating flank ENA gapmers are disclosed in WO 2016/127002.

An alternating flank region may comprise up to 3 contiguous DNA nucleosides, such as 1 to 2 or 1 or 2 or 3 contiguous DNA nucleosides.

The alternating flak can be annotated as a series of integers, representing a number of LNA nucleosides (L) followed by a number of DNA nucleosides (D), for example

[L]₁₋₃-[D]₁₋₄-[L]₁₋₃,

[L]₁₋₂-[D]₁₋₂-[L]₁₋₂-[D]₁₋₂-[L]₁₋₂.

In oligonucleotide designs these will often be represented as numbers such that 2-2-1 represents 5′ [L]₂-[D]₂-[L] 3′, and 1-1-1-1-1 represents 5′ [L]-[D]-[L]-[D]-[L] 3′. The length of the flank (region F and F′) in oligonucleotides with alternating flanks may independently be 3 to 10 nucleosides, such as 4 to 8, such as 5 to 6 nucleosides, such as 4, 5, 6 or 7 modified nucleosides. In some embodiments only one of the flanks in the gapmer oligonucleotide is alternating while the other is constituted of LNA nucleotides. It may be advantageous to have at least two LNA nucleosides at the 3′ end of the 3′ flank (F′), to confer additional exonuclease resistance. Some examples of oligonucleotides with alternating flanks are:

[L]₁₋₅-[D]₁₋₄-[L]₁₋₃-[G]₅₋₁₆-[L]₂₋₆,

[L]₁₋₂-[D]₁₋₂-[L]₁₋₂-[D]₁₋₂-[L]₁₋₂-[G]₅₋₁₆-[L]₁₋₂-[D]₁₋₃-[L]₂₋₄,

[L]₁₋₅-[G]₅₋₁₆-[L]-[D]-[L]-[D]-[L]₂,

with the proviso that the overall length of the gapmer is at least 12, such as at least 14 nucleotides in length.

Region D′ or D″ in an oligonucleotide

The oligonucleotide of the invention may in some embodiments comprise or consist of the contiguous nucleotide sequence of the oligonucleotide which is complementary to the target nucleic acid, such as the gapmer F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively, it may be used to provide exonuclease protection or for ease of synthesis or manufacture.

Region D′ and D″ can be attached to the 5′ end of region F or the 3′ end of region F′, respectively to generate designs of the following formulas D′-F-G-F′, F-G-F′-D″ or D′-F-G-F′-D″. In this instance the F-G-F′ is the gapmer portion of the oligonucleotide and region D′ or D″ constitute a separate part of the oligonucleotide.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D″ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO 2014/076195, which include by way of example a phosphodiester linked DNA dinucleotide. The use of biocleavable linkers in poly-oligonucleotide constructs is disclosed in WO 2015/113922, where they are used to link multiple antisense constructs (e.g., gapmer regions) within a single oligonucleotide.

In one embodiment the oligonucleotide of the invention comprises a region D′ and/or D″ in addition to the contiguous nucleotide sequence which constitutes the gapmer.

In some embodiments, the oligonucleotide of the present invention can be represented by the following formulae:

F-G-F′, in particular F₁₋₈-G₅₋₁₆-F′₂₋₈,

D′-F-G-F′, in particular D′₁₋₃-F₁₋₈-G₅₋₁₆-F′₂₋₈,

F-G-F′-D″, in particular F₁₋₈-G₅₋₁₆-F′₂₋₈-D″₁₋₃,

D′-F-G-F′-D″, in particular D′₁₋₃-F₁₋₈-G₅₋₁₆-F′₂₋₈-D″₁₋₃.

In some embodiments the internucleoside linkage positioned between region D′ and region F is a phosphodiester linkage. In some embodiments the internucleoside linkage positioned between region F′ and region D″ is a phosphodiester linkage.

Totalmers

In some embodiments, all of the nucleosides of the oligonucleotide, or contiguous nucleotide sequence thereof, are sugar-modified nucleosides. Such oligonucleotides are referred to as a totalmers herein.

In some embodiments all of the sugar-modified nucleosides of a totalmer comprise the same sugar modification, for example they may all be LNA nucleosides, or may all be 2′O-MOE nucleosides. In some embodiments the sugar-modified nucleosides of a totalmer may be independently selected from LNA nucleosides and 2′-substituted nucleosides, such as 2′-substituted nucleoside selected from the group consisting of 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments the oligonucleotide comprises both FNA nucleosides and 2′-substituted nucleosides, such as 2′-substituted nucleoside selected from the group consisting of 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments, the oligonucleotide comprises FNA nucleosides and 2′-O-MOE nucleosides. In some embodiments, the oligonucleotide comprises (S)cET FNA nucleosides and 2′-O-MOE nucleosides. In some embodiments, each nucleoside unit of the oligonucleotide is a 2′substituted nucleoside. In some embodiments, each nucleoside unit of the oligonucleotide is a 2′-O-MOE nucleoside.

In some embodiments, all of the nucleosides of the oligonucleotide or contiguous nucleotide sequence thereof are FNA nucleosides, such as beta-D-oxy-FNA nucleosides and/or (S)cET nucleosides. In some embodiments such FNA totalmer oligonucleotides are between 7-12 nucleosides in length (see for example, WO 2009/043353). Such short fully FNA oligonucleotides are particularly effective in inhibiting microRNAs.

Various totalmer compounds are highly effective as therapeutic oligomers, particularly when targeting microRNA (antimiRs) or as splice switching oligomers (SSOs).

In some embodiments, the totalmer comprises or consists of at least one XYX or YXY sequence motif, such as a repeated sequence XYX or YXY, wherein X is FNA and Y is an alternative (i.e. non FNA) nucleotide analogue, such as a 2′-OMe RNA unit and 2′-fluoro DNA unit. The above sequence motif may, in some embodiments, be XXY, XYX, YXY or YYX for example.

In some embodiments, the totalmer may comprise or consist of a contiguous nucleotide sequence of between 7 and 24 nucleotides, such as 7, 9, 10, 11, 12, 13, 14, 15, 16, 17, 18, 19, 20, 21, 22 or 23 nucleotides.

In some embodiments, the contiguous nucleotide sequence of the totolmer comprises of at least 30%, such as at least 40%, such as at least 50%, such as at least 60%, such as at least 70%, such as at least 80%, such as at least 90%, such as 95%, such as 100% FNA units. For full FNA compounds, it is advantageous that they are less than 12 nucleotides in length, such as 7-10.

The remaining units may be selected from the non-FNA nucleotide analogues referred to herein in, such those selected from the group consisting of 2′-O-alkyl-RNA unit, 2′-OMe-RNA unit, 2′-amino-DNA unit, 2′-fluoro-DNA unit, FNA unit, PNA unit, HNA unit, IN A unit, and a 2′MOE RNA unit, or the group 2′-OMe RNA unit and 2′-fluoro DNA unit.

Mixmers

The term ‘mixmer’ refers to oligomers which comprise both DNA nucleosides and sugar-modified nucleosides, wherein there are insufficient length of contiguous DNA nucleosides to recruit RNaseH. Suitable mixmers may comprise up to 3 or up to 4 contiguous DNA nucleosides. In some embodiments the mixmers comprise alternating regions of sugar-modified nucleosides, and DNA nucleosides. By alternating regions of sugar-modified nucleosides which form a RNA like (3′endo) conformation when incorporated into the oligonucleotide, with short regions of DNA nucleosides, non-RNaseH recruiting oligonucleotides may be made. Advantageously, the sugar-modified nucleosides are affinity enhancing sugar-modified nucleosides.

Oligonucleotide mixmers are often used to provide occupation based modulation of target genes, such as splice modulators or microRNA inhibitors.

In some embodiments the sugar-modified nucleosides in the mixmer, or contiguous nucleotide sequence thereof, comprise or are all LNA nucleosides, such as (S)cET or beta-D-oxy LNA nucleosides.

In some embodiments all of the sugar-modified nucleosides of a mixmer comprise the same sugar modification, for example they may all be LNA nucleosides, or may all be 2′O-MOE nucleosides. In some embodiments the sugar-modified nucleosides of a mixmer may be independently selected from LNA nucleosides and 2′-substituted nucleosides, such as 2′-substituted nucleoside selected from the group consisting of 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-L-ANA nucleosides. In some embodiments the oligonucleotide comprises both LNA nucleosides and 2′-substituted nucleosides, such as 2′-substituted nucleoside selected from the group consisting of 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-L-ANA nucleosides. In some embodiments, the oligonucleotide comprises LNA nucleosides and 2′-O-MOE nucleosides. In some embodiments, the oligonucleotide comprises (S)cET LNA nucleosides and 2′-O-MOE nucleosides.

In some embodiments the mixmer, or contiguous nucleotide sequence thereof, comprises only LNA and DNA nucleosides, such LNA mixmer oligonucleotides which may for example be between 8-24 nucleosides in length (see for example, WO2007112754, which discloses LNA antmiR inhibitors of microRNAs).

Various mixmer compounds are highly effective as therapeutic oligomers, particularly when targeting microRNA (antimiRs) or as splice switching oligomers (SSOs).

In some embodiments, the mixmer comprises a motif:

. . . [L]m[D]n[L]m[D]n[L]m . . . or

. . . [L]m[D]n[L]m[D]n[L]m[D]n[L]m . . . or

. . . [L]m[D]n[L]m[D]n[L]m[D]n[L]m[D]n[L]m . . . or

. . . [L]m[D]n[L]m[D]n[L]m[D]n[L]m[D]n[L]m[D]n[L]m . . .

wherein L represents sugar-modified nucleoside such as a LNA or 2′-substituted nucleoside (e.g., 2′-O-MOE), D represents DNA nucleoside, and wherein each m is independently selected from 1-6, and each n is independently selected from 1, 2, 3 and 4, such as 1-3. In some embodiments each L is a LNA nucleoside. In some embodiments, at least one L is a LNA nucleoside and at least one L is a 2′-O-MOE nucleoside. In some embodiments, each L is independently selected from LNA and 2′-O-MOE nucleoside.

In some embodiments, the mixmer may comprise or consist of a contiguous nucleotide sequence of between 10 and 24 nucleotides, such as 11, 12, 13, 14, 15, 16, 17, 18, 19, 20, 21, 22 or 23 nucleotides.

In some embodiments, the contiguous nucleotide sequence of the mixmer comprises of at least 30%, such as at least 40%, such as at least 50% LNA units.

In some embodiments, the mixmer comprises or consists of a contiguous nucleotide sequence of repeating pattern of nucleotide analogues and naturally occurring nucleotides, or one type of nucleotide analogue and a second type of nucleotide analogue. The repeating pattern, may, for instance be: every second or every third nucleotide is a nucleotide analogue, such as LNA, and the remaining nucleotides are naturally occurring nucleotides, such as DNA, or are a 2′-substituted nucleotide analogue such as 2′MOE of 2′fluoro analogues as referred to herein, or, in some embodiments selected form the groups of nucleotide analogues referred to herein. It is recognised that the repeating pattern of nucleotide analogues, such as LNA units, may be combined with nucleotide analogues at fixed positions—e.g., at the 5′ or 3′ termini.

In some embodiments the first nucleotide of the oligomer, counting from the 3′ end, is a nucleotide analogue, such as a LNA nucleotide or a 2′-O-MOE nucleoside.

In some embodiments, which maybe the same or different, the second nucleotide of the oligomer, counting from the 3′ end, is a nucleotide analogue, such as a LNA nucleotide or a 2′-O-MOE nucleoside.

In some embodiments, which maybe the same or different, the 5′ terminal of the oligomer is a nucleotide analogue, such as a LNA nucleotide or a 2′-O-MOE nucleoside.

In some embodiments, the mixmer comprises at least a region comprising at least two consecutive nucleotide analogue units, such as at least two consecutive LNA units.

In some embodiments, the mixmer comprises at least a region comprising at least three consecutive nucleotide analogue units, such as at least three consecutive LNA units.

Conjugate

The term conjugate as used herein refers to an oligonucleotide which is covalently linked to a non-nucleotide moiety (conjugate moiety or region C or third region).

Conjugation of the oligonucleotide of the invention to one or more non-nucleotide moieties may improve the pharmacology of the oligonucleotide, e.g., by affecting the activity, cellular distribution, cellular uptake or stability of the oligonucleotide. In some embodiments the conjugate moiety modifies or enhances the pharmacokinetic properties of the oligonucleotide by improving cellular distribution, bioavailability, metabolism, excretion, permeability, and/or cellular uptake of the oligonucleotide. In particular, the conjugate may target the oligonucleotide to a specific organ, tissue or cell type and thereby enhance the effectiveness of the oligonucleotide in that organ, tissue or cell type. At the same time the conjugate may serve to reduce activity of the oligonucleotide in non-target cell types, tissues or organs, e.g., off target activity or activity in non-target cell types, tissues or organs.

WO 93/07883 and WO 2013/033230 provides suitable conjugate moieties, which are hereby incorporated by reference. Lurther suitable conjugate moieties are those capable of binding to the asialoglycoprotein receptor (ASGPR). In particular, tri-valent N-acetylgalactosamine conjugate moieties are suitable for binding to the ASGPR, see for example WO 2014/076196, WO 2014/207232 and WO 2014/179620 (hereby incorporated by reference). Such conjugates serve to enhance uptake of the oligonucleotide to the liver while reducing its presence in the kidney, thereby increasing the liver/kidney ratio of a conjugated oligonucleotide compared to the unconjugated version of the same oligonucleotide.

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103, each of which is incorporated herein by reference in its entirety.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g., bacterial toxins), vitamins, viral proteins (e.g., capsids) or combinations thereof.

Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g., linker or tether). Linkers serve to covalently connect a third region, e.g., a conjugate moiety (Region C), to a first region, e.g., an oligonucleotide or contiguous nucleotide sequence complementary to the target nucleic acid (region A).

In some embodiments of the invention the conjugate or oligonucleotide conjugate of the invention may optionally, comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In a preferred embodiment the nuclease susceptible linker comprises between 1 and 10 nucleosides, such as 1, 2, 3, 4, 5, 6, 7, 8, 9 or 10 nucleosides, more preferably between 2 and 6 nucleosides and most preferably between 2 and 4 linked nucleosides comprising at least two consecutive phosphodiester linkages, such as at least 3 or 4 or 5 consecutive phosphodiester linkages. Preferably the nucleosides are DNA or RNA. Phosphodiester containing biocleavable linkers are described in more detail in WO 2014/076195 (hereby incorporated by reference).

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups The oligonucleotide conjugates of the present invention can be constructed of the following regional elements A-C, A-B-C, A-B-Y-C, A-Y-B-C or A-Y-C. In some embodiments the linker (region Y) is an amino alkyl, such as a C2-C36 amino alkyl group, including, for example C6 to C12 amino alkyl groups. In a preferred embodiment the linker (region Y) is a C6 amino alkyl group.

The invention thus relates in particular to:

An oligonucleotide according to the invention wherein one of (A¹) and (A²) is a sugar-modified nucleoside and the other one is a DNA;

An oligonucleotide according to the invention wherein (A¹) and (A²) are both a sugar-modified nucleoside at the same time;

An oligonucleotide according to the invention wherein the sugar-modified nucleoside is independently a 2′ sugar-modified nucleoside;

An oligonucleotide according to the invention wherein the 2′ sugar-modified nucleoside is independently selected from is 2′-alkoxy-RNA, in particular 2′-methoxy-RNA, 2′-alkoxyalkoxy-RNA, in particular 2′-methoxyethoxy-RNA, 2′-amino-DNA, 2′-fluoro-RNA or 2′-fluoro-ANA;

An oligonucleotide according to the invention wherein the 2′ sugar-modified nucleoside is 2′-alkoxyalkoxy-RNA, in particular 2′-methoxyethoxy-RNA;

An oligonucleotide according to the invention wherein the 2′ sugar-modified nucleoside is a LNA nucleoside;

An oligonucleotide according to the invention wherein the LNA nucleoside is independently selected from beta-D-oxy LNA, 6′-methyl-beta-D-oxy LNA and ENA, in particular beta-D-oxy LNA;

An oligonucleotide according to the invention comprising further internucleoside linkages selected from phosphodiester internucleoside linkage, phosphorothioate internucleoside linkage and internucleoside linkage as defined in formula (I);

An oligonucleotide according to the invention comprising further internucleoside linkages selected from phosphorothioate internucleoside linkage and internucleoside linkage as defined in formula (I);

An oligonucleotide according to the invention comprising between 1 and 15, in particular between 1 and 5, more particularly 1, 2, 3, 4 or 5 dinucleosides of formula (I) as defined in formula (I);

An oligonucleotide according to the invention wherein the further internucleoside linkages are all phosphorothioate internucleoside linkages of formula —P(═S)(OR)O₂—, wherein R is hydrogen or a phosphate protecting group;

An oligonucleotide according to the invention comprising further nucleosides selected from DNA nucleoside, RNA nucleoside and sugar-modified nucleosides;

An oligonucleotide according to the invention wherein one or more nucleoside is a nucleobase modified nucleoside, such as a nucleoside comprising a 5-methyl cytosine nucleobase;

An oligonucleotide according to the invention wherein the at least one dinucleoside of formula (I) is in the flanking region of the antisense gapmer oligonucleotide or is located between the gap region and the flanking region of the antisense gapmer oligonucleotide, i.e. (A¹) and (A²) are both a sugar-modified nucleoside at the same time or one of (A¹) and (A²) is a DNA nucleoside or a RNA nucleoside and the other one is a sugar-modified nucleoside;

An oligonucleotide according to the invention wherein the gapmer oligonucleotide is a LNA gapmer, a mixed wing gapmer or a 2′-substituted gapmer, in particular a 2′-O-methoxyethyl gapmer;

An oligonucleotide according to the invention wherein A is sulfur.

An oligonucleotide according to the invention wherein the antisense gapmer oligonucleotide comprises a contiguous nucleotide sequence of formula 5′-F-G-F′-3′, wherein G is a region of 5 to 18 nucleosides which is capable of recruiting RNaseH, and said region G is flanked 5′ and 3′ by flanking regions F and F′ respectively, wherein regions F and F′ independently comprise or consist of 1 to 7 2′-sugar-modified nucleotides, wherein the nucleoside of region F which is adjacent to region G is a 2′-sugar-modified nucleoside and wherein the nucleoside of region F′ which is adjacent to region G is a 2′-sugar-modified nucleoside;

An oligonucleotide according to the invention wherein said at least one dinucleoside of formula (I) is positioned in region F or F′, or between region G and region F, or between region G and region F′;

An oligonucleotide according to the invention wherein the 2′-sugar-modified nucleosides in region F or region F′, or in both regions F and F′, are independently selected from 2′-alkoxy-RNA, in particular 2′-methoxy-RNA, 2′-alkoxyalkoxy-RNA, in particular 2′-methoxyethoxy-RNA, 2′-amino-DNA, 2′-fluoro-RNA, 2′-fluoro-ANA and LNA nucleosides;

An oligonucleotide according to the invention wherein all the 2′-sugar-modified nucleosides in region F or region F′, or in both regions F and F′, are LNA nucleosides;

An oligonucleotide according to the invention wherein the 2′-sugar-modified nucleosides in region F or region F′, or in both regions F and F′, are all 2′-alkoxy-RNA, in particular 2′-methoxy-RNA, all 2′-alkoxyalkoxy-RNA, in particular 2′-methoxy ethoxy-RNA, all 2′-amino-DNA, all 2′-fluoro-RNA, all 2′-fluoro-ANA or all LNA nucleosides;

An oligonucleotide according to the invention wherein region F or region F′, or both regions F and F′, comprise at least one LNA nucleoside and at least one DNA nucleoside;

An oligonucleotide according to the invention wherein region F or region F′, or both region F and F′ comprise at least one LNA nucleoside and at least one non-LNA 2′-sugar-modified nucleoside, such as at least one 2′-methoxyethoxy-RNA nucleoside;

An oligonucleotide according to the invention wherein the gap region G comprises 5 to 16, in particular 8 to 16, more particularly 8, 9, 10, 11, 12, 13 or 14 contiguous DNA nucleosides;

An oligonucleotide according to the invention wherein region F and region F′ are independently 1, 2, 3, 4, 5, 6, 7 or 8 nucleosides in length;

An oligonucleotide according to the invention wherein region F and region F′ each independently comprise 1, 2, 3 or 4 LNA nucleosides;

An oligonucleotide according to the invention wherein the LNA nucleosides are independently selected from beta-D-oxy LNA, 6′-methyl-beta-D-oxy LNA and ENA;

An oligonucleotide according to the invention wherein the LNA nucleosides are beta-D-oxy LNA;

An oligonucleotide according to the invention wherein the oligonucleotide, or contiguous nucleotide sequence thereof (F-G-F′), is of 10 to 30 nucleotides in length, in particular 12 to 22, more particularly of 14 to 20 oligonucleotides in length;

An oligonucleotide according to the invention wherein the gapmer oligonucleotide comprises a contiguous nucleotide sequence of formula 5′-D′-F-G-F′-D″-3′, wherein F, G and F′ are as defined in any one of claims 17 to 28 and wherein region D′ and D″ each independently consist of 0 to 5 nucleotides, in particular 2, 3 or 4 nucleotides, in particular DNA nucleotides such as phosphodiester linked DNA nucleosides;

An oligonucleotide according to any one of claims 17 to 29, wherein each flanking region F and F′ independently comprises 1, 2, 3, 4, 5, 6 or 7, in particular one, dinucleoside of formula (I);

An oligonucleotide according to the invention comprising in total one dinucleoside of formula (I), and in particular one dinucleoside of formula (I) positioned in region F′ or between region G and region F′;

An oligonucleotide according to the invention wherein the oligonucleotide is capable of recruiting human RNaseH1;

A pharmaceutically acceptable salt of an oligonucleotide according to the invention, in particular a sodium, a potassium salt or an ammonium salt;

A conjugate comprising an oligonucleotide or a pharmaceutically acceptable salt according to the invention and at least one conjugate moiety covalently attached to said oligonucleotide or said pharmaceutically acceptable salt, optionally via a linker moiety;

A pharmaceutical composition comprising an oligonucleotide, a pharmaceutically acceptable salt or a conjugate according to the invention and a therapeutically inert carrier; and

An oligonucleotide, pharmaceutically acceptable salt or conjugate according to the invention for use as therapeutically active substance.

The invention relates in particular to a compound of formula (I-a)

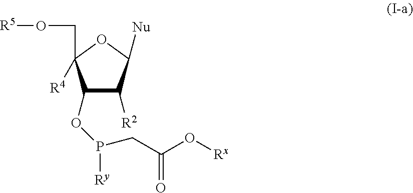

wherein
R² is alkoxy, alkoxyalkoxy or amino; and
R⁴ is hydrogen; or
R⁴ and R² together form X-Y;
   X is oxygen, sulfur, —CR^{a}R^{b}—, —C(R^{a})═C(R^{b})—, —C(═CR^{a}R^{b})—, —C(R^{a})═N—, —Si(R^{a})₂—, —SO₂—, —NR^{a}—; —O—NR^{a}—, —NR^{a}—O—, —C(=J)-, Se, —O—NR^{a}—, —NR^{a}—CR^{a}R^{b}—, —N(R^{a})—O— or —O—CR^{a}R^{b}—;
Y is oxygen, sulfur, —(CR^{a}R^{b})ₙ—, —CR^{a}R^{b}—O—CR^{a}R^{b}—, —C(R^{a})═C(R^{b})—, —C(R^{a})═N—, —Si(R^{a})₂—, —SO₂—, —NR^{a}—, —C(=J)-, Se, —O—NR^{a}—, —NR^{a}—CR^{a}R^{b}—, —N(R^{a})—O— or —O—CR^{a}R^{b}—;
with the proviso that —X—Y— is not —O—O—, Si(R^{a})₂—Si(R^{a})₂—, —SO₂—SO₂—, —C(R^{a})═C(R^{b})—C(R^{a})═C(R^{b}), —C(R^{a})═N—C(R^{a})═N—, —C(R^{a})═N—C(R^{a})═C(R^{b}), —C(R^{a})═C(R^{b})—C(R^{a})═N— or —Se—Se—;
J is oxygen, sulfur, ═CH₂ or ═N(R^{a});
R^{a} and R^{b} are independently selected from hydrogen, halogen, hydroxyl, cyano, thiohydroxyl, alkyl, substituted alkyl, alkenyl, substituted alkenyl, alkynyl, substituted alkynyl, alkoxy, substituted alkoxy, alkoxyalkyl, alkenyloxy, carboxyl, alkoxycarbonyl, alkylcarbonyl, formyl, aryl, heterocyclyl, amino, alkylamino, carbamoyl, alkylaminocarbonyl, aminoalkylaminocarbonyl, alkylaminoalkylaminocarbonyl, alkylcarbonylamino, carbamido, alkanoyloxy, sulfonyl, alkylsulfonyloxy, nitro, azido, thiohydroxylsulfidealkylsulfanyl, aryloxycarbonyl, aryloxy, arylcarbonyl, heteroaryl, heteroaryloxycarbonyl, heteroaryloxy, heteroarylcarbonyl, —OC(═X^{a})R^{c}, —OC(═X^{a})NR^{c}R^{d} and —NR^{e}C(═X^{a})NR^{c}R^{d};
or two geminal R^{a} and R^{b} together form optionally substituted methylene;
or two geminal R^{a} and R^{b}, together with the carbon atom to which they are attached, form cycloalkyl or halocycloalkyl, with only one carbon atom of —X—Y—;
wherein substituted alkyl, substituted alkenyl, substituted alkynyl, substituted alkoxy and substituted methylene are alkyl, alkenyl, alkynyl and methylene substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl, formyl, heterocylyl, aryl and heteroaryl;
X^{a} is oxygen, sulfur or —NR^{c};
R^{c}, R^{d} and R^{e} are independently selected from hydrogen and alkyl;
R⁵ is a hydroxyl protecting group;
R^{x} is cyanoalkyl or alkyl;
R^{y} is dialkylamino or pyrrolidinyl;
Nu is a nucleobase or a protected nucleobase; and n is 1, 2 or 3.

The oligonucleotide according to the invention can for example be prepared according to the following schemes.

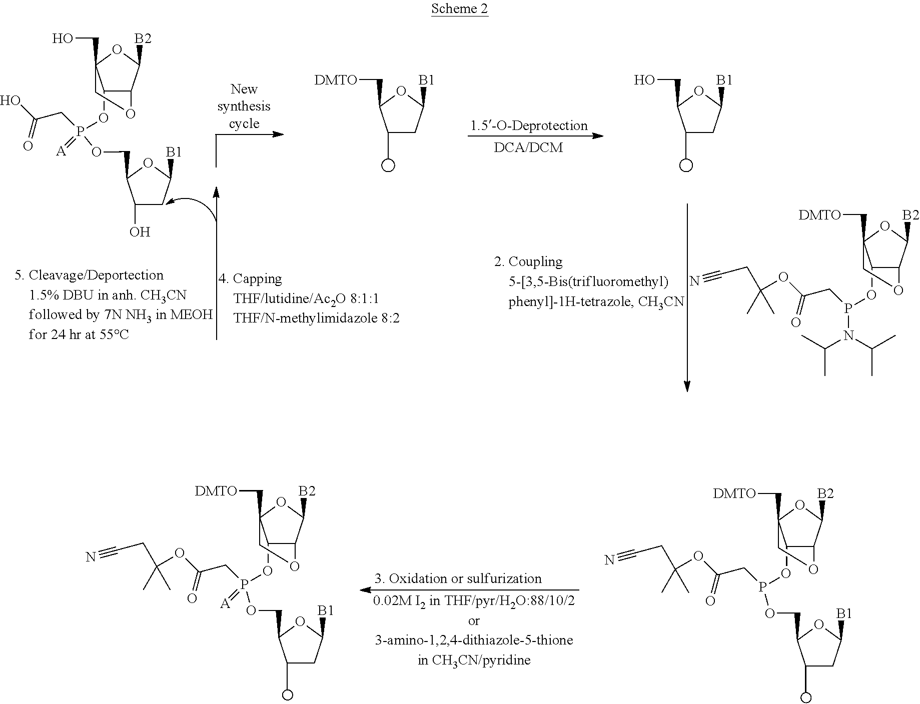

In scheme 2, B1 and B2 are nucleobases and A is as defined above.

The oligonucleotides comprising a phosphonoacetate or thiophosphonoacetate modification can be synthesized using solid phase oligonucleotide chemistry. DMT protected deoxyribonucleoside 3′-O-diisopropylaminophosphinoacetic acid dimethyl-β-cyanoethyl esters are condensed to a deoxyribonucleoside linked to the solid support. The phosphinite linkage is then oxidized using e.g., a low oxidizer reagent (0.02M I₂ in THF/pyridine/H₂O:88/10/2) or sulfurized using e.g., a 0.1M solution of 3-amino-1,2,4-dithiazole-5-thione in acetonitrile/pyridine. Following capping with acetic anhydride and treatment with dichloroacetic acid to remove the 5′-O-dimethoxytriyl group, the cycle is repeated an appropriate number of times to afford the oligonucleotide containing a phosphonoacetate modification.

The monomer building blocks useful in the manufacture of the oligonucleotide according to the invention can for example be prepared according to the following scheme.

Dimethylcyanoethylbromoacetate is synthesized by condensing bromoacetyl bromide with 3-hydroxy-3-methylbutyronitrile in toluene under reflux overnight. The phosphorous ester derivative is then prepared via a Reformatsky reaction with diisopropylamino chlorophosphine. Further condensation of this reactant with protected 2′-deoxynucleosides using tetrazole leads to the LNA PACE phosphoramidites.

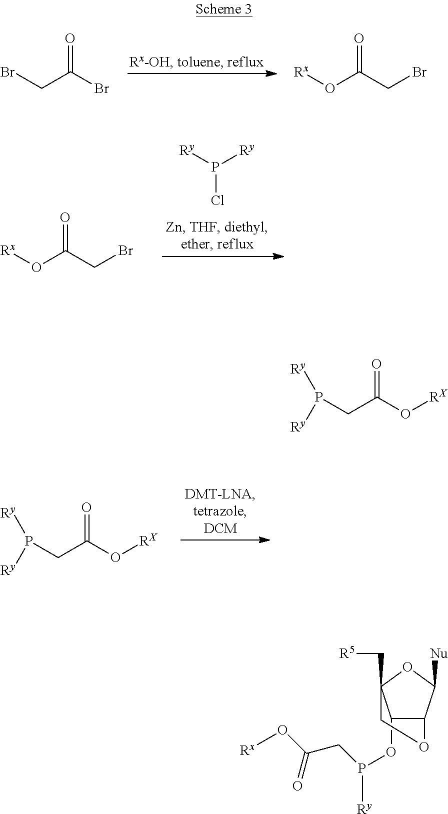

In scheme 3, R⁵, R^{x}, R^{y} and Nu are as defined above.

A monomer can in particular be prepared according to the following scheme following the above procedure.

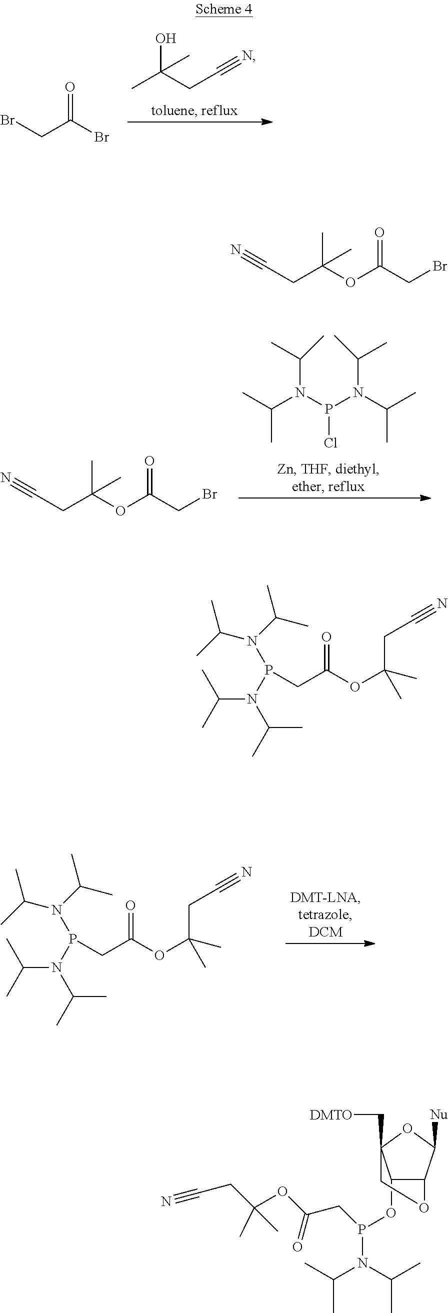

In scheme 4, Nu is as defined above.

The invention thus also relates to a compound of formula (II)

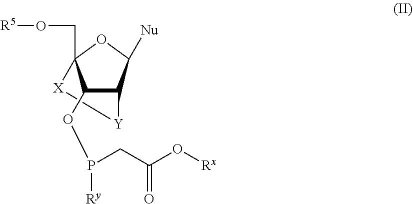

wherein
X is oxygen, sulfur, —CR^{a}R^{b}—, —C(R^{a})═C(R^{b})—, —C(═CR^{a}R^{b})—, —C(R^{a})═N—, —Si(R^{a})₂—, —SO₂—, —NR^{a}—; —O—NR^{a}—, —NR^{a}—O—, —C(=J)-, Se, —O—NR^{a}—, —NR^{a}—CR^{a}R^{b}—, —N(R^{a})—O— or —O—CR^{a}R^{b}—;
Y is oxygen, sulfur, —(CR^{a}R^{b})ₙ—, —CR^{a}R^{b}—O—CR^{a}R^{b}—, —C(R^{a})═C(R^{b})—, —C(R^{a})═N—, —Si(R^{a})₂—, —SO₂—, —NR^{a}—, —C(=J)-, Se, —O—NR^{a}—, —NR^{a}—CR^{a}R^{b}—, —N(R^{a})—O— or —O—CR^{a}R^{b}—;
with the proviso that —X—Y— is not —O—O—, Si(R^{a})₂—Si(R^{a})₂—, —SO₂—SO₂—, —C(R^{a})═C(R^{b})—C(R^{a})═C(R^{b}), —C(R^{a})═N—C(R^{a})═N—, —C(R^{a})═N—C(R^{a})═C(R^{b}), —C(R^{a})═C(R^{b})—C(R^{a})═N— or —Se—Se—;
J is oxygen, sulfur, ═CH₂ or ═N(R^{a});
R^{a} and R^{b} are independently selected from hydrogen, halogen, hydroxyl, cyano, thiohydroxyl, alkyl, substituted alkyl, alkenyl, substituted alkenyl, alkynyl, substituted alkynyl, alkoxy, substituted alkoxy, alkoxyalkyl, alkenyloxy, carboxyl, alkoxycarbonyl, alkylcarbonyl, formyl, aryl, heterocyclyl, amino, alkylamino, carbamoyl, alkylaminocarbonyl, aminoalkylaminocarbonyl, alkylaminoalkylaminocarbonyl, alkylcarbonylamino, carbamido, alkanoyloxy, sulfonyl, alkylsulfonyloxy, nitro, azido, thiohydroxylsulfidealkylsulfanyl, aryloxycarbonyl, aryloxy, arylcarbonyl, heteroaryl, heteroaryloxycarbonyl, heteroaryloxy, heteroarylcarbonyl, —OC(═X^{a})R^{c}, —OC(═X^{a})NR^{c}R^{d} and —NR^{e}C(═X^{a})NR^{c}R^{d};
or two geminal R^{a} and R^{b} together form optionally substituted methylene;
or two geminal R^{a} and R^{b}, together with the carbon atom to which they are attached, form cycloalkyl or halocycloalkyl, with only one carbon atom of —X—Y—;
wherein substituted alkyl, substituted alkenyl, substituted alkynyl, substituted alkoxy and substituted methylene are alkyl, alkenyl, alkynyl and methylene substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl, formyl, heterocylyl, aryl and heteroaryl;
X^{a} is oxygen, sulfur or —NR^{c};
R^{c}, R^{d} and R^{e} are independently selected from hydrogen and alkyl;
R⁵ is a hydroxyl protecting group;
R^{x} is cyanoalkyl or alkyl, in particular cyanoalkyl;
R^{y} is dialkylamino or pyrrolidinyl; and
Nu is a nucleobase or a protected nucleobase; and
n is 1, 2 or 3;
or a pharmaceutically acceptable alt thereof.

The invention further relates in particular to:

A compound according to the invention wherein —X—Y— is —CH₂—O—, —CH(CH₃)—O— or —CH₂CH₂—O—;

A compound according to the invention of formula (III) or (IV)

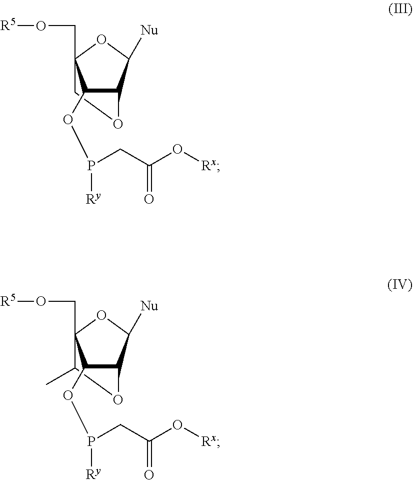

wherein R⁵, R^{x}, R^{y} and Nu are as defined above;

A compound according to the invention wherein R^{x} is 2-cyano-1,1-dimethyl-ethyl, methyl, ethyl, propyl or tert-butyl;

A compound according to the invention wherein R^{x} is 2-cyano-1,1-dimethyl-ethyl;

A compound according to the invention wherein R^{y} is diisopropylamino or pyrrolidinyl;

A compound according to the invention wherein R^{y} is dialkylamino;

A compound according to any one of claims 1 to 6, wherein R^{y} is diisopropylamino;

A compound according to the invention of formula (V):

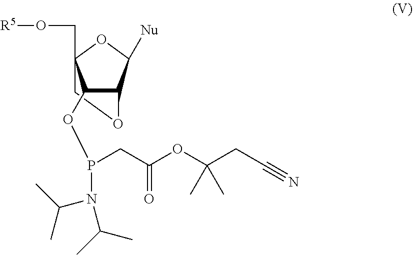

wherein R⁵ and Nu are as defined above;

A compound according to the invention wherein Nu is thymine, protected thymine, adenosine, protected adenosine, cytosine, protected cytosine, 5-methylcytosine, protected 5-methylcytosine, guanine, protected guanine, uracyl or protected uracyl.

A compound according to the invention selected from

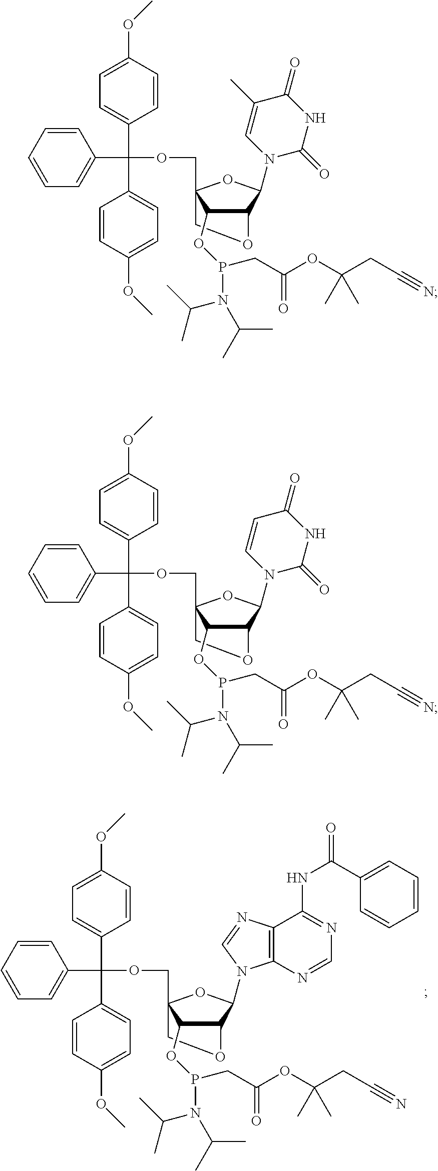 [SMILES: COc1ccc(C(OC[C@]23CO[C@@H](C2OP(CC(=O)OC(C)(C)CC#N)N(C(C)C)C(C)C)[C@H](n2cc(C)c(=O)[nH]c2=O)O3)(c2ccccc2)c2ccc(OC)cc2)cc1 ; COc1ccc(C(OC[C@]23CO[C@@H](C2OP(CC(=O)OC(C)(C)CC#N)N(C(C)C)C(C)C)[C@H](n2ccc(=O)[nH]c2=O)O3)(c2ccccc2)c2ccc(OC)cc2)cc1 ; COc1ccc(C(OC[C@]23CO[C@@H](C2OP(CC(=O)OC(C)(C)CC#N)N(C(C)C)C(C)C)[C@H](n2cnc4c(NC(=O)c5ccccc5)ncnc42)O3)(c2ccccc2)c2ccc(OC)cc2)cc1]

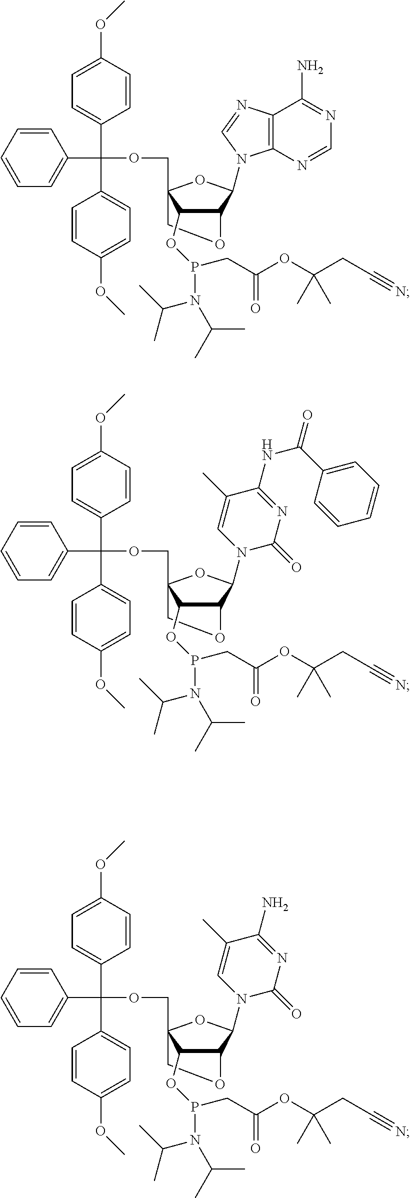 [SMILES: COc1ccc(C(OC[C@]23CO[C@@H](C2OP(CC(=O)OC(C)(C)CC#N)N(C(C)C)C(C)C)[C@H](n2cnc4c(N)ncnc42)O3)(c2ccccc2)c2ccc(OC)cc2)cc1 ; COc1ccc(C(OC[C@]23CO[C@@H](C2OP(CC(=O)OC(C)(C)CC#N)N(C(C)C)C(C)C)[C@H](n2cc(C)c(NC(=O)c4ccccc4)nc2=O)O3)(c2ccccc2)c2ccc(OC)cc2)cc1 ; COc1ccc(C(OC[C@]23CO[C@@H](C2OP(CC(=O)OC(C)(C)CC#N)N(C(C)C)C(C)C)[C@H](n2cc(C)c(N)nc2=O)O3)(c2ccccc2)c2ccc(OC)cc2)cc1]

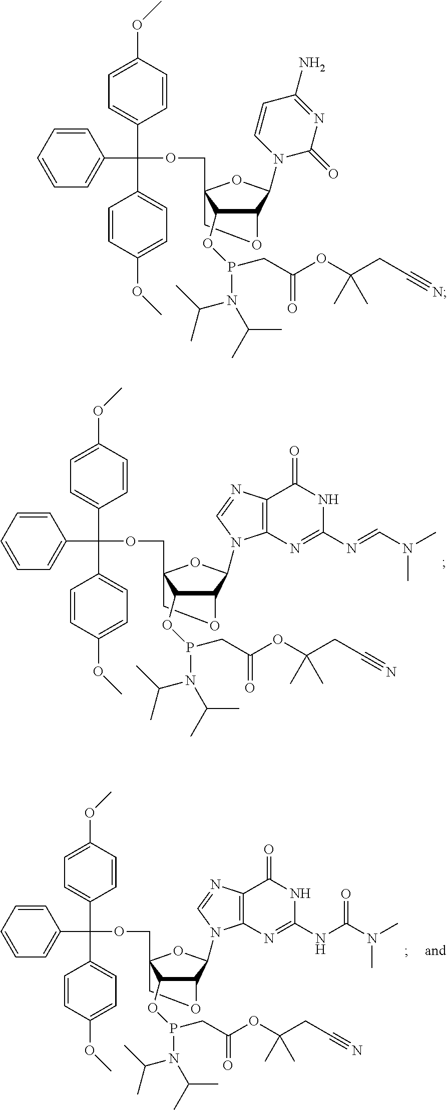 [SMILES: COc1ccc(C(OC[C@]23CO[C@@H](C2OP(CC(=O)OC(C)(C)CC#N)N(C(C)C)C(C)C)[C@H](n2ccc(N)nc2=O)O3)(c2ccccc2)c2ccc(OC)cc2)cc1 ; COc1ccc(C(OC[C@]23CO[C@@H](C2OP(CC(=O)OC(C)(C)CC#N)N(C(C)C)C(C)C)[C@H](n2cnc4c(=O)[nH]c(/N=C/N(C)C)nc42)O3)(c2ccccc2)c2ccc(OC)cc2)cc1 ; COc1ccc(C(OC[C@]23CO[C@@H](C2OP(CC(=O)OC(C)(C)CC#N)N(C(C)C)C(C)C)[C@H](n2cnc4c(=O)[nH]c(NC(=O)N(C)C)nc42)O3)(c2ccccc2)c2ccc(OC)cc2)cc1]

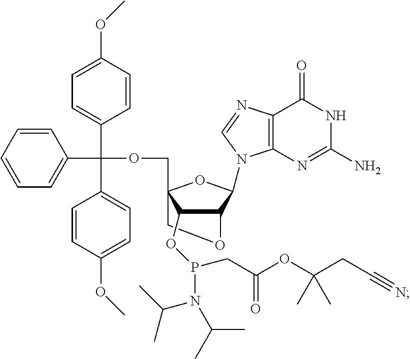 [SMILES: COc1ccc(C(OC[C@]23CO[C@@H](C2OP(CC(=O)OC(C)(C)CC#N)N(C(C)C)C(C)C)[C@H](n2cnc4c(=O)[nH]c(N)nc42)O3)(c2ccccc2)c2ccc(OC)cc2)cc1]

A process for the manufacture of a compound of formula (II) according to the invention comprising the reaction of a compound of formula (C)

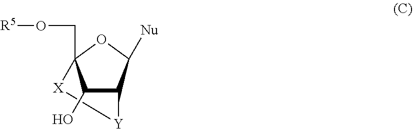

with a compound of formula P(R^{y})₂(CH₂)COO(R^{x}) in the presence of a coupling agent and base, wherein X, Y, R⁵, Nu, R^{x} and R^{y} are as defined above;

A process according to the invention wherein the coupling agent is 1H-tetrazole, 5-ethylthio-1H-tetrazole, 2-benzylthiotetrazole or 4,5-dicyanoimidazole (DCI), in particular tetrazole; and

The use of a compound according to the invention in the manufacture of an oligonucleotide.

The process of the invention can conveniently be quenched with a base, for example with triethylamine, pyridine, diisopropylamine or N,N-Diisopropylethylamine.

Oligonucleotides comprising a 2′-alkoxy-RNA, in particular 2′-methoxy-RNA, 2′-alkoxyalkoxy-RNA, in particular 2′-methoxyethoxy-RNA, according to the invention can be synthesized according to the following procedure.

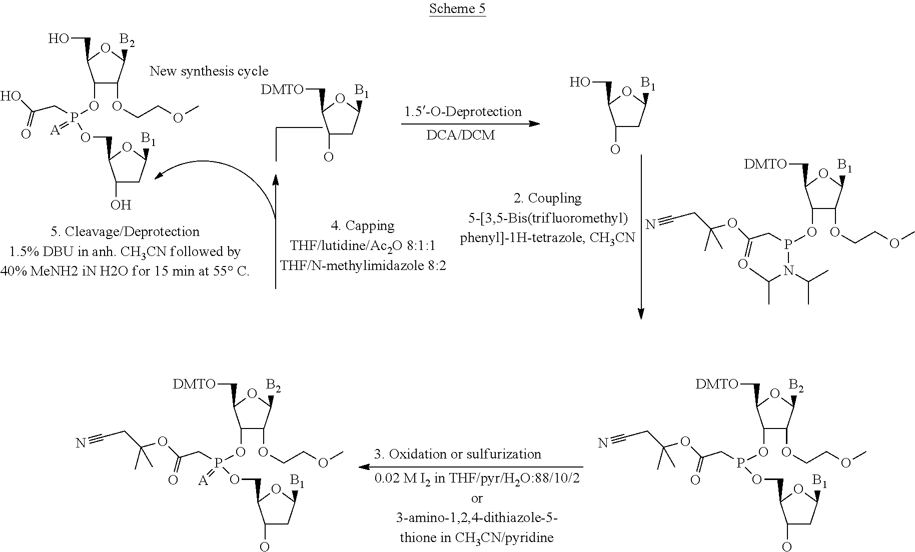

In scheme 5, B1 and B2 are nucleobases and A is as defined above. The oligonucleotides comprising a MOE (or other 2′ substituents) phosphonoacetate or thiophosphonoacetate modification can be synthesized using solid phase oligonucleotide chemistry. DMT protected deoxyribonucleoside 3′-O-diisopropylaminophosphinoacetic acid dimethyl-β-cyanoethyl esters are condensed to a deoxyribonucleoside linked to the solid support. The phosphinite linkage is then oxidized using e.g., a low oxidizer reagent (0.02M I₂ in THF/pyridine/H₂O:88/10/2) or sulfurized using e.g., a 0.1M solution of 3-amino-1,2,4-dithiazole-5-thione in acetonitrile/pyridine. Following capping with acetic anhydride and treatment with dichloroacetic acid to remove the 5′-O-dimethoxytriyl group, the cycle is repeated an appropriate number of times to afford the oligonucleotide containing a phosphonoacetate modification.

The monomer building blocks useful in the manufacture of the oligonucleotide according to the invention can for example be prepared according to the following scheme.

Dimethylcyanoethylbromoacetate is synthesized by condensing bromoacetyl bromide with 3-hydroxy-3-methylbutyronitrile in toluene under reflux overnight. The phosphorous ester derivative is then prepared via a Reformatsky reaction with diisopropylamino chlorophosphine. Further condensation of this reactant with protected 2′-deoxynucleosides using 4,5-DCI leads to the MOE PACE phosphoramidites.

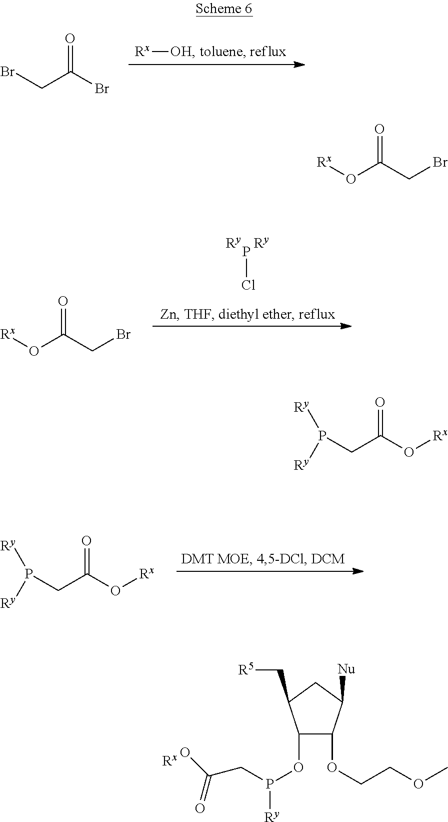

In scheme 6, R⁵, R^{x}, R^{y} and Nu are as defined above.

A monomer can in particular be prepared according to the following scheme following the above procedure.

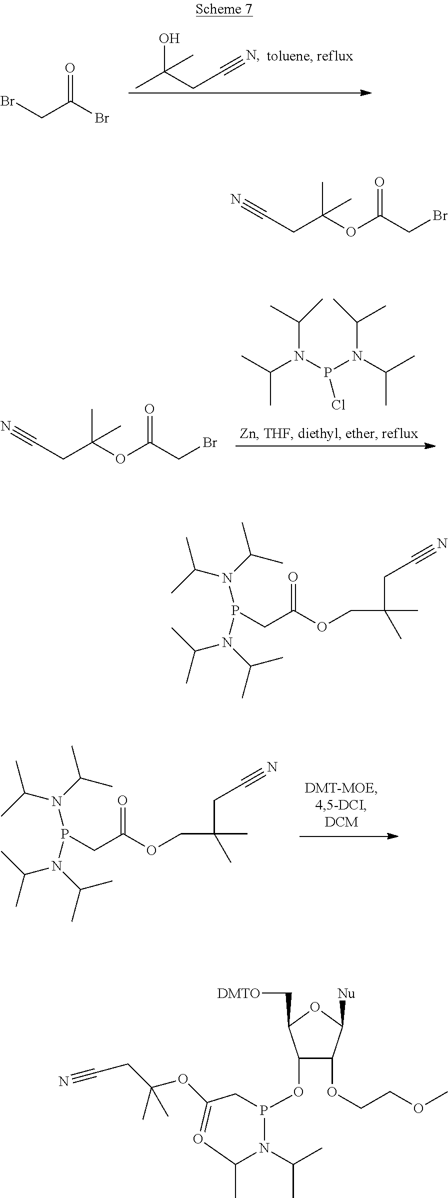

In scheme 7, Nu is as defined above.

The invention thus also relates to a compound of formula (VI)

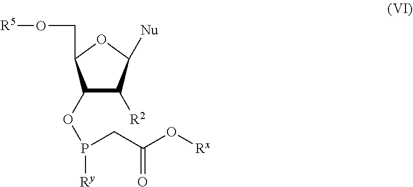

wherein

R² is alkoxy, alkoxyalkoxy or amino, in particular alkoxy or alkoxyalkoxy;

R⁵ is a hydroxyl protecting group;

R^{x} is cyanoalkyl or alkyl, in particular cyanoalkyl;

R^{y} is dialkylamino or pyrrolidinyl; and

Nu is a nucleobase or a protected nucleobase; and

or a pharmaceutically acceptable alt thereof.

The invention further relates in particular to:

A compound according to the invention wherein R² is methoxy, methoxyethoxy or amino, in particular methoxy or methoxyethoxy;

A compound according to the invention of formula (VII)

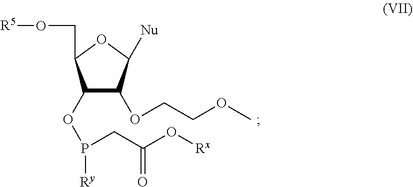

wherein R⁵, R^{x}, R^{y} and Nu are as defined above;

A compound according to the invention wherein R^{x} is 2-cyano-1,1-dimethyl-ethyl, methyl, ethyl, propyl or tert-butyl;

A compound according to the invention wherein R^{x} is 2-cyano-1,1-dimethyl-ethyl;

A compound according to the invention wherein R^{y} is diisopropylamino or pyrrolidinyl;

A compound according to the invention wherein R^{y} is dialkylamino;

A compound according to any one of claims 1 to 6, wherein R^{y} is diisopropylamino;

A compound according to the invention of formula (VIII)

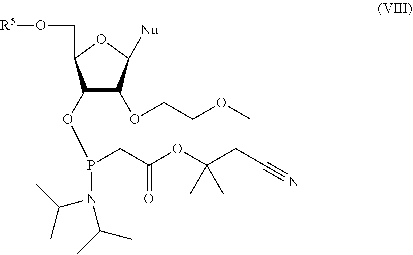

wherein R⁵ and Nu are as defined above;

A compound according to the invention wherein Nu is thymine, protected thymine, adenosine, protected adenosine, cytosine, protected cytosine, 5-methylcytosine, protected 5-methylcytosine, guanine, protected guanine, uracyl or protected uracyl.

A compound according to the invention selected from

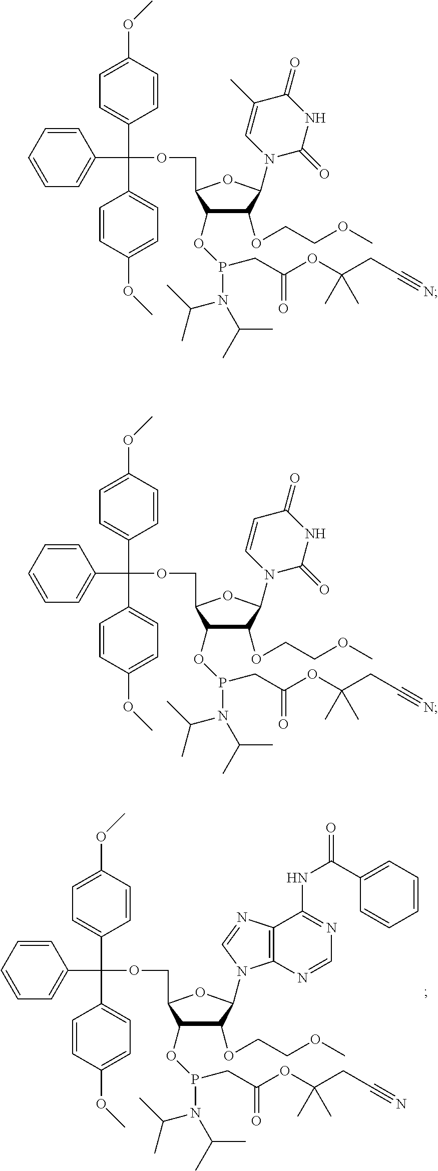

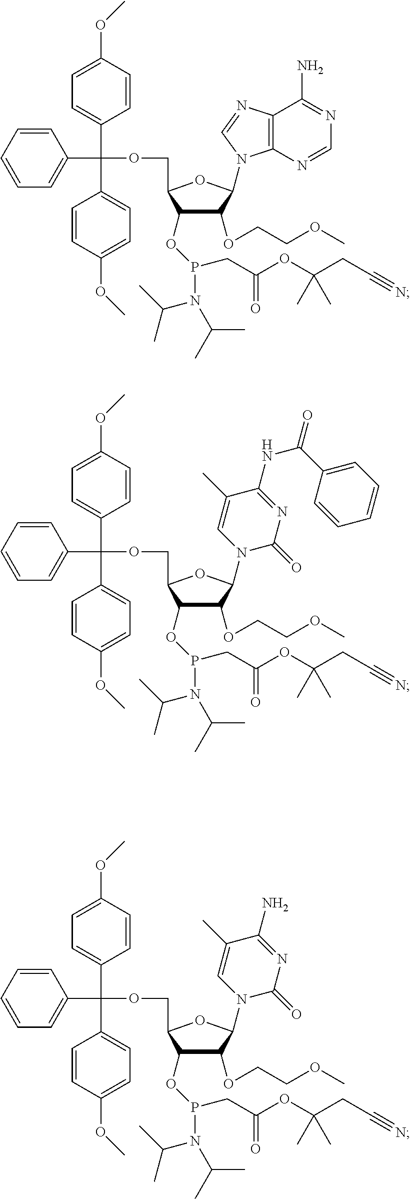

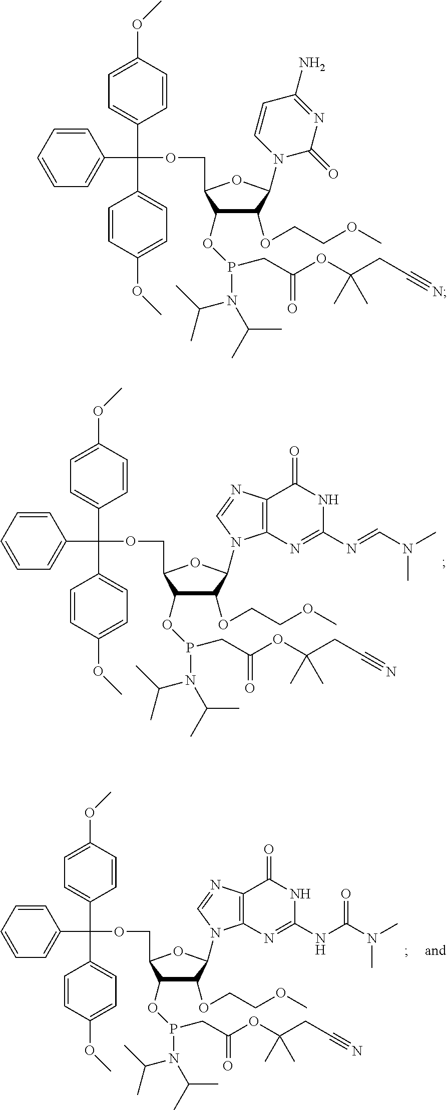

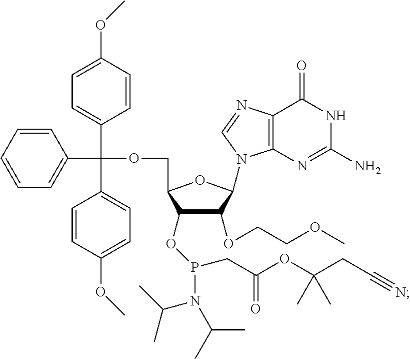

A process for the manufacture of a compound of formula (VI) according to the invention comprising the reaction of a compound of formula (D)

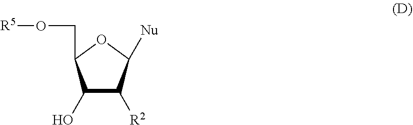

with a compound of formula P(R^{y})₂(CH₂)COO(R^{x}) in the presence of a coupling agent and base, wherein R², R⁵, Nu, R^{x} and R^{y} are as defined above;

A process according to the invention wherein the coupling agent is 1H-tetrazole, 5-ethylthio-1H-tetrazole, 2-benzylthiotetrazole, 4,5-dicyanoimidazole (DCI), in particular DCI; and

The use of a compound according to the invention in the manufacture of an oligonucleotide.

The process of the invention can conveniently be quenched with a base, for example with triethylamine, pyridine, diisopropylamine or N,N-Diisopropylethylamine.

The invention will now be illustrated by the following examples which have no limiting character.

### EXAMPLES

Abbreviations:

### A Adenine

### G Guanine

ₘC methyl Cytosine

### T Thymine

### LNA Locked Nucleic Acid

### RNA Ribonucleic Acid

### DMT Dimetoxytrityl

DCA Dichloroacetic acid

### DCM Dichloromethane

### THF Tetrahydrofuran

Anh. Anhydrous

### TLC Thin-layer Chromatography

### NMR Nuclear Magnetic Resonance

### CPG Controlled Pore Glass

### RT Reverse Transcription

qPCR quantitative Polymerase Chain reaction
ds double stranded
Tm Thermal melting

### Example 1: Monomer Synthesis

### 1.1. 1-cyano-2-methylpropan-2-yl 2-bromoacetate

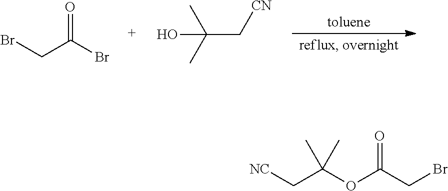

To a solution of 2-bromoacetyl bromide (14.7 g, 6.31 mL, 72.6 mmol, 1.2 eq) in toluene (67.2 mL), 3-hydroxy-3-methylbutanenitrile (6 g, 6.28 ml, 60.5 mmol, 1 eq) was slowly added while stirring. The round-bottom flask was fitted with a Friedrich's condenser and a drying tube vented to an acid trap (containing NaOH aq.). The reaction mixture was heated to reflux overnight. The reaction was allowed to cool down to room temperature and the mixture was then concentrated in vacuo to result in a colourless oil. The crude was purified by Combiflash Chromatography using ethyl acetate/hexane as gradients, the product was eluted at 30% ethyl acetate in hexane to afford 1-cyano-2-methylpropan-2-yl 2-(bis(diisopropylamino)phosphaneyl)acetate (8.14 g, 37 mmol, 58% yield). ¹H NMR (CHLOROFORM-d, 300 MHz) δ 3.8 (s, 2H), 2.9 (s, 2H), 1.6 (s, 6H).

### 1.2. 1-cyano-2-methylpropan-2-yl 2-(bis(diisopropylamino)phosphaneyl)acetate

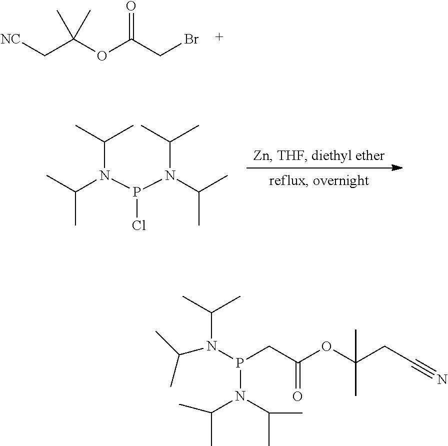

1-chloro-N,N,N′,N′-tetraisopropylphosphanediamine (7.75 g, 29 mmol, 1 eq) was dissolved in anhydrous THF (69.4 ml). Another 41.6 ml of anh. diethyl ether were added, 1-cyano-2-methylpropan-2-yl 2-bromoacetate (7.03 g, 32 mmol, 1.1 eq) in anh. THF (34.7 ml) was placed in a round bottom flask. Zinc (2.85 g, 43.6 mmol, 1.5 eq), anh. diethyl ether (22.2 ml) and a magnetic stir bar were placed in a 500 mL three necked round-bottom flask fitted with a Friedrich's condenser. The phosphine (36 mL) and the bromoacetate solutions (10 mL) were added simultaneously and very slowly to the three necked round-bottom flask. The reaction mixture was then heated under reflux until an exothermic reaction was noticeable (the slightly cloudy, colorless reaction became clear and slightly yellow). The reaction was continued at reflux by the addition of the remainder of the phosphine and bromoacetate solutions. Once the addition was complete, the reaction was kept at reflux for 45 min by heating, allowed to cool down to room temperature and analyzed for completeness by ³¹P NMR. The starting material at δ=135 ppm was converted to a single product at δ=48 ppm. The cooled reaction mixture was concentrated in vacuo to afford a viscous oil. The resulting material was dissolved with anhydrous heptane and a small amount of acetonitrile to fully dissolve the crude product. This solution was extracted twice with anh. heptane. The acetonitrile layer was analyzed by ³¹P NMR for absence of the product at δ=48 ppm and discarded. All heptane fractions were combined and concentrated in vacuo to give a slightly yellow oil. It was then dried under high vacuum overnight resulting in a white solid (7.096 g, 19 mmol, 62% yield). ¹H NMR (CHLOROFORM-d, 300 MHz) δ 3.3-3.5 (m, 4H), 2.9 (s, 2H), 2.7 (d, 2H), 1.60 (s, 6H), 1.3 (m, 24H).

### 1.3. (1-cyano-2-methylpropan-2-yl) 2-[[di(propan-2-yl)amino]-[[rac-(1R,3R)-1-[[bis(4-methoxyphenyl)-phenylmethoxy]methyl]-3-(5-methyl-2,4-dioxopyrimidin-1-yl)-2,5-dioxabicyclo[2.2.1]heptan-7-yl]oxy]phosphanyl]acetate

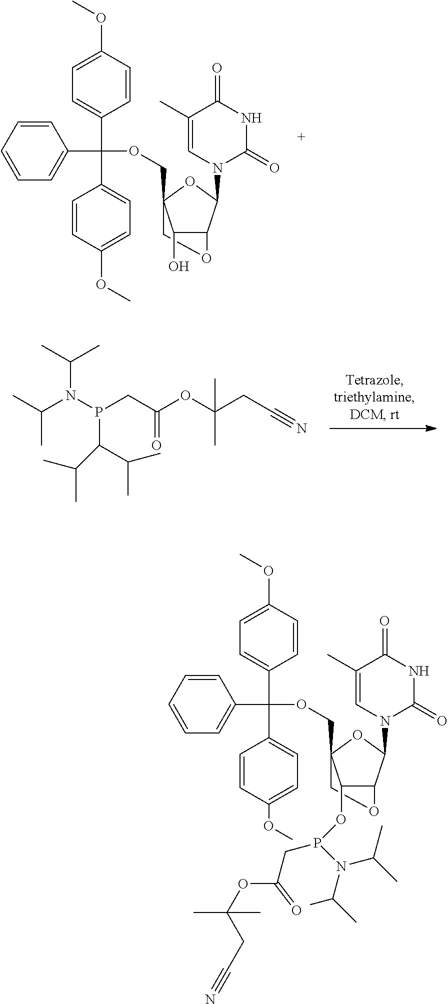 [SMILES: COc1ccc(C(OC[C@]23COC(C2O)[C@H](n2cc(C)c(=O)[nH]c2=O)O3)(c2ccccc2)c2ccc(OC)cc2)cc1 ; CC(C)C(C(C)C)P(CC(=O)OC(C)(C)CC#N)N(C(C)C)C(C)C ; COc1ccc(C(OC[C@]23COC(C2OP(CC(=O)OC(C)(C)CC#N)N(C(C)C)C(C)C)[C@H](n2cc(C)c(=O)[nH]c2=O)O3)(c2ccccc2)c2ccc(OC)cc2)cc1]

1-[(1R,4R,6R,7S)-4-[[bis(4-methoxyphenyl)-phenyl-methoxy]methyl]-7-hydroxy-2,5-dioxabicyclo[2.2.1]heptan-6-yl]-5-methyl-pyrimidine-2,4-dione (0.7 g, 1.22 mmol, 1 eq) was dissolved in anh. DCM (15.3 ml), 1-cyano-2-methylpropan-2-yl 2-(bis(diisopropylamino)phosphaneyl)acetate (545 mg, 1.47 mmol, 1.2 eq) was then added to the reaction mixture. Upon complete dissolution of the reaction components, tetrazole (2.17 ml, 978 μmol, 0.8 eq) was added to the reaction mixture as a 0.45 M solution in anh. CH₃CN. The reaction mixture was then allowed to stir at room temperature overnight under argon and analyzed by ³¹P NMR and silica gel TLC (eluted with ethyl acetate). The reaction was determined to be complete by spot to spot conversion to a faster eluting product on TLC and by a complete loss of the acetic acid phosphinodiamite ³¹P NMR signal. Upon completion, the reaction was quenched by the addition of triethylamine (99 mg, 136 μl, 978 μmol, 0.8 eq). After 5 min, the reaction mixture was concentrated in vacuo to afford a viscous colourless oil. The product was redissolved in a minimum volume of ethyl acetate and purified via a column chromatography (80/20: ethyl acetate/heptane). The fractions containing the product were combined and concentrated, resulting in a foam which was redissolved in a minimal amount of anh. DCM. Heptane was added dropwise to rapidly stirring. The solid precipitate was isolated by filtration and dried overnight in vacuo to afford 743 mg of target compound as a white solid (743 mg, 0.88 mmol, 69% yield). ³¹P NMR (CHLOROFORM-d, 121 MHz) δ 126.91 (s, IP), 122.25 (s, IP). ¹H NMR (600 MHz, ACETONITRILE-73) 5 ppm 8.89-9.22 (m, 1H), 7.57-7.59 (m, 1H), 7.50 (d, J=7.6 Hz, 1H), 7.33-7.39 (m, 3H), 7.33-7.37 (m, 2H), 7.26-7.31 (m, 1H), 6.88-6.95 (m, 4H), 5.58 (s, 1H), 4.62 (s, 1H), 4.14 (dJ,=6.8 Hz, 1H), 3.79-3.81 (m, 5H), 3.79-3.85 (m, 2H), 3.47-3.50 (m, 2H), 3.42-3.50 (m, 1H), 2.92-2.95 (m, 1H), 2.67-2.71 (m, 1H), 2.61-2.66 (m, 1H), 1.72 (s, 2H), 1.52 (d, J=5.2 Hz, 4H), 1.09 (d, J=6.7 Hz, 4H), 1.01 (br d, J=6.7 Hz, 4H). LCMS (ES+) found: 843.37 g/mol.

### 1.4. (1-cyano-2-methylpropan-2-yl) 2-[[di(propan-2-yl)amino]-[[rac-(1R,3R)-3-(6-benzamidopurin-9-yl)-1-[[bis(4-methoxyphenyl)-phenylmethoxy]methyl]-2,5-dioxabicyclo[2.2.1]heptan-7-yl]oxy]phosphanyl]acetate

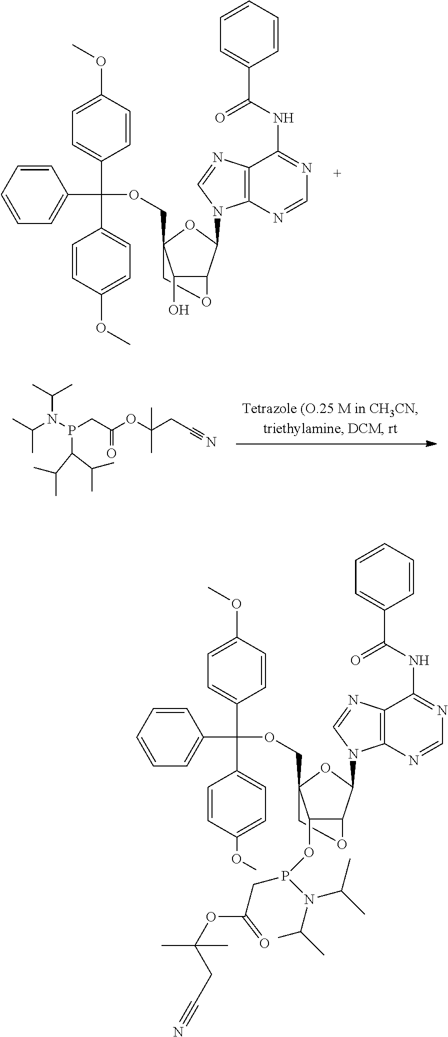 [SMILES: COc1ccc(C(OC[C@]23COC(C2O)[C@H](n2cnc4c(NC(=O)c5ccccc5)ncnc42)O3)(c2ccccc2)c2ccc(OC)cc2)cc1 ; CC(C)C(C(C)C)P(CC(=O)OC(C)(C)CC#N)N(C(C)C)C(C)C ; COc1ccc(C(OC[C@]23COC(C2OP(CC(=O)OC(C)(C)CC#N)N(C(C)C)C(C)C)[C@H](n2cnc4c(NC(=O)c5ccccc5)ncnc42)O3)(c2ccccc2)c2ccc(OC)cc2)cc1]

N-[9-[(1R,4R,6R,7S)-4-[[bis(4-methoxyphenyl)-phenyl-methoxy]methyl]-7-hydroxy-2,5-dioxabicyclo[2.2.1]heptan-6-yl]purin-6-yl]benzamide (3 g, 4.37 mmol, 1 eq) was dissolved in anh. DCM (54.7 ml), 1-cyano-2-methylpropan-2-yl 2-(bis(diisopropylamino)phosphaneyl)acetate (1.95 g, 5.25 mmol, 1.2 eq) was then added to the reaction mixture. Upon complete dissolution of the reaction components, tetrazole (7.78 ml, 3.5 mmol, 0.8 eq) was added to the reaction mixture as a 0.45 M solution in anh. CH₃CN. The reaction mixture was allowed to stir at room temperature overnight under argon and analyzed by ³¹P NMR and silica gel TLC (eluted with ethyl acetate). The reaction was determined to be complete by spot to spot conversion to a faster eluting product on TLC and by a complete loss of the acetic acid phosphinodiamite ³¹P NMR signal. Upon completion, the reaction was quenched by the addition of triethylamine (354 mg, 488 μl, 3.5 mmol, 0.8 eq). After 5 min, the reaction mixture was concentrated in vacuo to afford a viscous colourless oil. The product was redissolved in a minimum volume of ethyl acetate and purified via a column chromatography (80/20: ethyl acetate/heptane). The fractions containing the product were combined and concentrated, resulting in a foam which was redissolved in a minimal amount of anh. DCM. Heptane was added dropwise to rapidly stirring. The solid precipitate was isolated by filtration and dried overnight in vacuo to afford 1.86 g of target compound as a white solid (1.86 g, 1.9 mmol, 45% yield). ³¹P NMR (ACETONITRILE-d₃, 121 MHz) δ 125.2 (s, IP), 120.9 (s, IP). LCMS (ES+) found: 956.40 g/mol.

### 1.5. (1-cyano-2-methylpropan-2-yl) 2-[[di(propan-2-yl)amino]-[[rac-(1R,3R)-3-(4-benzamido-5-methyl-2-oxopyrimidin-1-yl)-1-[[bis(4-methoxyphenyl)-phenylmethoxy]methyl]-2,5-dioxabicyclo[2.2.1]heptan-7-yl]oxy]phosphanyl]acetate

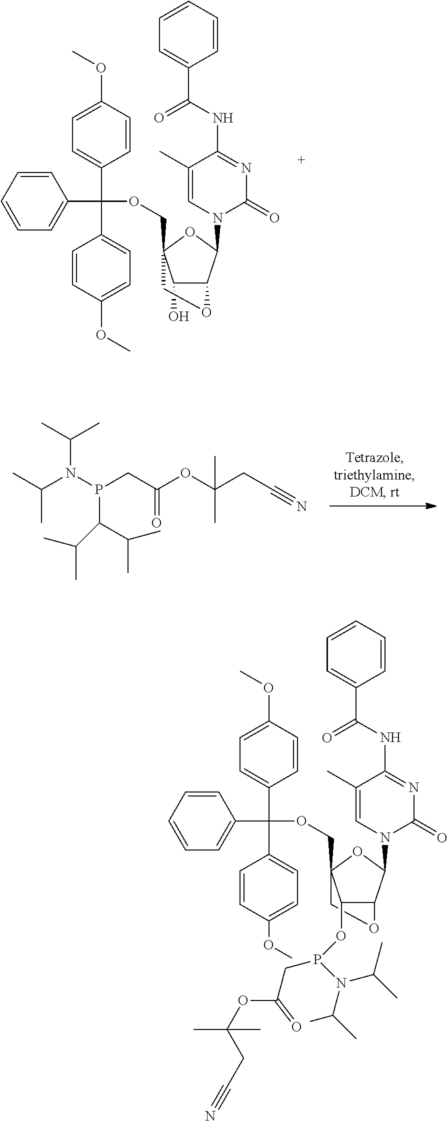 [SMILES: COc1ccc(C(OC[C@@]23CO[C@@H]([C@H](n4cc(C)c(NC(=O)c5ccccc5)nc4=O)O2)[C@@H]3O)(c2ccccc2)c2ccc(OC)cc2)cc1 ; CC(C)C(C(C)C)P(CC(=O)OC(C)(C)CC#N)N(C(C)C)C(C)C ; COc1ccc(C(OC[C@]23COC(C2OP(CC(=O)OC(C)(C)CC#N)N(C(C)C)C(C)C)[C@H](n2cc(C)c(NC(=O)c4ccccc4)nc2=O)O3)(c2ccccc2)c2ccc(OC)cc2)cc1]

N-[1-[(1R,4R,6R,7S)-4-[[bis(4-methoxyphenyl)-phenyl-methoxy]methyl]-7-hydroxy-2,5-dioxabicyclo[2.2.1]heptan-6-yl]-5-methyl-2-oxo-pyrimidin-4-yl]benzamide (2.8 g, 4.14 mmol, 1 eq) was dissolved in anh. DCM (59.2 ml), 1-cyano-2-methylpropan-2-yl 2-(bis(diisopropylamino)phosphaneyl)acetate (1.85 g, 4.97 mmol, 1.2 eq) was then added to the reaction mixture. Upon complete dissolution of the reaction components, tetrazole (7.37 ml, 3.31 mmol, 0.8 eq) was added to the reaction mixture as a 0.45 M solution in anh. CH₃CN. The reaction mixture was allowed to stir at room temperature overnight under argon and analyzed by ³¹P NMR and silica gel TLC (eluted with ethyl acetate). The reaction was determined to be complete by spot to spot conversion to a faster eluting product on TLC and by a complete loss of the acetic acid phosphinodiamite ³¹P NMR signal. Upon completion, the reaction was quenched by the addition of triethylamine (335 mg, 462 μl, 3.31 mmol, 0.8 eq). After 5 min, the reaction mixture was concentrated in vacuo to afford a viscous slightly yellow oil. The product was redissolved in a minimum volume of ethyl acetate and purified via a column chromatography (50/50: ethyl acetate/heptane). The fractions containing the product were combined and concentrated, resulting in a foam which was redissolved in a minimal amount of anh. DCM. Heptane was added dropwise to rapidly stirring. The solid precipitate was isolated by filtration and dried overnight in vacuo to afford 2.35 g of target compound as a light yellow solid (2.35 g, 2.22 mmol, 46% yield). ³¹P NMR (ACETONITRILE-d₃, 121 MHz) δ 126.78 (s, IP), 122.73 (s, IP). LCMS (ES+) found: 947.41 g/mol.

### 1.6. (1-cyano-2-methylpropan-2-yl) 2-[[di(propan-2-yl)amino]-[[rac-(1R,3R)-1-[[bis(4-methoxyphenyl)-phenylmethoxy]methyl]-3-[2-(2-methylpropanoylamino)-6-oxo-1H-purin-9-yl]-2,5-dioxabicyclo[2.2.1]heptan-7-yl]oxy]phosphanyl]acetate

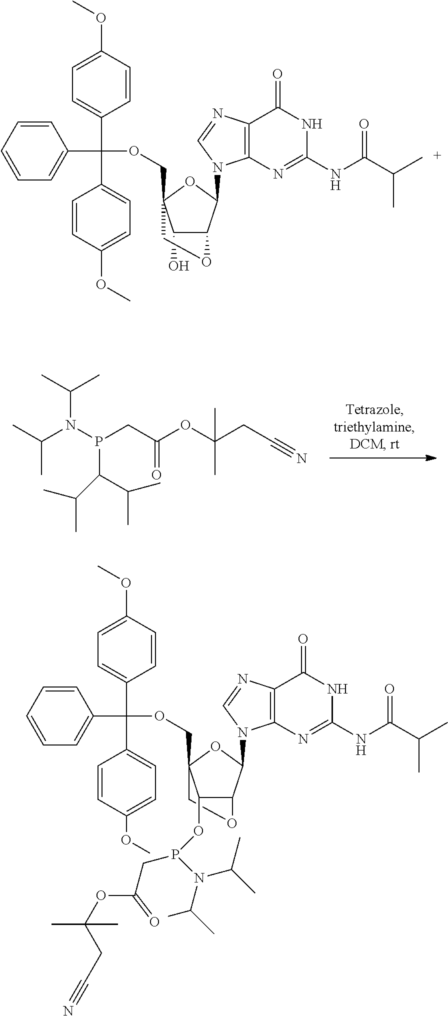

N′-[9-[(1R,4R,6R,7S)-4-[[bis(4-methoxyphenyl)-phenyl-methoxy]methyl]-7-hydroxy-2,5-dioxabicyclo [2.2.1]heptan-6-yl]-6-oxo-1H-purin-2-yl]-N,N-dimethyl-formamidine (2.6 g, 3.89 mmol, 1 eq) was dissolved in anh. DCM (55.6 ml), 1-cyano-2-methylpropan-2-yl 2-(bis(diisopropylamino)phosphaneyl)acetate (1.74 g, 4.67 mmol, 1.2 eq) was then added to the reaction mixture. Upon complete dissolution of the reaction components, tetrazole (6.92 ml, 3.12 mmol, Eq: 0.8) was added to the reaction mixture as a 0.45 M solution in anh. CH₃CN. The reaction mixture was allowed to stir at RT overnight under argon and analyzed by ³¹P NMR and silica gel TLC (eluted with ethyl acetate). The reaction was determined to be complete by spot to spot conversion to a faster eluting product on TLC and by a complete loss of the acetic acid phosphinodiamite ³¹P NMR signal. Upon completion, the reaction was quenched by the addition of triethylamine (315 mg, 434 μl, 3.12 mmol, 0.8 eq). After 5 min, the reaction mixture was concentrated in vacuo to afford a viscous colourless oil. The product was redissolved in a minimum volume of ethyl acetate and purified via a column chromatography (100% ethyl acetate). The fractions containing the product were combined and concentrated, resulting in a foam which was redissolved in a minimal amount of anh. DCM. Heptane was added dropwise to rapidly stirring. The solid precipitate was isolated by filtration and dried overnight in vacuo to afford 1.4 g of target compound as a white solid (1.4 g, 1.4 mmol, 38% yield). ³¹P NMR (ACETONITRILE-d₃, 121 MHz) δ 126.48 (s, IP), 121.3 (s, IP). LCMS (ES+) found: 938.42 g/mol.

### Example 2: Oligonucleotide Synthesis

Oligonucleotides were synthesized using a MerMade 12 automated DNA synthesizer by Bioautomation. Syntheses were conducted on a 1 μmol scale using a controlled pore glass support (500 Å) bearing a universal linker.

In standard cycle procedures for the coupling of standard DNA and LNA phosphoramidites DMT deprotection was performed with 3% (w/v) dichloroacetic acid in CH₂Cl₂ in three applications of 230 μL for 105 sec. The respective phosphoramidites were coupled three times with 95 μL of 0.1M solutions in acetonitrile (or acetonitrile/CH₂C₁₋₂ 1:1 for the LNA-^{Me}C building block) and 110 μL of a 0.25M solution of 5-[3,5-Bis(trifluoromethyl)phenyl]-2H-tetrazole as an activator and a coupling time of 180 sec. Sulfurization was performed using a 0.1M solution of 3-amino-1,2,4-dithiazole-5-thione in acetonitrile/pyridine in one application of 200 μL for 3 minutes. Oxidation was performed using a 0.02M I₂ in THF/pyr/H₂O:88/10/2 in one application for 3 minutes. Capping was performed using THF/lutidine/Ac₂O 8:1:1 (CapA, 75 μmol) and THF/N-methylimidazole 8:2 (CapB, 75 μmol) for 70 sec.

Synthesis cycles for the introduction of PACE LNAs included DMT deprotection using 3% (w/v) dichloroacetic acid in in CH₂Cl₂ in three applications of 230 μL for 105 sec. Freshly prepared LNA PACE were coupled two times with 95 μL of 0.1M solution in acetonitrile and 110 μL of a 0.25M solution of 5-[3,5-Bis(trifluoromethyl)phenyl]-277-tetrazole as an activator and a coupling time of 15 minutes. Sulfurization was performed using a 0.1M solution of 3-amino-1,2,4-dithiazole-5-thione in acetonitrile/pyridine in one application for 3 minutes. Oxidation was performed using a 0.02M I₂ in THF/pyr/H₂O:88/10/2 in one application for 3 minutes. Capping was performed using THF/lutidine/Ac₂O 8:1:1 (CapA, 75 μmol) and THF/N-methylimidazole 8:2 (CapB, 75 μmol) for 70 sec.

After the synthesis, a solution of 1.5% DBU in anh. CH₃CN was carefully passed through the column a few times to deprotect the dimethylcyanoethyl protecting groups and to prevent alkylation of the bases during deprotection. It was then allowed to stand at RT for 60 minutes. The solution was then discarded and the column was rinsed with 2-3 mL of anh. CH₃CN. It was then dried under stream of argon. The CPG was then transferred carefully into a 4 mL vial where 1 mL of 7N NH₃ in MeOH was added and left under stirring for 24 hr at 55° C.

Crude DMT-on oligonucleotides were purified by RP-HPLC purification using a C18 column followed by DMT removal with 80% aqueous acetic acid and ethanol precipitation or by cartridge purification. The PACE LNA phosphoramidites were synthesized in Basel. The normal phosphoramidites were ordered from Sigma Aldrich, as well as all of the reagents used in the solid phase synthesis.

The following molecules have been prepared following the above procedure.

| Compound | | Calculated | Found |
|---|---|---|---|
| ID No. | Sequence | mass | mass |
| #1 | G*^{m}CaagcatcctGT | 4295.5 | 4296.6 |
| #2 | G^{m}C*aagcatcctGT | 4295.5 | 4295.7 |
| #3 | G^{m}CaagcatcctG*T | 4295.5 | 4296.9 |
| #4 | G*^{m}C*aagcatcctGT | 4337.5 | 4340.1 |
| #5 | G*^{m}CaagcatcctG*T | 4337.5 | 4338.3 |
| #6 | G^{m}C*aagcatcctG*T | 4337.5 | 4338.3 |
| #7 | G*AGttacttgccaA^{m}CT | 5321.3 | 5322.3 |
| #8 | GA*GttacttgccaA^{m}CT | 5321.3 | 5321.7 |
| #9 | GAG*ttacttgccaA^{m}CT | 5321.3 | 5323.8 |
| #10 | GAGttacttgccaA*^{m}CT | 5321.3 | 5321.7 |
| #11 | GAGttacttgccaA^{m}C*T | 5321.3 | 5322.6 |
| #12 | G*AgttacttgccaA^{m}C*T | 5363.3 | 5364.3 |
| #13 | G^{m}CattggtatT*^{m}CA | 4367.6 | 4368.9 |
| #14 | G^{m}C*attggtatT^{m}CA | 4367.6 | 4368.9 |
| #15 | G^{m}CattggtatT^{m}C*A | 4367.6 | 4368.6 |
| #16 | G*^{m}CattggtatT^{m}CA | 4367.6 | 4368.0 |
| #17 | G*^{m}CattggtatT^{m}C*A | 4409.6 | 4409.7 |
| #18 | G^{m}C*attggtatT*^{m}CA | 4409.6 | 4409.4 |
| #19 | G^{m}C*attggtatT^{m}C*A | 4409.6 | 4409.4 |
| #20 | G*^{m}C*attggtatT^{m}CA | 4409.6 | 4408.5 |
| #21 | G^{m}CattggtatT*^{m}C*A | 4409.6 | 4409.4 |
| #22 | G*^{m}CattggtatT*^{m}CA | 4409.6 | 4410.3 |
| #23 | G*^{m}C*attggtatT*^{m}CA | 4451.6 | 4451.4 |

*PACE phosphorothioate modification between adjacent nucleotides
A, G, ^{m}C, T represent LNA nucleotides
a, g, c, t represent DNA nucleotides
all other linkages were prepared as phosphorothioates

### Example 3: In Vitro Efficacy of Oligonucleotides Targeting HIF1a mRNA in Human HeLa and A549 Cells at Different Concentrations for a Dose-Response Curve

HeLa and A549 cell lines were purchased from ATCC and maintained as recommended by the supplier in a humidified incubator at 37° C. with 5% CO₂. For assays, 3000 cells/well (HeLa) and 3500 cells/well (A549) were seeded in a 96 multi well plate in culture media. Cells were incubated for 24 hours before addition of oligonucleotides dissolved in PBS. Concentration range of oligonucleotides: highest concentration 25 μM, 1:1 dilutions in 8 steps. Three days after addition of oligonucleotides, the cells were harvested. RNA was extracted using the PureLink Pro 96 RNA Purification kit (Thermo Fisher Scientific) according to the manufacturer's instructions and eluted in 50 μl water. The RNA was subsequently diluted 10 times with DNase/RNase free Water (Gibco) and heated to 90° C. for one minute.

For gene expressions analysis, One Step RT-qPCR was performed using qScript™ XLT One-Step RT-qPCR ToughMix®, Low ROX™ (Quantabio) in a duplex set up. The following TaqMan primer assays were used for qPCR: HIF1A, Hs00936368_m1 with endogenous control GUSB, Hs99999908_m1 (VIC-MGB). All primer sets were purchased from Thermo Fisher Scientific. The relative expression level of HIF1A mRNA is shown as percent of control (PBS-treated cells) and IC₅₀ values have been determined using GraphPad Prism7 on data from n=2 biological replicates.

The results are shown in the tables below and in FIGS. 3 and 4.

| Compound | IC₅₀ in | | IC₅₀ in | |
|---|---|---|---|---|
| ID No. | HeLa (μM) | SD | A549 (μM) | SD |
| Control | 2.85 | 0.34 | 9.44 | 0.59 |
| #1 | 3.28 | 0.35 | 9.21 | 0.23 |
| #2 | 5.28 | 1.05 | 9.72 | 0.19 |
| #3 | 2.08 | 0.24 | 7.93 | 0.19 |
| #4 | 7.44 | 0.71 | 15.51 | 0.09 |
| #5 | 3.06 | 0.43 | 11.26 | 0.20 |
| #6 | 3.32 | 0.47 | 11.25 | 0.40 |

The data depicted in the plots of FIGS. 3 and 4 is reported in the tables below.

### HIF1A Expression in HeLa (Average of Biological Replicate)

| | #1 | #2 | #3 | #4 | #5 | #6 | Reference |
|---|---|---|---|---|---|---|---|
| 25.00 μM | 16 | 17 | 13 | 25 | 16 | 20 | 16 |
| 12.50 μM | 23 | 26 | 20 | 39 | 24 | 27 | 23 |
| 6.25 μM | 36 | 42 | 28 | 55 | 37 | 43 | 34 |
| 3.13 μM | 55 | 66 | 41 | 69 | 52 | 58 | 52 |
| 1.56 μM | 70 | 78 | 61 | 80 | 72 | 64 | 66 |
| 0.78 μM | 78 | 77 | 76 | 84 | 76 | 79 | 74 |
| 0.39 μM | 83 | 95 | 82 | 90 | 85 | 94 | 81 |
| 0.20 μM | 91 | 92 | 84 | 88 | 103 | 91 | 84 |

### HIF1A Expression in A549 (Average of Biological Replicate)

| | #1 | #2 | #3 | #4 | #5 | #6 | Reference |
|---|---|---|---|---|---|---|---|
| 25.00 μM | 31 | 33 | 30 | 42 | 36 | 37 | 32 |
| 12.50 μM | 45 | 49 | 43 | 58 | 50 | 55 | 48 |
| 6.25 μM | 62 | 65 | 64 | 82 | 74 | 75 | 70 |
| 3.13 μM | 82 | 83 | 81 | 88 | 88 | 101 | 88 |
| 1.56 μM | 88 | 87 | 94 | 95 | 100 | 105 | 97 |
| 0.78 μM | 92 | 106 | 99 | 102 | 97 | 102 | 97 |
| 0.39 μM | 96 | 98 | 102 | 103 | 99 | 106 | 102 |
| 0.20 μM | 96 | 94 | 97 | 95 | 97 | 103 | 99 |

### Example 4: In Vitro Potency and Efficacy of Oligonucleotides Targeting MALAT1 mRNA in Human HeLa and A549 Cells at Different Concentrations for a Dose-Response Curve

HeLa and A549 cell lines were purchased from ATCC and maintained as recommended by the supplier in a humidified incubator at 37° C. with 5% CO₂. For assays, 3000 cells/well (HeLa) and 3500 cells/well (A549) were seeded in a 96 multi well plate in culture media. Cells were incubated for 24 hours before addition of oligonucleotides dissolved in PBS. Concentration range of oligonucleotides: highest concentration 25 μM, 1:1 dilutions in 8 steps. Three days after addition of oligonucleotides, the cells were harvested. RNA was extracted using the PureLink Pro 96 RNA Purification kit (Thermo Fisher Scientific) according to the manufacturer's instructions and eluted in 50 μl water. The RNA was subsequently diluted 10 times with DNase/RNase free Water (Gibco) and heated to 90° C. for one minute.

For gene expressions analysis, One Step RT-qPCR was performed using qScript™ XLT One-Step RT-qPCR ToughMix®, Low ROX™ (Quantabio) in a duplex set up. The following TaqMan primer assays were used for qPCR: MALAT1, Hs00273907_s1 (FAM-MGB) with endogenous control GAPDH. All primer sets were purchased from Thermo Fisher Scientific. The relative expression level of MALAT1 mRNA is shown as percent of control (PBS-treated cells) and IC₅₀ values have been determined using GraphPad Prism7 on data from n=2 biological replicates.

The results are shown in the tables below and in FIGS. 1 and 2.

| Compound | IC₅₀ in | | IC₅₀ in | |
|---|---|---|---|---|
| ID No. | HeLa (μM) | SD | A549 (μM) | SD |
| Control | 0.44 | 0.06 | 0.79 | 0.11 |
| #7 | 0.34 | 0.07 | 0.59 | 0.06 |
| #8 | 0.28 | 0.05 | 0.61 | 0.05 |
| #9 | 0.31 | 0.03 | 0.62 | 0.05 |
| #10 | 0.20 | 0.03 | 0.47 | 0.08 |
| #11 | 0.22 | 0.01 | 0.49 | 0.07 |
| #12 | 0.29 | 0.02 | 0.43 | 0.05 |

The data depicted in the plots of FIGS. 1 and 2 is reported in the table below.

### MALAT1 Expression in HeLa (Average of Biological Replicate):

| | #7 | #8 | #9 | #10 | #11 | #12 | Reference |
|---|---|---|---|---|---|---|---|
| 25.00 μM | 5 | 4 | 3 | 3 | 3 | 3 | 6 |
| 12.50 μM | 6 | 5 | 4 | 3 | 3 | 4 | 7 |
| 6.25 μM | 9 | 7 | 7 | 5 | 4 | 5 | 9 |
| 3.13 μM | 13 | 13 | 8 | 7 | 7 | 8 | 15 |
| 1.56 μM | 23 | 22 | 14 | 10 | 12 | 13 | 22 |
| 0.78 μM | 29 | 27 | 32 | 19 | 19 | 20 | 37 |
| 0.39 μM | 49 | 40 | 35 | 32 | 40 | 37 | 64 |
| 0.20 μM | 73 | 65 | 77 | 64 | 67 | 70 | 79 |

### MALAT1 Expression in A549HeLa (Average of Biological Replicate)

| | #7 | #8 | #9 | #10 | #11 | #12 | Reference |
|---|---|---|---|---|---|---|---|
| 25.00 μM | 8 | 7 | 5 | 5 | 5 | 4 | 12 |
| 12.50 μM | 9 | 9 | 7 | 7 | 6 | 6 | 14 |
| 6.25 μM | 13 | 11 | 11 | 10 | 10 | 9 | 18 |
| 3.13 μM | 22 | 18 | 18 | 14 | 14 | 13 | 27 |
| 1.56 μM | 31 | 32 | 30 | 25 | 24 | 22 | 38 |
| 0.78 μM | 45 | 44 | 43 | 35 | 38 | 37 | 51 |
| 0.39 μM | 64 | 66 | 67 | 56 | 57 | 50 | 71 |
| 0.20 μM | 80 | 86 | 90 | 79 | 76 | 79 | 96 |

### Example 5: In Vitro Potency and Efficacy of Oligonucleotides Targeting ApoB mRNA in Mouse Primary Hepatocytes

Primary mouse hepatocytes were isolated from livers of C57BL/6J mice anesthetized with Pentobarbital after a 2 step perfusion protocol according to the literature (Berry and Friend, 1969, J. Cell Biol; Paterna et al., 1998, Toxicol. Appl. Pharmacol.). The first step was 5 min with HBSS+15 mM HEPES+0.4 mM EGTA followed by 12 min HBSS+20 mM NaHCO 3+0.04% BSA (Sigma #A7979)+4 mM CaCL 2 (Sigma #21115)+0.2 mg/ml Collagenase Type 2 (Worthington #4176). The Hepatocytes were captured in 5 ml cold Williams medium E (WME) (Sigma #W1878, complemented with 1× Pen/Strep/Glutamine, 10% (v/v) FBS (ATCC #30-2030)) on ice. The crude cell suspension was filtered through a 70 μm followed by a 40 μm cell strainer (Falcon #352350 and 15 #352340), filled up to 25 ml with WME and centrifuged at room temperature for 5 min at 50×g to pellet the hepatocytes. The supernatant was removed and the hepatocytes were resuspended in 25 ml WME. After adding 25 ml 90% Percoll solution (Sigma #P4937; pH=8.5-9.5) and centrifugation for 10 min at 25° C., 50×g the supernatant and floating cells were removed. To remove the remaining Percoll the pellet was resuspended again in 50 mL WME medium, centrifuged 3 min, 25° C. at 50×g and the supernatant discarded. The cell pellet was resuspended in 20 mL WME and cell number and viability determined (Invitrogen, Cellcount) and diluted to 250,000 cells/ml. 25,000 cells/well were seeded on collagen-coated 96-well plates (PD Biocoat Collagen I #356407) and incubated at 37° C., 5% C02. After 3 h, the cells were washed with WME to remove unattached cells and the medium was replaced. 24 h after seeding, oligonucleotides were added at a range of concentrations: highest concentration 3,125 μM, half-log dilutions in 8 steps. Three days after addition of oligonucleotides, the cells were harvested. RNA was extracted using the PureLink Pro 96 RNA Purification kit (Thermo Fisher Scientific) according to the manufacturer's instructions and eluted in 50 μl water. The RNA was subsequently diluted 10 times with DNase/RNase free Water (Gibco) and heated to 90° C. for one minute. For gene expressions analysis, One Step RT-qPCR was performed using qScript™ XLT One-Step RT-qPCR ToughMix®, Low ROX™ (Quantabio) in a duplex set up. The following TaqMan primer assays were used for qPCR: Apob Mm_01545150_m1 (FAM-MGB) with endogenous control Gapdh, Mm99999915_g1 (VIC-MGB). All primer sets were purchased from Thermo Fisher Scientific. The relative expression level of ApoB mRNA is shown as percent of control (PBS-treated cells) and IC50 values have been determined using GraphPad Prism7.

The results are shown in the tables below and in FIG. 5.

| Compound | IC₅₀ |
|---|---|
| ID No. | (uM, N = 2) |
| Control | 0.07 |
| #13 | 0.10 |
| #14 | 0.23 |
| #15 | 0.23 |
| #16 | 0.21 |
| #17 | 0.39 |
| #18 | 0.39 |
| #19 | 0.29 |
| #20 | 0.19 |
| #21 | 0.17 |
| #22 | 0.21 |
| #23 | 0.75 |

The data depicted in the plot of FIG. 5 is reported in the table below.

Relative Expression of ApoB mRNA in Primary Mouse Hepatocytes

| | #13 | #14 | #15 | #16 | #17 | #18 | #19 | #20 | #21 | #22 | #23 | Ref. |
|---|---|---|---|---|---|---|---|---|---|---|---|---|
| 3.125 μM | 13 | 11 | 12 | 13 | 12 | 16 | 13 | 11 | 11 | 11 | 18 | 16 |
| 0.989 μM | 12 | 13 | 14 | 13 | 18 | 22 | 20 | 13 | 14 | 14 | 24 | 15 |
| 0.313 μM | 16 | 19 | 22 | 19 | 27 | 30 | 28 | 20 | 24 | 26 | 42 | 26 |
| 0.099 μM | 25 | 42 | 44 | 41 | 59 | 56 | 43 | 42 | 40 | 33 | 62 | 34 |
| 0.031 μM | 54 | 73 | 72 | 75 | 79 | 86 | 81 | 67 | 60 | 76 | 75 | 44 |
| 0.010 μM | 73 | 81 | 86 | 83 | 89 | 88 | 86 | 74 | 113 | 127 | 89 | 69 |
| 0.003 μM | 94 | 87 | 92 | 86 | 86 | 86 | 85 | 104 | 108 | 89 | 83 | 88 |
| 0.001 μM | 94 | 108 | 110 | 117 | 120 | 111 | 102 | 96 | 89 | 83 | 91 | 88 |

### Example 6: Thermal Melting (Tm) of Oligonucleotides Containing a Phosphonoacetic Acid Internucleoside Linkage Hybridized to RNA and DNA

The denaturation point of dsLNA/DNA or dsLNA/RNA heteroduplexes (thermal melting=Tm) were measured according to the following procedure:

A solution of equimolar amount of RNA or DNA and LNA oligonucleotide (20 μM for ApoB and 10 μM for Malat-1) result in 10 μM dsOligonucleotide (ApoB) and 5 μM dsOligonucleotide (Malat-1) in buffer (137 mM NaCl, 2.7 mM KCl, 10 mM Na₂HPO₄, pH 7.4). The solutions were heated to 95° C. for 2 min (Hybridization) and then allowed to cool down to room temperature for 15 min. The UV absorbance at 260 nm was recorded using Evolution 600 UV-Vis spectrophotometer from Thermo Scientific (heating rate 1° C. per minute; reading rate twenty per min). For the determination of the denaturation point (i.e. melting points, Tm) the melting transition was fit with a LOWESS curve and the inflection point (=Tm) was identified by the peak position of the first derivative of the descriptive fit.

Tm measurements (RNA and DNA) for ApoB oligonucleotides are shown in the following table.

| | | Tm DNA | Tm RNA |
|---|---|---|---|
| Compound | Sequence | (° C.) | (° C.) |
| #13 | G^{m}CattggtatT*^{m}CA | 57.5 | 65.8 |
| #14 | G^{m}C*attggtatT^{m}CA | 58.4 | 65.9 |
| #15 | G^{m}CattggtatT^{m}C*A | 58.3 | 65.9 |
| #16 | G*^{m}CattggtatT^{m}CA | 57.3 | 67.5 |
| #17 | G*^{m}CattggtatT^{m}C*A | 57.7 | 65.7 |
| #18 | G^{m}C*attggtatT*^{m}CA | 55.2 | 65.7 |
| #19 | G^{m}C*attggtatT^{m}C*A | 55.4 | 65.8 |
| #20 | G*^{m}C*attggtatT^{m}CA | 55.5 | 65.8 |
| #21 | G^{m}CattggtatT*^{m}C*A | 55.9 | 66.2 |
| #22 | G*^{m}CattggtatT*^{m}CA | 54.0 | 65.7 |
| #23 | G*^{m}C*attggtatT*^{m}CA | 51.0 | 62.1 |
| Control | G^{m}CattggtatT^{m}CA | 58.8 | 69.1 |

The compounds according to the invention retain the high affinity for RNA and DNA of the control.

### Example 7: In Vitro Potency and Efficacy of Selected Oligonucleotides Targeting MALAT1 mRNA in LTK Cells (Fibroblasts)

The following oligonucleotides have been generated and tested accordingly:

| Compound | | Calculated | Found |
|---|---|---|---|
| ID No. | Sequence | mass | mass |
| #24 | GAGttacttgcca*A^{m}CT | 5321.3 | 5321.7 |
| #25 | GAGt*tacttgcca*A^{m}CT | 5363.3 | 5363.4 |
| #26 | GAGt^{o}tacttgcca^{o}A^{m}CT | 5331.3 | 5331.9 |
| #27 | GAGttacttgcca^{o}A^{m}CT | 5305.2 | 5304.9 |

*PACE phosphorothioate modification between adjacent nucleotides
^{o}PACE phosphorodiester modification between adjacent nucleotides
A, G, ^{m}C, T represent LNA nucleotides
a, g, c, t represent DNA nucleotides
all other linkages were prepared as phosphorothioates

| Compound | IC₅₀ in LTK |
|---|---|
| ID No. | cells (nM) N = 2 |
| Control | 138/165/188 |
| #24 | 172 |
| #25 | 142 |
| #26 | 202 |
| #27 | 121 |

The above compounds which target Malat-1 were tested in mouse fibroblasts (LTK cells) using gymnotic uptake for 72 hours, at a range of concentrations to determine the compound potency (IC50).

Concentration range for LTK cells: 50 μM, ½ log dilution, 8 concentrations. RNA levels of Malat1 were quantified using qPCR (Normalised to GAPDH level) and IC50 values were determined.

The IC50 results are shown in the above table, indicating that this chemical modification is well-tolerated in terms of target knockdown (as exemplified here for disease relevant skeletal muscle cells).

### Example 8: Measurement of Target mRNA Levels (Malat1) in Heart with a Dose of 15 Mg/Kg

Mice (C57/BL6) were administered 15 mg/kg dose subcutaneously of the oligonucleotide in three doses on day 1, 2 and 3 (n=5). The mice were sacrificed on day 8, and MALAT-1 RNA reduction was measured for the heart. The parent compound was administered in two doses 3*15 mg/kg and 3*30 mg/kg.

The results are shown in FIG. 6.

The in vivo results illustrate that the Thio-PACE modified compound #24 is about twice as potent in knocking down MALAT-1 in the heart as the reference compound (same efficacy at 15 mg/kg as the reference at 30 mg/kg dosing). Compound #25 which has an additional thio-PACE modification introduced at position 12 shows a lower efficacy than #24 but is still better than the reference. The corresponding Oxo-PACE analogue (#26) shows substantially reduced activity.

A major impact on efficacy has been observed in vivo with the single-stranded antisense oligonucleotide according to the invention. It should be noted that the dose of the oligonucleotide according to the invention is only 50% of the reference dose.

### Example 9: MOE PACE Monomer Synthesis

### 9.1. 1-cyano-2-methylpropan-2-yl 2-bromoacetate

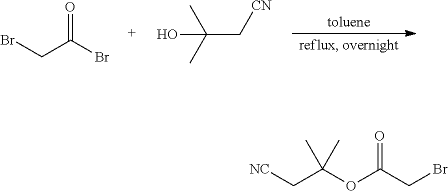

To a solution of 2-bromoacetyl bromide (14.7 g, 6.31 mL, 72.6 mmol, Eq: 1.2) was added to a 250 mL round-bottom flask containing toluene (67.2 mL). 3-hydroxy-3-methylbutanenitrile (6 g, 6.28 ml, 60.5 mmol, Eq: 1) was slowly added with stirring. The round-bottom flask was fitted with a Friedrich's condenser and a drying tube vented to an acid trap (containing NaOH aq.). The reaction mixture was heated to reflux and refluxed overnight. The reaction was allowed to cool down to room temperature and the mixture was then concentrated in vacuo to an oil. The crude oil was purified by Combiflash Chromatography using ethyl acetate/hexane as gradients: the product was eluted at 30% ethyl acetate in hexane to afford 1-cyano-2-methylpropan-2-yl 2-(bis(diisopropylamino)phosphaneyl)acetate (8.14 g, 37 mmol, 58% yield). ¹H NMR (CHLOROFORM-d, 300 MHz) δ 3.8 (s, 2H), 2.9 (s, 2H), 1.6 (s, 6H).

### 9.2. 1-cyano-2-methylpropan-2-yl 2-(bis(diisopropylamino)phosphaneyl)acetate

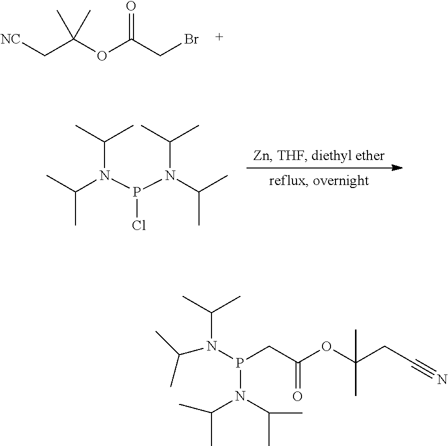

Anhydrous THF (69.4 ml), l-chloro-N,N,N′,N′-tetraisopropylphosphanediamine (7.75 g, 29 mmol, Eq: 1) and a magnetic stir bar were added to a 250 mL round-bottom flask which was stoppered, and the solution was allowed to be stirred until the phosphine dissolved. After dissolution, anh. diethyl ether (41.6 ml) was added. 1-cyano-2-methylpropan-2-yl 2-bromoacetate (7.03 g, 32 mmol, Eq: 1.1) was placed in a 100 mL round-bottom flask, and anh. THF (34.7 ml) was added. Zinc (2.85 g, 43.6 mmol, Eq: 1.5), anh. diethyl ether (22.2 ml) and a magnetic stir bar were placed in a 500 mL three necked round-bottom flask fitted with a Friedrich's condenser. The phosphine (36 mL) and the bromoacetate solutions (10 mL) were added to the three necked round-bottom flask. The reaction mixture was then heated under reflux until an exothermic reaction was noticeable (the slightly cloudy, colorless reaction became clear and slightly yellow). The reaction was continued at reflux by the addition of the remainder of the phosphine and bromoacetate solutions. Once the addition was complete, the reaction was kept at reflux for 45 min by heating, allowed to cool down to room temperature and analyzed for completeness by ³¹P NMR. The starting material at δ=135 ppm was converted to a single product at δ=48 ppm. The cooled reaction mixture was concentrated in vacuo to a viscous oil. The resulting viscous oil was dissolved with anhydrous heptane. The formed solid was then dissolved in acetonitrile, and this solution was extracted twice with anh. heptane. The acetonitrile solution was analyzed by ³¹P NMR for absence of the product at δ=48 ppm and discarded. All heptane fractions were combined (top layer) and concentrated in vacuo to give a slightly yellow oil. It was then dried under high vacuum overnight. After drying overnight, the product obtained was a nice white solid (7.096 g, 19 mmol, 62% yield). ¹H NMR (CHLOROFORM-d, 300 MHz) δ 3.3-3.5 (m, 4H), 2.9 (s, 2H), 2.7 (d, 2H), 1.60 (s, 6H), 1.3 (m, 24H).

### 9.3. (1-cyano-2-methylpropan-2-yl) 2-[[di(propan-2-yl)amino]-[rac-(2R,5R)-2-[[bis(4-methoxyphenyl)-phenylmethoxy]methyl]-4-(2-methoxy ethoxy)-5-(5-methyl-2,4-dioxopyrimidin-1-yl)oxolan-3-yl]oxyphosphanyl]acetate

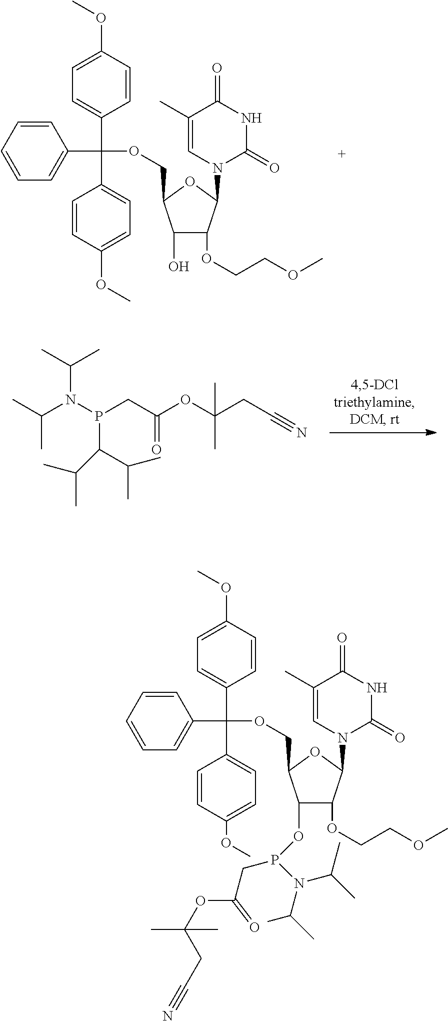

5-methyl-1-[rac-(2R,5R)-4-hydroxy-3-(2-methoxyethoxy)-5-[[rac-(2E)-1,1-bis(4-methoxyphenyl)-2-[rac-(Z)-prop-1-enyl]penta-2,4-dienoxy]methyl]oxolan-2-yl]pyrimidine-2,4-dione (800 mg, 1.29 mmol, Eq: 1) was dissolved in anh. DCM (16.2 ml), 1-cyano-2-methylpropan-2-yl 2-(bis(diisopropylamino)phosphaneyl)acetate (721 mg, 1.94 mmol, Eq: 1.5) was then added to the reaction mixture. Upon complete dissolution of the reaction components, 4,5-DCI (122 mg, 1.03 mmol, Eq: 0.8) was added to the reaction mixture. The reaction mixture was then allowed to stir at room temperature overnight under argon and analyzed for the extent of the reaction by ³¹P NMR and silica gel TLC (eluted with ethyl acetate). The reaction was determined to be complete by spot to spot conversion to a faster eluting product on TLC and by a complete loss of the acetic acid phosphinodiamite ³¹P NMR signal. Upon completion, the reaction was quenched by the addition of triethylamine (105 mg, 144 μl, 1.03 mmol, Eq: 0.8). After 5 min, the reaction mixture was concentrated to a viscous oil in vacuo using a rotavap. The viscous oil was redissolved in a minimum volume of ethyl acetate and was added to the top of a silica gel column preequilibrated with 80/20: ethyl acetate/heptane to collect the product. The fractions containing the product were combined and concentrated to a foam in vacuo on a rotavap, redissolved in a minimal amount of anh. DCM, and added dropwise to rapidly stirring anh. heptane. The solid precipitate was isolated by filtration and dried overnight in vacuo to afford 736 mg of target compound as a white solid (736 mg, 61% yield). LCMS (ES+) found: 889.5 g/mol.

### 9.4. (1-cyano-2-methylpropan-2-yl) 2-[[di(propan-2-yl)amino]-[rac-(2R,5R)-5-(6-benzamidopurin-9-yl)-2-[[bis(4-methoxyphenyl)-phenylmethoxy]methyl]-4-(2-methoxyethoxy)oxolan-3-yl]oxyphosphanyl]acetate

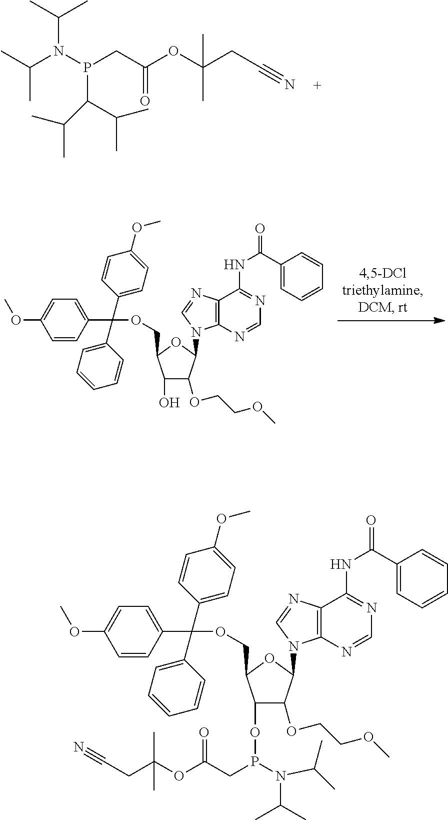 [SMILES: CC(C)C(C(C)C)P(CC(=O)OC(C)(C)CC#N)N(C(C)C)C(C)C ; COCCOC1C(O)[C@@H](COC(c2ccccc2)(c2ccc(OC)cc2)c2ccc(OC)cc2)O[C@H]1n1cnc2c(NC(=O)c3ccccc3)ncnc21 ; COCCOC1C(OP(CC(=O)OC(C)(C)CC#N)N(C(C)C)C(C)C)[C@@H](COC(c2ccccc2)(c2ccc(OC)cc2)c2ccc(OC)cc2)O[C@H]1n1cnc2c(NC(=O)c3ccccc3)ncnc21]

Rac-N-(9-((2R,5R)-5-((bis(4-methoxyphenyl)(phenyl)methoxy)methyl)-4-hydroxy-3-(2-methoxyethoxy)tetrahydrofuran-2-yl)-9H-purin-6-yl)benzamide (600 mg, 0.82 mmol, Eq: 1) was dissolved in anh. DCM (10.2 ml), 1-cyano-2-methylpropan-2-yl 2-(bis(diisopropylamino)phosphaneyl)acetate (457 mg, 1.23 mmol, Eq: 1.5) was then added to the reaction mixture. Upon complete dissolution of the reaction components, 4,5-DCI (77.5 mg, 0.66 mmol, Eq: 0.8) was added to the reaction mixture. The reaction mixture was then allowed to stir at room temperature overnight under argon and analyzed for the extent of the reaction by ³¹P NMR and silica gel TLC (eluted with ethyl acetate). The reaction was determined to be complete by spot to spot conversion to a faster eluting product on TLC and by a complete loss of the acetic acid phosphinodiamite ³¹P NMR signal. Upon completion, the reaction was quenched by the addition of triethylamine (66.4 mg, 91.4 μl, 0.65 mmol, Eq: 0.8). After 5 min, the reaction mixture was concentrated to a viscous oil in vacuo using a rotavap. The viscous oil was redissolved in a minimum volume of ethyl acetate and was added to the top of a silica gel column preequilibrated with 80/20: ethyl acetate/heptane to collect the product. The fractions containing the product were combined and concentrated to a foam in vacuo on a rotavap, redissolved in a minimal amount of anh. DCM, and added dropwise to rapidly stirring anh. heptane. The solid precipitate was isolated by filtration and dried overnight in vacuo to afford 260 mg of target compound as a white solid (260 mg, 32% yield). LCMS (ES+) found: 1002.5 g/mol.

### 9.5. (1-cyano-2-methylpropan-2-yl) 2-[[di(propan-2-yl)amino]-[rac-(2R,5R)-2-[[bis(4-methoxyphenyl)-phenylmethoxy]methyl]-4-(2-methoxy ethoxy)-5-[2-(2-methylpropanoylamino)-6-oxo-1H-purin-9-yl]oxolan-3-yl]oxyphosphanyl]acetate

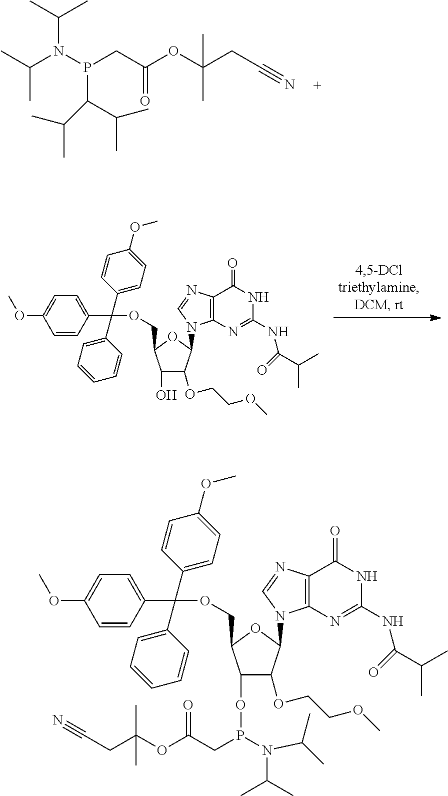

2-methyl-N-[6-oxo-9-[rac-(2R,5R)-5-[[bis(4-methoxyphenyl)-phenylmethoxy]methyl]-4-hydroxy-3-(2-methoxyethoxy)oxolan-2-yl]-1H-purin-2-yl]propanamide (700 mg, 0.98 mmol, Eq: 1) was dissolved in anh. DCM (12.3 ml), 1-cyano-2-methylpropan-2-yl 2-(bis(diisopropylamino)phosphaneyl)acetate (546 mg, 1.47 mmol, Eq: 1.5) was then added to the reaction mixture. Upon complete dissolution of the reaction components, 4,5-DCI (93 mg, 0.79 mmol, Eq: 0.8) was added to the reaction mixture. The reaction mixture was then allowed to stir at room temperature overnight under argon and analyzed for the extent of the reaction by ³¹P NMR and silica gel TLC (eluted with ethyl acetate). The reaction was determined to be complete by spot to spot conversion to a faster eluting product on TLC and by a complete loss of the acetic acid phosphinodiamite ³¹P NMR signal. Upon completion, the reaction was quenched by the addition of triethylamine (80 mg, 109 μl, 0.79 mmol, Eq: 0.8). After 5 min, the reaction mixture was concentrated to a viscous oil in vacuo using a rotavap. The viscous oil was redissolved in a minimum volume of ethyl acetate and was added to the top of a silica gel column preequilibrated with ethyl acetate to collect the product. The fractions containing the product were combined and concentrated to a foam in vacuo on a rotavap, redissolved in a minimal amount of anh. DCM, and added dropwise to rapidly stirring anh. heptane. The solid precipitate was isolated by filtration and dried overnight in vacuo to afford 520 mg of target compound as a white solid (520 mg, 49% yield). LCMS (ES+) found: 984.5 g/mol.

### 9.6. (1-cyano-2-methylpropan-2-yl) 2-[[di(propan-2-yl)amino]-[rac-(2R,5R)-5-(4-benzamido-5-methyl-2-oxopyrimidin-1-yl)-2-[[bis(4-methoxyphenyl)-phenylmethoxy]methyl]-4-(2-methoxyethoxy)oxolan-3-yl]oxyphosphanyl]acetate

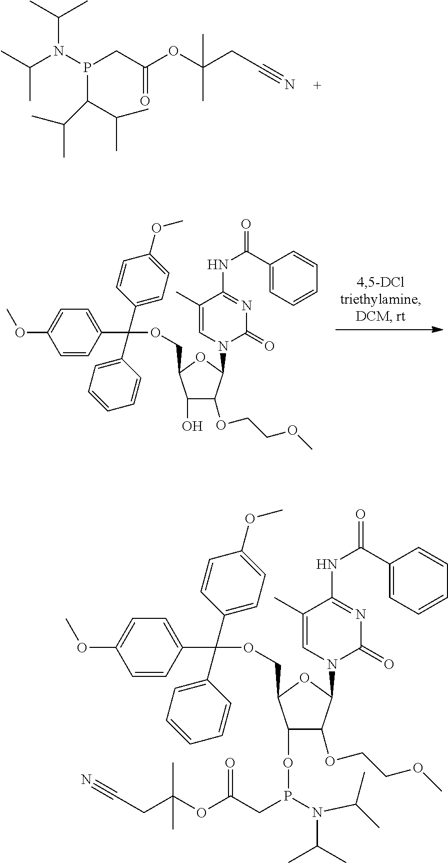

N-[5-methyl-2-oxo-1-[rac-(2R,5R)-5-[[bis(4-methoxyphenyl)-phenylmethoxy]methyl]-4-hydroxy-3-(2-methoxyethoxy)oxolan-2-yl]pyrimidin-4-yl]benzamide (950 mg, 1.32 mmol, Eq: 1) was dissolved in anh. DCM (16.5 ml), 1-cyano-2-methylpropan-2-yl 2-(bis(diisopropylamino)phosphaneyl)acetate (733 mg, 1.97 mmol, Eq: 1.5) was then added to the reaction mixture. Upon complete dissolution of the reaction components, 4,5-DCI (124 mg, 1.05 mmol, Eq: 0.8) was added to the reaction mixture. The reaction mixture was then allowed to stir at room temperature overnight under argon and analyzed for the extent of the reaction by ³¹P NMR and silica gel TLC (eluted with ethyl acetate). The reaction was determined to be complete by spot to spot conversion to a faster eluting product on TLC and by a complete loss of the acetic acid phosphinodiamite ³¹P NMR signal. Upon completion, the reaction was quenched by the addition of triethylamine (107 mg, 147 μl, 1.05 mmol, Eq: 0.8). After 5 min, the reaction mixture was concentrated to a viscous oil in vacuo using a rotavap. The viscous oil was redissolved in a minimum volume of ethyl acetate and was added to the top of a silica gel column preequilibrated with 80/20: ethyl acetate/heptane to collect the product. The fractions containing the product were combined and concentrated to a foam in vacuo on a rotavap, redissolved in a minimal amount of anh. DCM, and added dropwise to rapidly stirring anh. heptane. The solid precipitate was isolated by filtration and dried overnight in vacuo to afford 722 mg of target compound as a light yellow solid (722 mg, 55% yield). LCMS (ES+) found: 992.4 g/mol.

### Example 10: Oligonucleotide Synthesis

Oligonucleotides were synthesized using a MerMade 12 automated DNA synthesizer by Bioautomation. Syntheses were conducted on a 1 μmol scale using a controlled pore glass support (500 Å) bearing a universal linker.

In standard cycle procedures for the coupling of standard DNA and LNA phosphoramidites DMT deprotection was performed with 3% (w/v) dichloroacetic acid in CH₂Cl₂ in three applications of 230 μL for 105 sec. The respective phosphoramidites were coupled three times with 95 μL of 0.1M solutions in acetonitrile (or acetonitrile/CH₂C₁₋₂ —1:1 for the LNA-^{Me}C building block) and 110 μL of a 0.25M solution of 5-[3,5-Bis(trifluoromethyl)phenyl]-2H-tetrazole as an activator and a coupling time of 180 sec. Sulfurization was performed using a 0.1M solution of 3-amino-1,2,4-dithiazole-5-thione in acetonitrile/pyridine in one application of 200 μL for 3 minutes. Oxidation was performed using a 0.02M I₂ in THF/pyr/H₂O:88/10/2 in one application for 3 minutes. Capping was performed using THF/lutidine/Ac₂O 8:1:1 (CapA, 75 μmol) and THF/N-methyl imidazole 8:2 (CapB, 75 μmol) for 70 sec.

Synthesis cycles for the introduction of MOE PACE included DMT deprotection using 3% (w/v) dichloroacetic acid in in CH₂Cl₂ in three applications of 230 μL for 105 sec. Freshly prepared MOE PACE phosphoramidites were coupled two times with 95 μL of 0.1M solution in acetonitrile and 110 μL of a 0.25M solution of 5-[3,5-Bis(trifluoromethyl)phenyl]-2H-tetrazole as an activator and a coupling time of 15 minutes. Sulfurization was performed using a 0.1M solution of 3-amino-1,2,4-dithiazole-5-thione in acetonitrile/pyridine in one application for 3 minutes. Oxidation was performed using a 0.02M I₂ in THF/pyr/H₂O:88/10/2 in one application for 3 minutes. Capping was performed using THF/lutidine/Ac₂O 8:1:1 (CapA, 75 μmol) and THF/N-methyl imidazole 8:2 (CapB, 75 μmol) for 70 sec.

After the synthesis, a solution of 1.5% DBU in anh. CH₃CN was carefully passed through the column a few times to deprotect the dimethylcyanoethyl protecting groups and to prevent alkylation of the bases during deprotection. It was then allowed to stand at RT for 60 minutes. The solution was then discarded and the column was rinsed with 2-3 mL of anh. CH₃CN. It was then dried under stream of argon. The CPG was then transferred carefully into a 4 mL vial where 1 mL of 40% MeNH₂ in water was added and left under stirring for 15 min at 55° C.

Crude DMT-on oligonucleotides were purified by RP-HPLC purification using a C18 column followed by DMT removal with 80% aqueous acetic acid and ethanol precipitation or by cartridge purification. The MOE PACE phosphoramidites were synthesized in Basel. The normal phosphoramidites were ordered from Sigma Aldrich, as well as all of the reagents used in the solid phase synthesis.

### Example 11: In Vitro Potency and Efficacy of Oligonucleotides Targeting MALAT1 mRNA in Human HeLa Cells at Different Concentrations for a Dose-Response Curve

HeLa cell lines were purchased from ATCC and maintained as recommended by the supplier in a humidified incubator at 37° C. with 5% CO₂. For assays, 3000 cells/well were seeded in a 96 multi well plate in culture media. Cells were incubated for 24 hours before addition of oligonucleotides dissolved in PBS. Concentration range of oligonucleotides: highest concentration 25 μM, 1:1 dilutions in 8 steps. Three days after addition of oligonucleotides, the cells were harvested. RNA was extracted using the PureLink Pro 96 RNA Purification kit (Thermo Fisher Scientific) according to the manufacturer's instructions and eluted in 50 μl water. The RNA was subsequently diluted 10 times with DNase/RNase free Water (Gibco) and heated to 90° C. for one minute.

For gene expressions analysis, One Step RT-qPCR was performed using qScript™ XLT One-Step RT-qPCR ToughMix®, Low ROX™ (Quantabio) in a duplex set up. The following TaqMan primer assays were used for qPCR: MALAT1, Hs00273907_s1 (FAM-MGB) with endogenous control GAPDH. All primer sets were purchased from Thermo Fisher Scientific. The relative expression level of MALAT1 mRNA is shown as percent of control (PBS-treated cells) and IC₅₀ values have been determined using GraphPad Prism7 on data from n=2 biological replicates.

The results are provided in the following tables.

| | | Compound | | |
|---|---|---|---|---|
| Reference Sequence | IC50 [uM] | ID No. | Sequence | IC50 [uM] |
| GAGttacttgccaACT | 0.32 | | | |
| GAGt^{(ps)}tacttgccaACT | 2.06 | #28 | GAGt*tacttgccaACT | 0.38 |
| GAGtt^{(ps)}acttgccaACT | 1.95 | #29 | GAGtt*acttgccaACT | 0.57 |
| GAGtta^{(ps)}cttgccaACT | 0.19 | #30 | GAGtta*cttgccaACT | 0.33 |
| GAGttac^{(ps)}ttgccaACT | 0.40 | #31 | GAGttac*ttgccaACT | 0.83 |
| GAGttact^{(ps)}tgccaACT | 0.58 | #32 | GAGttact*tgccaACT | 1.07 |
| GAGttactt^{(ps)}gccaACT | 0.64 | #33 | GAGttactt*gccaACT | 0.48 |
| GAGttacttg^{(ps)}ccaACT | 0.93 | #34 | GAGttacttg*ccaACT | 1.89 |
| GAGttacttgc^{(ps)}caACT | 0.76 | #35 | GAGttacttgc*caACT | 0.86 |
| GAGttacttgcc^{(ps)}aACT | 0.51 | #36 | GAGttacttgcc*aACT | 0.44 |
| GAGttacttgcca^{(ps)}ACT | 0.60 | #37 | GAGttacttgcca*ACT | 0.23 |

| | | Compound | | |
|---|---|---|---|---|
| Reference Sequence | IC50 [uM] | ID No. | Sequence | IC50 [uM] |
| GAGttacttgccaACT | 0.32 | | | |
| GAGt^{(po)}tacttgccaACT | 1.97 | #38 | GAGt^{o}tacttgccaACT | 0.40 |
| GAGtt^{(po)}acttgccaACT | 2.19 | #39 | GAGtt^{o}acttgccaACT | 0.46 |
| GAGtta^{(po)}cttgccaACT | 0.29 | #40 | GAGtta^{o}cttgccaACT | 0.40 |
| GAGttac^{(po)}ttgccaACT | 0.68 | #41 | GAGttac^{o}ttgccaACT | 0.42 |
| GAGttact^{(po)}tgccaACT | 0.75 | #42 | GAGttact^{o}tgccaACT | 0.59 |
| GAGttactt^{(po)}gccaACT | 1.15 | #43 | GAGttactt^{o}gccaACT | 0.25 |
| GAGttacttg^{(po)}ccaACT | 1.85 | #44 | GAGttacttg^{o}ccaACT | 1.77 |
| GAGttacttgc^{(po)}caACT | 1.22 | #45 | GAGttacttgc^{o}caACT | 0.51 |
| GAGttacttgcc^{(po)}aACT | 0.37 | #46 | GAGttacttgcc^{o}aACT | 0.25 |
| GAGttacttgcca^{(po)}ACT | 0.46 | #47 | GAGttacttgcca^{o}ACT | 0.14 |

| | | Compound | | |
|---|---|---|---|---|
| Reference Sequence | IC50 [uM] | ID No. | Sequence | IC50 [uM] |
| GAGttacttgccaACT | 0.32 | | | |
| GAGttacttgccaAc^{(ps)}T | 0.14 | #48 | GAG*ttacttgccaAc*T | 0.07 |
| GAGttacttgccaa^{(ps)}CT | 0.12 | #49 | GAG*ttacttgccaa*CT | 0.11 |
| GAg^{(ps)}ttacttgccaACT | 0.27 | #50 | GAg*ttacttgccaACT | 0.11 |
| Ga^{(ps)}GttacttgccaACT | 0.40 | #51 | Ga*GttacttgccaACT | 0.21 |
| g^{(ps)}AGttacttgccaACT | 0.46 | #52 | g*AGttacttgccaACT | 0.86 |
| GAGttacttgccaAc^{(po)}T | 0.14 | #53 | GAGttacttgccaAc^{o}T | 0.11 |
| GAGttacttgccaa^{(po)}CT | 0.16 | #54 | GAGttacttgccaa^{o}CT | 0.19 |
| GAg^{(po)}ttacttgccaACT | 0.42 | #55 | GAg^{o}ttacttgccaACT | 0.14 |
| Ga^{(po)}GttacttgccaACT | 0.54 | #56 | Ga^{o}GttacttgccaACT | 0.52 |
| g^{(po)}AGttacttgccaACT | 0.58 | #57 | g^{o}AGttacttgccaACT | 0.60 |

Bold letters t, a, g, c represent MOE modifications.
(ps) phosphorothioate modification between adjacent nucleotides
(po) phosphorodiester modification between adjacent nucleotides
* PACE phosphorothioate modification between adjacent nucleotides
^{∘} PACE phosphorodiester modification between adjacent nucleotides
A, G, ^{m}C, T represent LNA nucleotides
a, g, c, t represent DNA nucleotides
all other linkages were prepared as phosphorothioates.

1. A compound of formula (I-a) 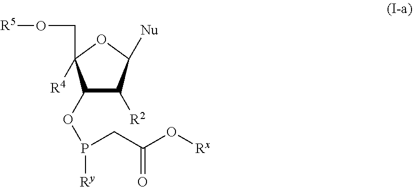
wherein
R² is methoxyethoxy; and
R⁴ is hydrogen; or
R⁴ and R² together form X-Y;
X is oxygen, sulfur, —CR^{a}R^{b}—, —C(R^{a})═C(R^{b})—, —C(═CR^{a}R^{b})—, —C(R^{a})═N—, —Si(R^{a})₂—, —SO₂—, —NR^{a}—; —O—NR^{a}—, —NR^{a}—O—, —C(=J)-, Se, —O—NR³—, —NR^{a}—CR^{a}R^{b}—, —N(R^{a})—O— or —O—CR^{a}R^{b}—;
Y is oxygen, sulfur, —(CR^{a}R^{b})ₙ—, —CR^{a}R^{b}—O—CR^{a}R^{b}—, —C(R^{a})═C(R^{b})—, —C(R^{a})═N—, —Si(R^{a})₂—, —SO₂—, —NR^{a}—, —C(=J)-, Se, —O—NR³—, —NR^{a}—CR^{a}R^{b}—, —N(R^{a})—O— or —O—CR^{a}R^{b}—;
with the proviso that —X—Y— is not —O—O—, Si(R^{a})₂—Si(R^{a})₂—, —SO₂—SO₂—, —C(R^{a})═C(R^{b})—C(R^{a})═C(R^{b}), —C(R^{a})═N—C(R^{a})═N—, —C(R^{a})═N—C(R^{a})═C(R^{b}), —C(R^{a})═C(R^{b})—C(R^{a})═N— or —Se—Se—;
J is oxygen, sulfur, ═CH₂ or ═N(R^{a});
R^{a} and R^{b} are independently selected from hydrogen, halogen, hydroxyl, cyano, thiohydroxyl, alkyl, substituted alkyl, alkenyl, substituted alkenyl, alkynyl, substituted alkynyl, alkoxy, substituted alkoxy, alkoxyalkyl, alkenyloxy, carboxyl, alkoxycarbonyl, alkylcarbonyl, formyl, aryl, heterocyclyl, amino, alkylamino, carbamoyl, alkylaminocarbonyl, aminoalkylaminocarbonyl, alkylaminoalkylaminocarbonyl, alkylcarbonylamino, carbamido, alkanoyloxy, sulfonyl, alkylsulfonyloxy, nitro, azido, thiohydroxylsulfidealkylsulfanyl, aryloxycarbonyl, aryloxy, arylcarbonyl, heteroaryl, heteroaryloxycarbonyl, heteroaryloxy, heteroarylcarbonyl, —OC(═X^{a})R^{c}, —OC(═X^{a})NR^{c}R^{d} and —NR^{e}C(═X^{a})NR^{c}R^{d};
or two geminal R^{a} and R^{b} together form optionally substituted methylene;
or two geminal R^{a} and R^{b}, together with the carbon atom to which they are attached, form cycloalkyl or halocycloalkyl, with only one carbon atom of —X—Y—;
wherein substituted alkyl, substituted alkenyl, substituted alkynyl, substituted alkoxy and substituted methylene are alkyl, alkenyl, alkynyl and methylene substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl, formyl, heterocylyl, aryl and heteroaryl;
X^{a} is oxygen, sulfur or —NR^{c};
R^{c}, R^{d} and R^{e} are independently selected from hydrogen and alkyl;
R⁵ is a hydroxyl protecting group;
R^{x} is cyanoalkyl or alkyl;
R^{y} is dialkylamino or pyrrolidinyl;
Nu is a nucleobase or a protected nucleobase; and
n is 1, 2 or 3;
or a pharmaceutically acceptable salt thereof.

2. A compound according to claim 1 of formula (II) 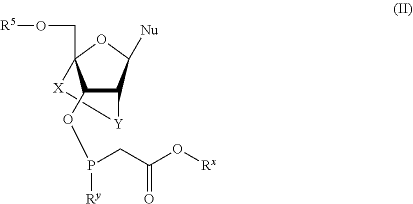
wherein
X is oxygen, sulfur, —CR^{a}R^{b}—, —C(R^{a})═C(R^{b})—, —C(═CR^{a}R^{b})—, —C(R^{a})═N—, —Si(R^{a})₂—, —SO₂—, —NR^{a}—; —O—NR³—, —NR^{a}—O—, —C(=J)-, Se, —O—NR³—, —NR^{a}—CR^{a}R^{b}—, —N(R^{a})—O— or —O—CR^{a}R^{b}—;
Y is oxygen, sulfur, —(CR^{a}R^{b})ₙ—, —CR^{a}R^{b}—O—CR^{a}R^{b}—, —C(R^{a})═C(R^{b})—, —C(R^{a})═N—, —Si(R^{a})₂—, —SO₂—, —NR^{a}—, —C(=J)-, Se, —O—NR³—, —NR^{a}—CR^{a}R^{b}—, —N(R^{a})—O— or —O—CR^{a}R^{b}—;
with the proviso that —X—Y— is not —O—O—, Si(R^{a})₂—Si(R^{a})₂—, —SO₂—SO₂—, —C(R^{a})═C(R^{b})—C(R^{a})═C(R^{b}), —C(R^{a})═N—C(R^{a})═N—, —C(R^{a})═N—C(R^{a})═C(R^{b}), —C(R^{a})═C(R^{b})—C(R^{a})═N— or —Se—Se—;
J is oxygen, sulfur, ═CH₂ or ═N(R^{a});
R^{a} and R^{b} are independently selected from hydrogen, halogen, hydroxyl, cyano, thiohydroxyl, alkyl, substituted alkyl, alkenyl, substituted alkenyl, alkynyl, substituted alkynyl, alkoxy, substituted alkoxy, alkoxyalkyl, alkenyloxy, carboxyl, alkoxycarbonyl, alkylcarbonyl, formyl, aryl, heterocyclyl, amino, alkylamino, carbamoyl, alkylaminocarbonyl, aminoalkylaminocarbonyl, alkylaminoalkylaminocarbonyl, alkylcarbonylamino, carbamido, alkanoyloxy, sulfonyl, alkylsulfonyloxy, nitro, azido, thiohydroxylsulfidealkylsulfanyl, aryloxycarbonyl, aryloxy, arylcarbonyl, heteroaryl, heteroaryloxycarbonyl, heteroaryloxy, heteroarylcarbonyl, —OC(═X^{a})R^{c}, —OC(═X^{a})NR^{c}R^{d} and —NR^{e}C(═X^{a})NR^{c}R^{d};
or two geminal R^{a} and R^{b} together form optionally substituted methylene;
or two geminal R^{a} and R^{b}, together with the carbon atom to which they are attached, form cycloalkyl or halocycloalkyl, with only one carbon atom of —X—Y—;
wherein substituted alkyl, substituted alkenyl, substituted alkynyl, substituted alkoxy and substituted methylene are alkyl, alkenyl, alkynyl and methylene substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl, formyl, heterocylyl, aryl and heteroaryl;
X^{a} is oxygen, sulfur or —NR^{c};
R^{c}, R^{d} and R^{e} are independently selected from hydrogen and alkyl;
R⁵ is a hydroxyl protecting group;
R^{x} is cyanoalkyl or alkyl;
R^{y} is dialkylamino or pyrrolidinyl;
Nu is a nucleobase or a protected nucleobase; and
n is 1, 2 or 3;
or a pharmaceutically acceptable salt thereof.

3. A compound according to claim 1 of formula (VI) 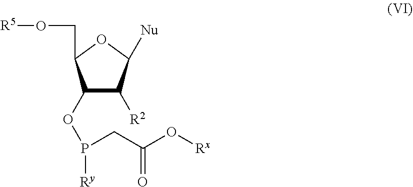
wherein R², R⁵, R^{x}, R^{y} and Nu are as defined in claim 1.

4. The compound according to claim 1, wherein —X—Y— is —CH₂—O—, —CH(CH₃)—O— or —CH₂CH₂—O—.

5. The compound according to claim 1 of formula (III), (IV) or (VII) 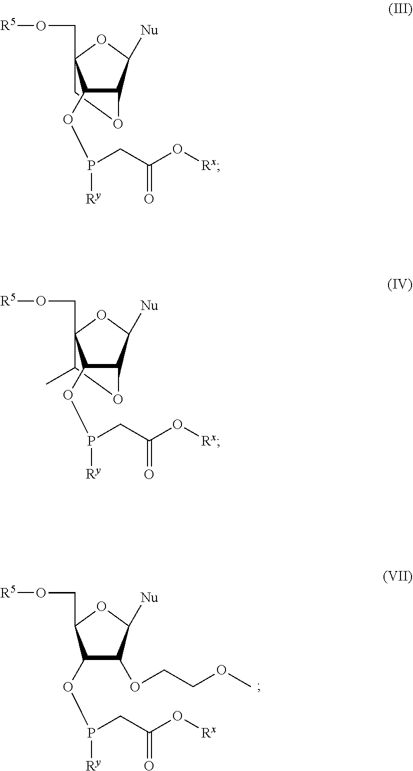
wherein R⁵, R^{x}, R^{y} and Nu are as defined in claim 1.

6. The compound according to claim 1, wherein R^{x} is 2-cyano-1,1-dimethyl-ethyl.

7. The compound according to claim 1, wherein R^{y} is diisopropylamino or pyrrolidinyl.

8. The compound according to claim 1, wherein R^{y} is dialkylamino.

9. The compound according to claim 1, wherein R^{y} is diisopropylamino.

10. The compound according to claim 1 of formula (V) or (VIM) 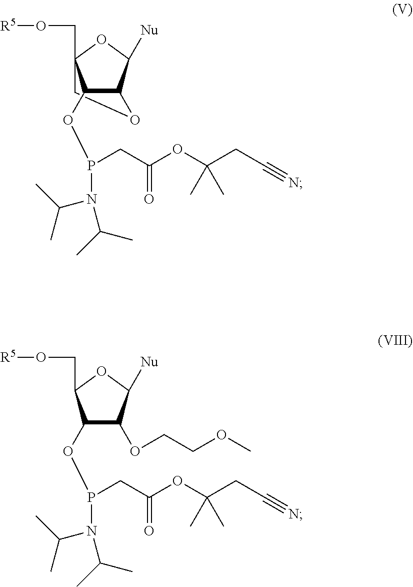
wherein R⁵ and Nu are as defined in claim 1.

11. The compound according to claim 1, wherein Nu is thymine, protected thymine, adenosine, protected adenosine, cytosine, protected cytosine, 5-methylcytosine, protected 5-methylcytosine, guanine, protected guanine, uracyl or protected uracyl.

12. The compound according to claim 1 selected from

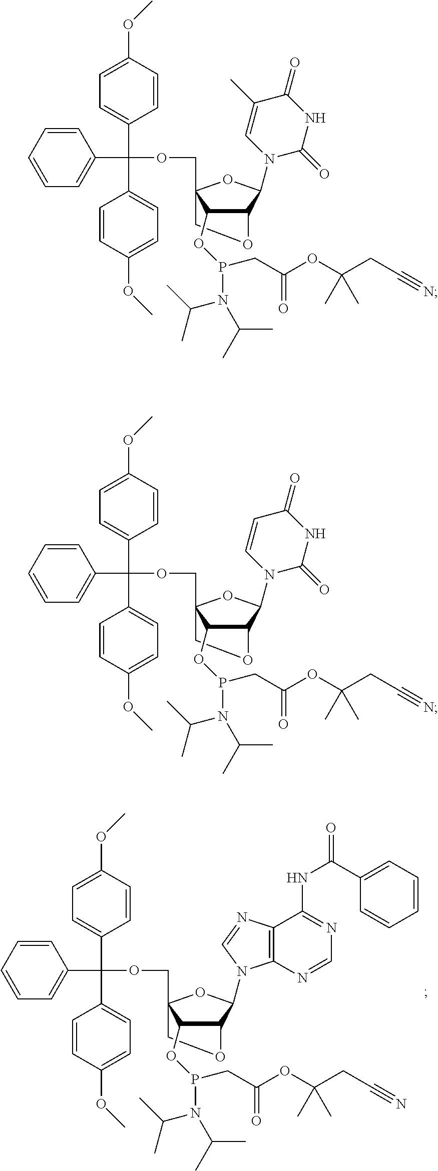 [SMILES: COc1ccc(C(OC[C@]23CO[C@@H](C2OP(CC(=O)OC(C)(C)CC#N)N(C(C)C)C(C)C)[C@H](n2cc(C)c(=O)[nH]c2=O)O3)(c2ccccc2)c2ccc(OC)cc2)cc1 ; COc1ccc(C(OC[C@]23CO[C@@H](C2OP(CC(=O)OC(C)(C)CC#N)N(C(C)C)C(C)C)[C@H](n2ccc(=O)[nH]c2=O)O3)(c2ccccc2)c2ccc(OC)cc2)cc1 ; COc1ccc(C(OC[C@]23CO[C@@H](C2OP(CC(=O)OC(C)(C)CC#N)N(C(C)C)C(C)C)[C@H](n2cnc4c(NC(=O)c5ccccc5)ncnc42)O3)(c2ccccc2)c2ccc(OC)cc2)cc1]

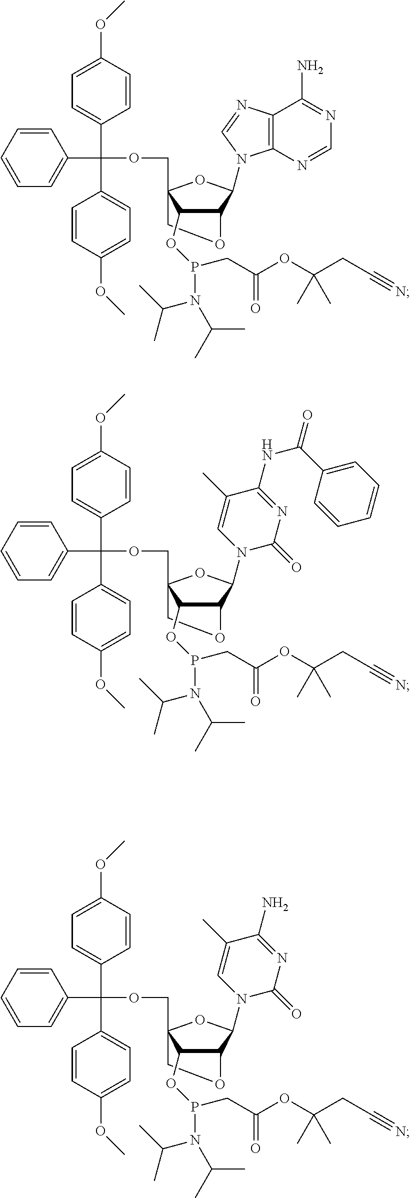 [SMILES: COc1ccc(C(OC[C@]23CO[C@@H](C2OP(CC(=O)OC(C)(C)CC#N)N(C(C)C)C(C)C)[C@H](n2cnc4c(N)ncnc42)O3)(c2ccccc2)c2ccc(OC)cc2)cc1 ; COc1ccc(C(OC[C@]23CO[C@@H](C2OP(CC(=O)OC(C)(C)CC#N)N(C(C)C)C(C)C)[C@H](n2cc(C)c(NC(=O)c4ccccc4)nc2=O)O3)(c2ccccc2)c2ccc(OC)cc2)cc1 ; COc1ccc(C(OC[C@]23CO[C@@H](C2OP(CC(=O)OC(C)(C)CC#N)N(C(C)C)C(C)C)[C@H](n2cc(C)c(N)nc2=O)O3)(c2ccccc2)c2ccc(OC)cc2)cc1]

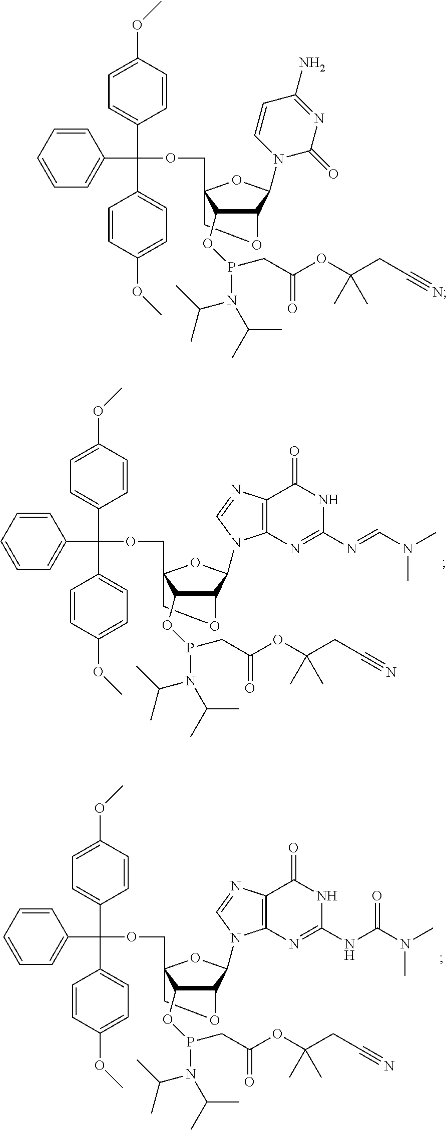 [SMILES: COc1ccc(C(OC[C@]23CO[C@@H](C2OP(CC(=O)OC(C)(C)CC#N)N(C(C)C)C(C)C)[C@H](n2ccc(N)nc2=O)O3)(c2ccccc2)c2ccc(OC)cc2)cc1 ; COc1ccc(C(OC[C@]23CO[C@@H](C2OP(CC(=O)OC(C)(C)CC#N)N(C(C)C)C(C)C)[C@H](n2cnc4c(=O)[nH]c(/N=C/N(C)C)nc42)O3)(c2ccccc2)c2ccc(OC)cc2)cc1 ; COc1ccc(C(OC[C@]23CO[C@@H](C2OP(CC(=O)OC(C)(C)CC#N)N(C(C)C)C(C)C)[C@H](n2cnc4c(=O)[nH]c(NC(=O)N(C)C)nc42)O3)(c2ccccc2)c2ccc(OC)cc2)cc1]

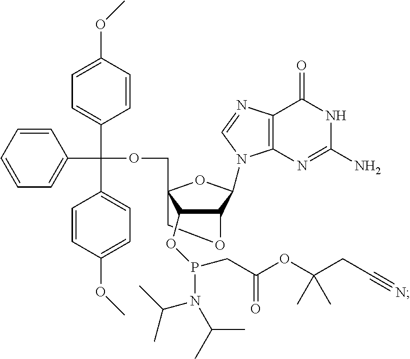 [SMILES: COc1ccc(C(OC[C@]23CO[C@@H](C2OP(CC(=O)OC(C)(C)CC#N)N(C(C)C)C(C)C)[C@H](n2cnc4c(=O)[nH]c(N)nc42)O3)(c2ccccc2)c2ccc(OC)cc2)cc1]

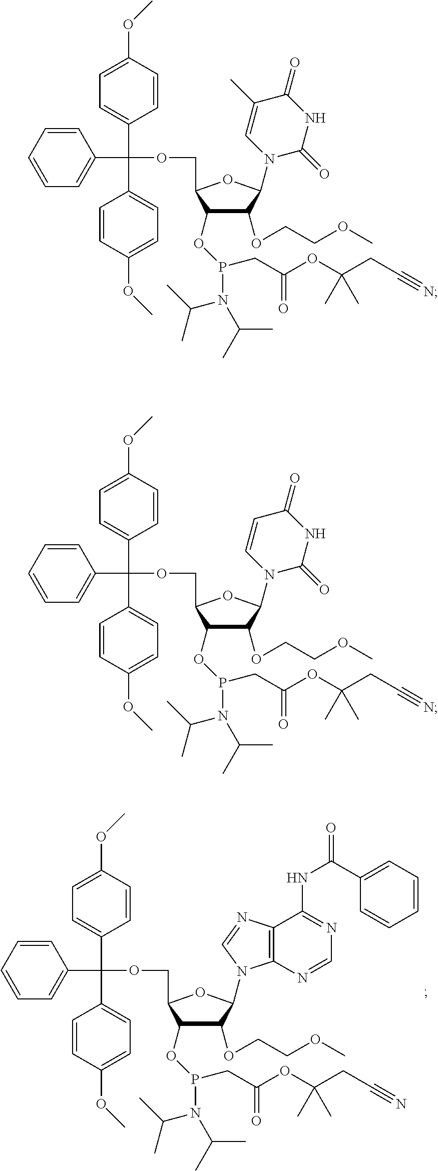

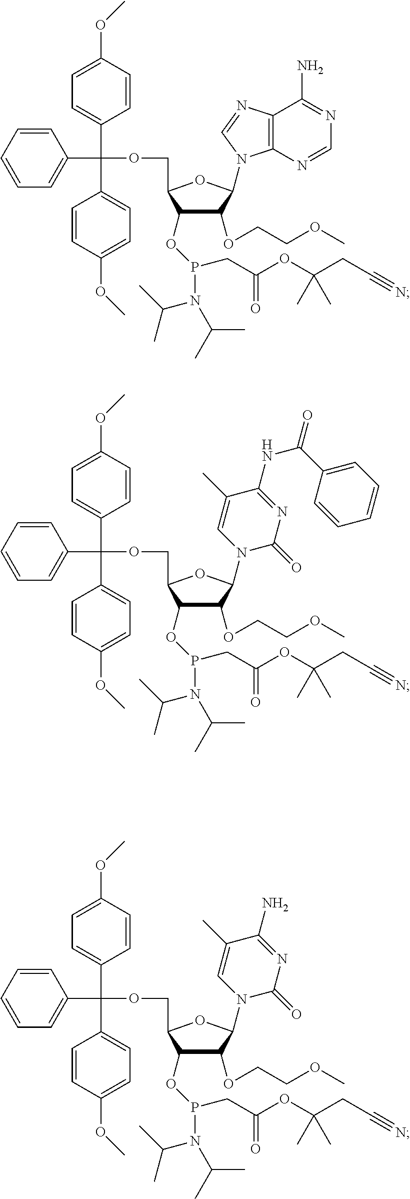

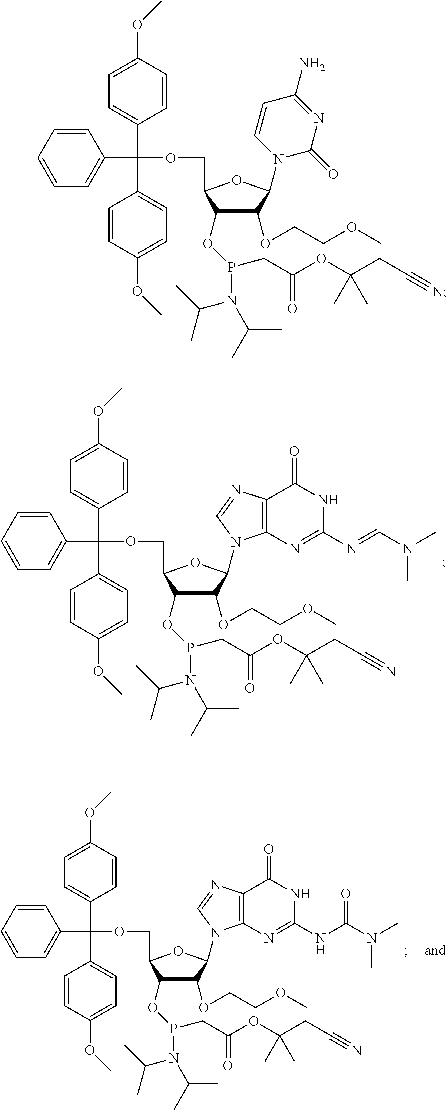

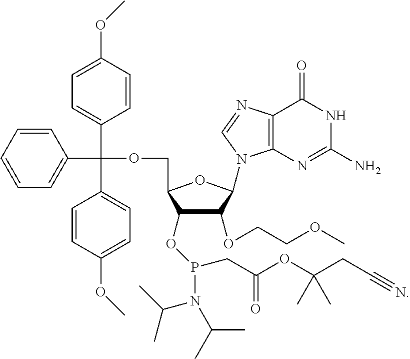

13. A process for the manufacture of a compound of formula (I-a) according to claim 1 comprising the reaction of a compound of formula (E) 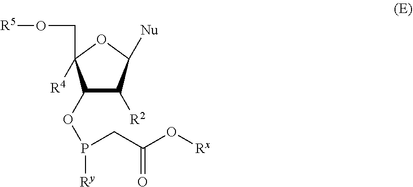
with a compound of formula P(R^{y})₂(CH₂)COO(R^{x}) in the presence of a coupling agent, wherein X, Y, R⁵, Nu, R^{x} and R^{y} are as defined in claim 1.

14. The process according to claim 13 comprising the reaction of a compound of formula (C) or (D) 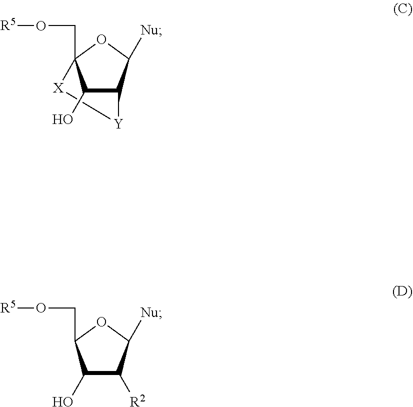
with a compound of formula P(R^{y})₂(CH₂)COO(R^{x}) in the presence of a coupling agent, wherein X, Y, R⁵, Nu, R^{x} and R^{y} are as defined in claim 1.

15. The process according to claim 13, wherein the coupling agent is 1H-tetrazole, 5-ethylthio-1H-tetrazole, 2-benzylthiotetrazole or 4,5-dicyanoimidazole (DCI).

16-17. (canceled)
